SEQUENCE LISTING (51 sequences)

SEQ ID NO: 1 - RNA, 21 nt, Artificial Sequence
  Description of Artificial Sequence Synthetic oligonucleotide
agaagugaugccacuugguuu

SEQ ID NO: 2 - RNA, 21 nt, Artificial Sequence
  Description of Artificial Sequence Synthetic oligonucleotide
accaaguggcaucacuucuuu

SEQ ID NO: 3 - RNA, 21 nt, Artificial Sequence
  Description of Artificial Sequence Synthetic oligonucleotide
guacaagugugagaagugcuu

SEQ ID NO: 4 - RNA, 21 nt, Artificial Sequence
  Description of Artificial Sequence Synthetic oligonucleotide
gcacuucucacacuuguacuu

SEQ ID NO: 5 - RNA, 21 nt, Artificial Sequence
  Description of Artificial Sequence Synthetic oligonucleotide
ggucuugaggaaagaccuguu

SEQ ID NO: 6 - RNA, 21 nt, Artificial Sequence
  Description of Artificial Sequence Synthetic oligonucleotide
caggucuuuccucaagaccuu

SEQ ID NO: 7 - RNA, 21 nt, Artificial Sequence
  Description of Artificial Sequence Synthetic oligonucleotide
uggagugcucaucuggaaguu

SEQ ID NO: 8 - RNA, 21 nt, Artificial Sequence
  Description of Artificial Sequence Synthetic oligonucleotide
cuuccagaugagcacuccauu

SEQ ID NO: 9 - RNA, 21 nt, Artificial Sequence
  Description of Artificial Sequence Synthetic oligonucleotide
cuccucugggggauuugaauu

SEQ ID NO: 10 - RNA, 21 nt, Artificial Sequence
  Description of Artificial Sequence Synthetic oligonucleotide
uucaaaucccccagaggaguu

SEQ ID NO: 11 - RNA, 21 nt, Artificial Sequence
  Description of Artificial Sequence Synthetic oligonucleotide
ucucaaagcucgcgggacauu

SEQ ID NO: 12 - RNA, 21 nt, Artificial Sequence
  Description of Artificial Sequence Synthetic oligonucleotide
ugucccgcgagcuuugagauu

SEQ ID NO: 13 - RNA, 21 nt, Artificial Sequence
  Description of Artificial Sequence Synthetic oligonucleotide
gggaaagcgucgcagcaaauu

SEQ ID NO: 14 - RNA, 21 nt, Artificial Sequence
  Description of Artificial Sequence Synthetic oligonucleotide
uuugcugcgacgcuuucccuu

SEQ ID NO: 15 - RNA, 21 nt, Artificial Sequence
  Description of Artificial Sequence Synthetic oligonucleotide
cgccaaaucaagccaauuauu

SEQ ID NO: 16 - RNA, 21 nt, Artificial Sequence
  Description of Artificial Sequence Synthetic oligonucleotide
uaauuggcuugauuuggcguu

SEQ ID NO: 17 - RNA, 21 nt, Artificial Sequence
  Description of Artificial Sequence Synthetic oligonucleotide
ucaccaagccucugaaggauu

SEQ ID NO: 18 - RNA, 21 nt, Artificial Sequence
  Description of Artificial Sequence Synthetic oligonucleotide
uccuucagaggcuuggugauu

SEQ ID NO: 19 - RNA, 21 nt, Artificial Sequence
  Description of Artificial Sequence Synthetic oligonucleotide
ccacaugugccgagaauucuu

SEQ ID NO: 20 - RNA, 21 nt, Artificial Sequence
  Description of Artificial Sequence Synthetic oligonucleotide
gaauucucggcacaugugguu

SEQ ID NO: 21 - RNA, 21 nt, Artificial Sequence
  Description of Artificial Sequence Synthetic oligonucleotide
ccaaggaaccaauuuaucauu

SEQ ID NO: 22 - RNA, 21 nt, Artificial Sequence
  Description of Artificial Sequence Synthetic oligonucleotide
ugauaaauugguuccuugguu

SEQ ID NO: 23 - RNA, 21 nt, Artificial Sequence
  Description of Artificial Sequence Synthetic oligonucleotide
caacaucuacaucuacaauuu

SEQ ID NO: 24 - RNA, 21 nt, Artificial Sequence
  Description of Artificial Sequence Synthetic oligonucleotide
auuguagauguagauguuguu

SEQ ID NO: 25 - RNA, 21 nt, Artificial Sequence
  Description of Artificial Sequence Synthetic oligonucleotide
gaaccaauuuaucacccauuu

SEQ ID NO: 26 - RNA, 21 nt, Artificial Sequence
  Description of Artificial Sequence Synthetic oligonucleotide
augggugauaaauugguucuu

SEQ ID NO: 27 - RNA, 21 nt, Artificial Sequence
  Description of Artificial Sequence Synthetic oligonucleotide
gauaagguguuuaaggauauu

SEQ ID NO: 28 - RNA, 21 nt, Artificial Sequence
  Description of Artificial Sequence Synthetic oligonucleotide
uauccuuaaacaccuuaucuu

SEQ ID NO: 29 - RNA, 21 nt, Artificial Sequence
  Description of Artificial Sequence Synthetic oligonucleotide
gcaaguggcucggaacacauu

SEQ ID NO: 30 - RNA, 21 nt, Artificial Sequence
  Description of Artificial Sequence Synthetic oligonucleotide
uguguuccgagccacuugcuu

SEQ ID NO: 31 - RNA, 21 nt, Artificial Sequence
  Description of Artificial Sequence Synthetic oligonucleotide
gacaucugcuggugcauuuuu

SEQ ID NO: 32 - RNA, 21 nt, Artificial Sequence
  Description of Artificial Sequence Synthetic oligonucleotide
aaaugcaccagcagaugucuu

SEQ ID NO: 33 - RNA, 21 nt, Artificial Sequence
  Description of Artificial Sequence Synthetic oligonucleotide
ggcccaaacuguucuagaauu

SEQ ID NO: 34 - RNA, 21 nt, Artificial Sequence
  Description of Artificial Sequence Synthetic oligonucleotide
uucuagaacaguuugggccuu

SEQ ID NO: 35 - RNA, 21 nt, Artificial Sequence
  Description of Artificial Sequence Synthetic oligonucleotide
caaagaagcugacaauacuuu

SEQ ID NO: 36 - RNA, 21 nt, Artificial Sequence
  Description of Artificial Sequence Synthetic oligonucleotide
aguauugucagcuucuuuguu

SEQ ID NO: 37 - RNA, 21 nt, Artificial Sequence
  Description of Artificial Sequence Synthetic oligonucleotide
ccagauacuuucuuuacuauu

SEQ ID NO: 38 - RNA, 21 nt, Artificial Sequence
  Description of Artificial Sequence Synthetic oligonucleotide
uaguaaagaaaguaucugguu

SEQ ID NO: 39 - RNA, 21 nt, Artificial Sequence
  Description of Artificial Sequence Synthetic oligonucleotide
gaaguucgguuuaguagccuu

SEQ ID NO: 40 - RNA, 21 nt, Artificial Sequence
  Description of Artificial Sequence Synthetic oligonucleotide
ggcuacuaaaccgaacuucuu

SEQ ID NO: 41 - RNA, 21 nt, Artificial Sequence
  Description of Artificial Sequence Synthetic oligonucleotide
aaauauggcaucuccuuaauu

SEQ ID NO: 42 - RNA, 21 nt, Artificial Sequence
  Description of Artificial Sequence Synthetic oligonucleotide
uuaaggagaugccauauuuuu

SEQ ID NO: 43 - RNA, 21 nt, Artificial Sequence
  Description of Artificial Sequence Synthetic oligonucleotide
ggagcauuggccucaaggauu

SEQ ID NO: 44 - RNA, 21 nt, Artificial Sequence
  Description of Artificial Sequence Synthetic oligonucleotide
uccuugaggccaaugcuccuu

SEQ ID NO: 45 - RNA, 21 nt, Artificial Sequence
  Description of Artificial Sequence Synthetic oligonucleotide
gcagguacggcuacaagauuu

SEQ ID NO: 46 - RNA, 21 nt, Artificial Sequence
  Description of Artificial Sequence Synthetic oligonucleotide
aucuuguagccguaccugcuu

SEQ ID NO: 47 - RNA, 21 nt, Artificial Sequence
  Description of Artificial Sequence Synthetic oligonucleotide
cgguagagggugagaaacauu

SEQ ID NO: 48 - RNA, 21 nt, Artificial Sequence
  Description of Artificial Sequence Synthetic oligonucleotide
uguuucucacccucuaccguu

SEQ ID NO: 49 - RNA, 21 nt, Artificial Sequence
  Description of Artificial Sequence Synthetic oligonucleotide
gaagaaggcauuucuauuuuu

SEQ ID NO: 50 - RNA, 21 nt, Artificial Sequence
  Description of Artificial Sequence Synthetic oligonucleotide
aaauagaaaugccuucuucuu

SEQ ID NO: 51 - RNA, 20 nt, Artificial Sequence
  Description of Artificial Sequence Synthetic oligonucleotide
uagcgaccuaaacacaucaa

(19) United States
(12) Patent Application Publication
(10) Pub. No.: US 2012/0282245 A1
(43) Pub. Date: Nov. 8, 2012

(54) BIOMARKERS AND ASSAYS FOR THE TREATMENT OF CANCER

(75) Inventors: Matvey E. Lukashev, Tewksbury, MA (US); Pradeep Bista, Arlington, MA (US); Weike Zeng, Winchester, MA (US)

(73) Assignee: BIOGEN IDEC MA INC., CAMBRIDGE, MA (US)

(21) Appl. No.: 12/443,936

(22) PCT Filed: Oct. 3, 2007
(86) PCT No.: PCT/US07/21356

§ 371 Date: Dec. 8, 2009

Related U.S. Application Data
(60) Provisional application No. 60/848,956, filed on Oct. 3, 2006.

(51) Int. Cl. G01N 33/574; G01N 33/561; G01N 27/62; G01N 30/02; G01N 27/26; A61P 35/00; C40B 40/06; A61K 39/395; A61K 31/713; A61K 31/7088; A61K 33/24; C12Q 1/68; G01N 30/90

(52) U.S. Cl. 424/130.1; 435/6.11; 435/7.92; 435/7.1; 506/9; 435/29; 506/16; 514/44 A; 424/649

(57) ABSTRACT

This invention features methods for prognosticating the efficacy of a cancer treatment comprising administration of a lymphotoxin-β receptor (LT-β-R) using TRAF3, TRAF2, and/or p53 markers, as well as combination therapies that include a composition that activates lymphotoxin-beta receptor signaling in combination with one or more other agents.

### RELATED APPLICATIONS

This patent application claims the benefit of U.S. Provisional Patent Application Ser. No. 60/848,956, entitled “Biomarkers and Assays for the Treatment of Cancer”, filed Oct. 3, 2006. The entire contents of the above-referenced provisional patent application are incorporated herein by this reference.

### BACKGROUND OF THE INVENTION

Lymphotoxin beta receptor (referred to herein as LT-β-R) is a member of the tumor necrosis factor family which has a well-described role both in the development of the immune system and in the functional maintenance of a number of cells in the immune system including follicular dendritic cells and a number of stromal cell types (Crowe el at (1994) Science 264:707; Browning et al. (1993) 72: 847; Browning et al. (1995) 154:33; Matsumoto et al. (1997) Immol. Rev. 156:137).

Lymphotoxin beta Receptor (LTβR) belongs to a subset of the Tumor Necrosis Factor Receptor (TNFR) superfamily that can activate both the canonical (p105/p50-driven, also termed NFκB1) and the alternative (p100/p52-driven, also termed NFκB2) pathways of Nuclear Factor of kappa-B (NFκB)-dependent gene transcription (Bonizzi, G. and M. Karin (2004). Trends Immunol 25(6): 280-8; Hayden, M. S, and S. Ghosh (2004). Genes Dev 18(18): 2195-224). This subset of receptors also includes CD40, BAFF-R, Fn14, and RANK, while other TNFRs are unable to activate the alternative arm (Bonizzi and Karin 2004; Hayden and Ghosh 2004). Upon receptor stimulation by ligand, NFκB1 activation is rapid, and involves the Inhibitor of kappa-B kinase (IKK)-complex mediated phosphorylation, and subsequent degradation of the inhibitor IκBα, to allow p50-mediated gene transcription (Beinke, S, and S. C. Ley (2004). Biochem J 382(Pt 2): 393-409). On the other hand, activation of the alternative arm of NFκB is delayed, and involves NFκB-inducing kinase (NIK) and IKKα-mediated processing of the p100 component of NFκB2, into the transcriptionally active p52 fragment (Beinke and Ley 2004).

Receptors that only activate NFκB1, such as the prototypic TNFR1, are involved in inflammatory and innate immune responses (Locksley, R. M., N. Killeen, et al (2001). Cell 104(4): 487-501, and are transducers of transient signals that are self-limiting via a negative-regulatory loop with NFκB1-dependent resynthesis of the inhibitor IκBα (Sun, S. C., P. A. Ganchi, et al. (1993). Science 259(5103): 1912-5). NFκB2 signal, by contrast, is sustained, and is important in developmental processes, for example, in peripheral lymphoid organogenesis (Franzoso, G., L. Carlson, et al. (1998). J Exp Med 187(2): 147-59; Senftleben, U., Y. Cao, et al (2001). Science 293(5534): 1495-9; Gommerman, J. L. and J. L. Browning (2003). Nat Rev Immunol 3(8): 642-55), and in osteoclastogenesis (Franzoso, G., L. Carlson, et al. (1997). Genes Dev 11(24): 3482-96). For receptors that activate both NFκB pathways, it therefore seems necessary to limit NFκB1 activation in order to prevent inflammatory responses and to initiate prolonged NFκB2 activation to complete regulated developmental processes. These receptors, including LTβR, therefore control unique patterns of gene expression that are differentially regulated by NFκB1- and NFκB2-pathways (Dejardin, Droin et al. 2002 Immunity 17(4): 525-35; Muller and Siebenlist 2003, J Biol Chem 278(14): 12006-12). And it has been shown previously that this subset of receptors facilitate this switch by sequentially exchanging NFκB dimers upon activation (Saccani, S., S. Pantano, et al (2003). Mol Cell 11(6): 1563-74). However, the mechanism(s) by which the receptors switch NFκB activities, or how NFκB2 activation is sustained for long periods, is not known.

LTβR signal transduction requires one or more of several TNFR-associated factors (TRAFs), which are adapter molecules that bridge the receptors to downstream kinases (Bradley, J. R. and J. S. Pober (2001) Oncogene 20(44): 6482-91; Chung, J. Y., Y. C. Park, et al. (2002). J Cell Sci 115(Pt 4): 679-88). Upon stimulation, LTβR recruits TRAF2 and TRAF3 in receptor associated signaling complexes (Force, W. R., A. A. Glass, et al., (2000). J Biol Chem 275(15): 11121-9; Kuai, J., E. Nickbarg, et al (2003). J Biol Chem 278(16): 14363-9). TRAF2 has been implicated as a component in the transduction of NFκB signals from LTβR (Kuai, Nickbarg et al. 2003; Kim, Y. S., S. A. Nedospasov, et al. (2005). Mol Cell Biol 25(6): 2130-7), while studies on TRAF3's role in LTβR signaling have been concentrated in its requirement to activated JNK and mediate cell death (F Force, W. R., T. C. Cheung, et al. (1997). J Biol Chem 272(49): 30835-40; VanArsdale, T. L., S. L. VanArsdale, et al. (1997). Proc Natl Acad Sci USA 94(6): 2460-5). Moreover, activating and inhibitory roles, respectively, for TRAF2 and 3, in CD40 and BAFF—R signaling, have also been reported (Hostager, B. S. and G. A. Bishop (1999). J Immunol 162(11): 6307-11; Xu, L. G. and H. B. Shu (2002). J Immunol 169(12): 6883-9). A recent study using fluorescence energy transfer between ectopically expressed TRAF2 and TRAF3 suggests that these molecules are recruited in close proximity to one another at the CD40 receptor during signaling, and that increasing the levels of TRAF3 in the receptor complex inhibits TRAF2-mediated NFκB activation (He, L., A. C. Grammer, et al. (2004). J Biol Chem 279(53): 55855-65). This is supported by another study showing TRAF3's role as an inhibitor of CD40 signals (Hostager and Bishop 1999). A further study shows, in an overexpression system, that TRAF3 also inhibits NFκB2 activation (Hauer, J., S. Puschner, et al. (2005). Proc Natl Acad Sci USA 102(8): 2874-9), and is consistent with the report that TRAF3 represses MK (Liao, G., M. Zhang, et al. (2004). J Biol Chem 279(25): 26243-50), a kinase that is required for NFκB2 activation (Xiao, G., E. W. Harhaj, et al. (2001). Mol Cell 7(2): 401-9).

Cancer is one of the most prevalent health problems in the world today, affecting approximately one in five individuals in the United States. Many molecules have been identified on tumor cells as potential targets for antibody based therapy. Activation of LT-β-R has been shown to induce the apoptotic death of certain cancer cell lines in vivo (PCT/US96/01386). Prognostic markers that will identify patients who are likely (or those unlikely) to respond to treatment with LT-β-R activating agents will aid and improve clinical treatment decisions. Furthermore, methods of enhancing the anti-tumor effects of LT-β-R activating agents will also be useful for treating or reducing the advancement, severity or effects of cancer in subjects (e.g., humans).

### SUMMARY OF THE INVENTION

The present invention provides, in part, methods and kits for prognosticating the efficacy of a cancer treatment as well as methods for the treatment of cancer. More specifically, as described herein, TRAF3 has been identified as a key factor Controlling the coupling of LTβR to the canonical NFκB pathway, the latter of which is implicated as a mediator of the cytostatickytotoxic effect of LTβR activating agents, including agonist LTR antibodies, on tumor cells. As shown in the appended examples, certain tumors treated with a lymphotoxin-P receptor (LT-β-R) activating agent are resistant to the apoptotic effects of such a treatment, while other tumors are sensitive to such treatment. The Examples show that these resistant tumors have elevated levels of TRAF3 and that TRAF3 inhibits LT-β-R-induced NFκB1 signaling by limiting recruitment of TRAF2 and IKKα to the receptor and subsequent apoptosis of cancer cells. Furthermore, the Examples show that TRAF3 also independently inhibits LT-β-R-induced NFκB2 signaling. The present invention also demonstrates that a high percentage of tumors that are sensitive to treatment with a lymphotoxin-β receptor (LT-β-R) activating agent express p53 and those tumors that do not are resistant to treatment with LTBR. It has also been discovered that the ratio of the amount of TRAF3 to the amount of TRAF2 in a tumor is predictive of response to treatment with a lymphotoxin-β receptor (LT-β-R) activating agent.

The invention also provides a method for determining whether LT-β-R is active based on the ratio of TRAF3/TRAF2. Thus, the invention provides a method for determining LT-β-R signaling (presence or absence) using the ratio of TRAF3/TRAF2 (or vice versa), or, alternatively, the amount of TRAF2 and TRAF3 relative to a control marker.

Accordingly, the invention provides a method for predicting the sensitivity or resistance of a tumor to treatment with an LT-β-R activating agent comprising comparing the ratio of an amount of TRAF3 to an amount of TRAF2 present in the tumor with a known standard ratio of an amount of TRAF3 to an amount of TRAF2 present in a tumor with known sensitivity to treatment with an LT-β-R activating agent and/or a known standard ratio of an amount of TRAF3 to an amount of TRAF2 present in a tumor with known resistance to treatment with an LT-β-R activating agent, evaluating the TRAF3/TRAF2 ratio present in the tumor relative to the known standard ratio(s), thereby predicting the sensitivity or resistance of the tumor to treatment with an LT-β-R activating agent. In one embodiment, the tumor is predicted to be sensitive to the LT-β-R activating agent if the TRAF3/TRAF2 ratio obtained from the sample of the tumor prior to administration of the treatment is about equal to or less than the TRAF3/TRAF2 ratio obtained from a tumor with known sensitivity to treatment with the LT-β-R activating agent. In another embodiment, the tumor is predicted to be sensitive to the LT-β-R activating agent if the TRAF3/TRAF2 ratio obtained from the sample of the tumor prior to administration of the treatment is less than the TRAF3/TRAF2 ratio obtained from a tumor with known resistance to treatment with the LT-β-R activating agent. In another embodiment, the tumor is predicted to be resistant to the LT-β-R activating agent if the TRAF3/TRAF2 ratio obtained from the sample of the tumor prior to administration of the treatment is greater than the TRAF3/TRAF2 ratio obtained from a tumor with known sensitivity to treatment with the LT-β-R activating agent. In yet another embodiment, the tumor is predicted to be resistant to the LT-β-R activating agent if the TRAF3/TRAF2 ratio obtained from the sample of the tumor prior to administration of the treatment is about equal to or greater than the TRAF3/TRAF2 ratio obtained from a tumor with known resistance to treatment with the LT-β-R activating agent. In one embodiment, the invention provides a method for predicting the sensitivity or resistance of a tumor to treatment with an LT-β-R activating agent comprising comparing the ratio of an amount of TRAF3 to an amount of TRAF2 present in the tumor prior to treatment with a known standard ratio of an amount of TRAF3 to an amount of TRAF2 present in a tumor with known sensitivity to treatment with an LT-β-R activating agent and a known standard ratio of an amount of TRAF3 to an amount of TRAF2 present in a tumor with known resistance to treatment with an LT-β-R activating agent, evaluating the TRAF3/TRAF2 ratio present in the tumor relative to the known standard ratios, thereby predicting the sensitivity or resistance of the tumor to treatment with an LT-β-R activating agent.

The invention provides a method for predicting the sensitivity or resistance of a tumor to treatment with an LT-β-R activating agent comprising comparing the amount of TRAF3, e.g., by determining the level of expression of TRAF3, e.g., the nucleic acid or protein level, obtained from a sample of the tumor prior to administration of the treatment with the amount of TRAF3 obtained from a tumor with known sensitivity to treatment with an LT-β-R activating agent and a tumor with known resistance to treatment with an LT-β-R activating agent, evaluating the amount of TRAF3 in the sample relative to the known standard amount, thereby predicting the sensitivity or resistance of the tumor to treatment with an LT-β-R activating agent. In one embodiment, the tumor is predicted to be sensitive to the LT-β-R activating agent if the amount of TRAF3 obtained from the sample of the tumor prior to administration of the treatment is about equal to or less than the amount of TRAF3 obtained from a tumor with known sensitivity to treatment with the LT-β-R activating agent. In another embodiment, the tumor is predicted to be sensitive to the LT-β-R activating agent if the amount of TRAF3 obtained from the sample of the tumor prior to administration of the treatment is less than the amount of TRAF3 obtained from a tumor with known resistance to treatment with the LT-β-R activating agent. In another embodiment, the tumor is predicted to be resistant to the LT-β-R activating agent if the amount of TRAF3 obtained from the sample of the tumor prior to administration of the treatment is greater than the amount of TRAF3 obtained from a tumor with known sensitivity to treatment with the LT-β-R activating agent. In yet another embodiment, the tumor is predicted to be resistant to the LT-β-R activating agent if the amount of TRAF3 obtained from the sample of the tumor prior to administration of the treatment is about equal to or greater than the amount of TRAF3 obtained from a tumor with known resistance to treatment with the LT-β-R activating agent.

In another aspect, the invention provides a method for predicting the efficacy of a treatment for cancer comprising administration of a lymphotoxin-ft receptor (LT-β-R) activating agent to a subject having a tumor, said method comprising determining a TRAF3/TRAF2 ratio present in the tumor and comparing the TRAF3/TRAF2 ratio present in the tumor with a known standard TRAF3/TRAF2 ratio present in a tumor with known sensitivity to treatment with the LT-β-R activating agent, wherein a TRAF3/TRAF2 ratio present in the tumor which is approximately equal to or less than the known ratio predicts that the treatment will be efficacious for the treatment of cancer and a TRAF3/TRAF2 ratio present in the tumor which is greater than the known standard ratio predicts that the treatment will not be efficacious for the treatment of cancer.

In another aspect, the invention provides a method for predicting the efficacy of a treatment comprising administration of a lymphotoxin-3 receptor (LT-β-R) activating agent to a subject having cancer, said method comprising, obtaining a tumor tissue sample from the subject having cancer, wherein the sample is obtained prior to administration of the treatment, measuring the amount of TRAF3, e.g., by determining the level of expression of TRAF3, e.g., the nucleic acid or protein level, in the tissue sample, and comparing the foregoing amount with the normal amount of TRAF3, wherein an amount of TRAF3 in the tissue sample approximately equal to or less than the normal amount predicts that the treatment will be efficacious for the treatment of cancer and an amount of TRAF3 in the tissue sample greater than the normal amount predicts that the treatment will not be efficacious for the treatment of cancer.

In another aspect the invention provides a method for predicting the efficacy of a treatment comprising administration of a lymphotoxin-P receptor (LT-β-R) activating agent to a subject having cancer, said method comprising, obtaining a tumor tissue sample from the subject having cancer, wherein the sample is obtained prior to administration of the treatment, measuring the amount of TRAF2, e.g., by determining the level of expression of TRAF2, e.g., the nucleic acid or protein level, in the tissue sample, and comparing the foregoing amount with the normal amount of TRAF2, wherein an amount of TRAF2 in the tissue sample approximately equal to or less than the normal amount predicts that the treatment will be efficacious for the treatment of cancer and an amount of TRAF2 in the tissue sample greater than the normal amount predicts that the treatment will not be efficacious for the treatment of cancer.

Another aspect of the invention provides a method for predicting whether treatment of a tumor with an LT-β-R activating agent will be efficacious, comprising determining the amount of p53 in a sample of the tumor, wherein a significantly higher amount of p53 in the tissue sample relative to the normal amount of p53 predicts that the treatment of the tumor with an LT-β-R activating agent will be efficacious.

In yet another aspect, the invention provides a method for determining whether a subject having a tumor is a candidate for treatment with an LT-β-R activating agent, the method comprising, determining the amount of p53 present in a sample of the tumor, and comparing the amount of p53 present in a sample to a normal amount of p53, thereby determining whether the subject having the tumor is a candidate for treatment with an LT-β-R activating agent.

Yet another aspect of the invention provides a method for predicting the efficacy of a treatment comprising administration of a lymphotoxin-P receptor (LT-β-R) activating agent to a subject having cancer. The method comprises obtaining a tumor tissue sample from a patient having cancer, wherein the sample is obtained prior to the treatment, measuring the amount of p53 in the tissue sample, and comparing the amount of p53 in the tissue sample with the normal amount of p53, thereby predicting the efficacy of the treatment.

In another aspect, the invention provides a method for treating a cancerous tumor comprising administering to a subject having the cancerous tumor an LT-β-R activating agent and an agent that inhibits TRAF3 activity.

In yet another aspect, the invention provides a method for treating a cancerous tumor comprising administering to a subject having the cancerous tumor an LT-β-R activating agent and an NFκB1 activating agent.

In one embodiment, the treatment methods of the invention are administered in combination with a chemotherapeutic agent. In one embodiment, the chemotherapeutic agent is selected from the group consisting of gemcitabine, adriamycin, Camptosar, carboplatin, cisplatin, and Taxol.

In yet another aspect, the invention provides a method for increasing the efficacy of treatment of a tumor with a lymphotoxin-β receptor (LT-β-R) activating agent, comprising administering to a subject having said tumor, an agent which inhibits TRAF3 activity, such that the efficacy of treatment of the tumor with the LT-β-R activating agent is increased.

In one embodiment, the agent that inhibits TRAF3 activity is selected from the group consisting of an antibody, an siRNA molecule, and an antisense nucleic acid molecule.

In one embodiment of the methods of the invention, the LT-β-R activating agent comprises an anti-LT-β-R binding molecule. In one embodiment, the LT-β-R binding molecule comprises an anti-LT-β-R antibody, or an antigen-binding fragment thereof. In one embodiment, the anti-LT-β-R antibody is a humanized antibody, or antigen-binding fragment thereof. In one embodiment, the humanized antibody, or antigen-binding fragment thereof, comprises a variable region comprising complementary determining regions (CDRs) corresponding to CDRs from the mouse CBE11 antibody. In one embodiment, the humanized antibody, or antigen-binding fragment thereof, is humanized CBE11. In another embodiment, the anti-LT-β-R antibody, or antigen-binding fragment thereof; is a multivalent anti-LT-β-R antibody. In one embodiment, the multivalent anti-LT-β-R antibody comprises at least one CDR derived from the CBE11 antibody. In one embodiment, the anti-LT-β-R antibody is conjugated to a chemotherapeutic agent or an immunotoxin. In one embodiment, the chemotherapeutic agent is selected from the group consisting of gemcitabine, adriamycin, Camptosar, carboplatin, cisplatin, and Taxol.

In one embodiment of the methods of the invention, the tumor is a carcinoma. In one embodiment, the tumor is a colon tumor or a cervical tumor.

Another aspect of the invention provides a kit for performing the methods of the invention. The kit may comprise a detectable agent that specifically recognizes TRAF3, TRAF2, or p53, instructions for use, and, optionally, reagents for isolating a sample from a tumor cell.

### BRIEF DESCRIPTION OF THE FIGURES

FIG. 1 shows LTβR-specific activation of canonical NFκB1 is uncoupled in certain cells and correlates with differential cytokine gene activation. DID-1 and WiDr cells were treated with agonist LTβR antibodies (BS-1) at 100 ng/ml, or TNFα, (20 ng/ml) for the indicated times. Cells lysates were analyzed by Western blot for phosphorylated IκBα (A), phosphorylated RelA (B), and NFκB2 (D). RNA isolated from untreated cells (solid bars), or cells treated with anti-LTβR antibodies (hatched bars) for 4 hours were analyzed by real-time qPCR for IP-10 transcripts and the data reported as GAPDH normalized values from quadruplicates, shown as average+S.D. (C).

FIG. 2 shows late IκBα phosphorylation during LTβR activation is not impaired, and correlates with the LTβR-specific downregulation of TRAF3. (A) Cells were treated as in FIG. 1 for an extended time-course, and lysates were analyzed by Western blot for IκBα. Lysates from DLD-1 cells were also analyzed by Western blot for TRAF3. (B) Densitometry analysis from (A), of IκBα and TRAF3 protein expression from DLD-1 cells. (C) DLD-1 cells were left untreated, or treated for 10 minutes and 20 hours with 100 ng/ml anti-LTβR antibody (BS-1), or 20 ng/ml TNFα, and lysates were analyzed by Western blot for TRAF3.

FIG. 3 shows differential cellular TRAF3 protein expression leads to its differential recruitment at LTβR-signaling complexes. DLD-1 and WiDr cell lysates were Western blotted for TRAF2 and TRAF3, and protein expression levels were analyzed by densitometry (A). Cells were mock treated (− lanes), treated for 10 minutes (10′ lanes) and, 24 hours (24 h lanes) with 100 ng/ml anti-LTβR antibody, or treated with the anti-LTβR antibody after lysis as positive controls (* lanes). Lysates were immunoprecipitated using a secondary antibody against the anti-LTβR antibody, washed, and were Western blotted for LTβR, TRAF3, TRAF2 and IKKα, as indicated (B). The blots were analyzed by densitometry, and the results are shown as ratios of either TRAF2 (hatched bars), or TRAF3 (solid bars), to LTβR (C).

FIG. 4 shows that siRNA-mediated knockdown of TRAF3 restores LTβR-induced canonical NFκB activation. DLD-1 cells were mock transfected (Mock), or transfected with either a non-specific control siRNA (NS), or TRAF3 siRNA (TRAF3) for 48 hours. Cells were then left untreated (− lanes), or treated (+ lanes) with anti-LTβR antibody (BS-1) for 10 minutes. Lysates were Western blotted as shown (A). RNA was collected from these samples, treated for 4 hours with BS-1, and analyzed by real-time qPCR for IP-10 transcripts (B). Solid bars, untreated; hatched bars, anti-LTβA (BS-1) treated. Results are normalized to GAPDH transcripts and shown as an average+S.D. from quadruplicate samples.

FIG. 5 shows that TRAF3 knockdown restores canonical NFκB signaling, in part, by changing the recruitment of TRAF2 and IKKα to LTβR complexes. DLD-1 cells were transfected, and treated as in FIG. 4, and pre-IP lysates Western blotted for TRAF3 (A, top). Lysates were also immunoprecipitated using agarose-conjugated secondary antibody against the agonist anti-LTβR antibody (BS-1), washed, and Western blotted, as indicated, for TRAF2, TRAF3 and Unlit (A, bottom), and for IKKα (B). Blots from (A, bottom) were analyzed by densitometry for ratios of TRAF2, or TRAF3, to LTβR (C).

FIG. 6 shows that TRAF3 knockdown leads to the signal-independent (constitutive) MK-dependent activation of non-canonical NFκB2. DLD-1 cells transfected with a non-silencing control siRNA or TRAF3 siRNA for 48 hours were left untreated (− lanes) or treated (+ lanes) with 100 ng/ml anti-LTβR antibody (BS-1) for 10 minutes and 24 hours. Lysates were Western blotted for NFκB2 (p100, and processed p52 are indicated by arrows) (A). Cells were mock transfected, transfected with siRNA as in (A), or co-transfected with additional siRNA directed against NIX, alone (NIK) or in combination (TRAF3+NIK), then left untreated or treated with BS-1 for 24 hours. Lysates were blotted for NFκB2 and NIK (B). NFκB2 p52 bands from (B) were analyzed by densitometry in (C). (D) DLD-1 cells were transfected with the indicated siRNA for 48 h in the absence of any stimulus and lysates were Western blotted for the indicated proteins.

FIG. 7 shows NFκB2 positive autoregulation is suppressed by TRAF3. (A) DLD-1 cells mock transfected (Mock), or transfected with a non-silencing siRNA (NS) or TRAF3 siRNA (TRAF3). Forty-eight hours post-transfection, cells were left untreated (None, solid bars), or were treated with agonist anti-LTβR antibody (BS-1, hatched bars) for 4 hours. RNA was collected and real-time qPCR was performed to measure RelB and p100 transcripts. Results are normalized to GAPDH transcripts, and represent averages of quadruplicate samples+S.D. (B) DLD-1 cells were transfected with TRAF3 alone, or in combination with components of NFκB without any stimulation. RNA was prepared 48 hours after transfection for the real-time qPCR quantitation of RelB and p100 RNA. (C) WiDr cells were transfected with a non-silencing siRNA (NS) or siRNA against the indicated targets for 48 hours, and subsequently treated for 24 hours with agonist anti-LTβR antibody (BS-1). Lysates were blotted as indicated.

FIG. 8 shows results from xenograft experiments using CBE11 to treat colorectal xenografts.

### DETAILED DESCRIPTION OF THE INVENTION

### 1. Definitions

For convenience, before further description of the present invention, certain terms employed in the specification, examples and appended claims are defined here.

The singular forms “a”, “an”, and “the” include plural references unless the context clearly dictates otherwise.

The term “administering” includes any method of delivery of a pharmaceutical composition or therapeutic agent into a subject's system or to a particular region in or on a subject. The phrases “systemic administration,” “administered systemically”, “peripheral administration”, and “administered peripherally” as used herein mean the administration of a compound, drug or other material other than directly into the central nervous system, such that it enters the subject's system and, thus, is subject to metabolism and other like processes, for example, subcutaneous administration. “Parenteral administration” and “administered parenterally” means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intra-articular, subcapsular, subarachnoid, intraspinal and infrasternal injection and infusion.

The term “TRAF” refers to INF Receptor Associated Factor (See e.g., Wajant et al, 1999, Cytokine Growth Factor Rev 10:15-26). Six members of the TRAF family of proteins have been identified in mammalian cells (IRAF1″, “TRAF2”, “TRAF3”, “TRAF4”, “TRAF5”, and “TRAF6”) (reviewed in Arch, R H., et al. 1998. Genes Dev. 12, 2821-2830 and Lee and Lee (2002) J. Biochem. 35:61, the contents of each of which are incorporated by reference). Members of the “TRAF” family are cytoplasmic adapter proteins which participate in the tumor necrosis factor receptor (“TNFR”) superfamily signal transduction cascade (see e.g., Arch, R. H. et al, 1998, Genes Dev. 12:2821-2830). TRAF proteins associate with the cytoplasmic domains of members of the tumor TNFR superfamily and modulate the signaling pathways in response to receptor engagement. All TRAF proteins, with the exception of TRAF1, contain an amino terminal RING finger domain with a characteristic pattern of cysteines and histidines that coordinate the binding of Zn2+ ions (Borden, K. L. B., et al. 1995. EMBO J 14, 1532-1521), followed by a stretch of multiple zinc fingers. All TRAFs also share a highly conserved carboxy-terminal domain (TRAF-C domain) which is required for receptor binding and can be divided into two parts, a highly conserved domain which mediates homo- and hetero-dimerization of TRAF proteins as well as the association of the adapter proteins with their associated receptors, and an amino-terminal half that displays a coiled-coil configuration. TRAF molecules have distinct patterns of tissue distribution, are recruited by different cell surface receptors and have distinct functions as revealed most clearly by the analysis of TRAF-deficient mice (see Lomaga, M. A., et al. 1999. Genes Dev. 13, 1015-24; Nakano, H., et al. 1999. Proc. Natl. Acad. Sci. USA 96, 9803-9808; Nguyen, L. T., et al. 1999. Immunity 11, 379-389; Xu, Y., et al. 1996. Immunity 5, 407-415; Yeh, W. C., et al 1997. Immunity 7, 715-725).

The nucleotide and amino acid sequence of “TRAF2” is known and can be found in, for example, GenBank accession No.: gi:42544228, the contents of which are incorporated herein by reference.

The nucleotide and amino acid sequence of the three isoforms of “TRAF3” are known and can be found in, for example, GenBank accession Nos.: gi:22027617, gi:22027619, and gi:22027615, the contents of which are incorporated herein by reference.

The term “agent that inhibits TRAF3 activity” refers to compounds that inhibit a biological activity of TRAF3. In one embodiment, a TRAF3 activity is apoptosis. Exemplary TRAF3 inhibitors include, but are not limited to e.g., binding molecules, nucleic acid molecules, e.g., antisense nucleic acid molecules, siRNA molecules, polypeptides or proteins.

The term “NFκB” or “NF-kappa-B” refers to a pleiotropic family of transcription factors that consists of homodimeric and heterodimeric complexes formed from combinations of members of the Rel family of proteins. The Rel family of proteins are related to the viral rel oncogene found in the Reticuloendotheliosis Virus Strain T, a replication-defective, acutely transforming type. NFκB is expressed in numerous different cell types after stimulation and/or cell activation by a wide variety of stimuli (e.g., cytokines, growth factors and mitogens, hormones, receptor ligation, crosslinking of surface molecules, viruses and viral proteins, oxidative stress, and chemical agents such as phorbol esters). NFκB-responsive genes include, for example, those encoding a number of cytokines and growth factors, cytokine receptors, receptor signaling proteins, cell adhesion molecules, and many other proteins involved in various processes, including immune responses, acute phase reaction and inflammation, cell growth and differentiation, growth control of certain tumors, and cell death by apoptosis.

There are five mammalian members of the Rel family of proteins. These include “c-Rel” (Brownell, et al. (1986) Am. J. Hum. Genet. 39: 194-202), “NFκB1” (also referred to as “NF-kappa-B1”, “p105”, or “p50”) (Meyer et al (1991 Proc Natl Acad Sci (USA) 88: 966-970), “NFκB2” (also referred to as “NF-kappa-B2”, “Lyt10” “p100”, or “p52”), “RelA” (also referred to as “p65” or “NF-kappa-B subunit 3”) (Deloukas et al (1993) Human Molecular Genetics 2: 1895-1900), and “RelB”. The most common form of NFκB found in virtually all cell types is composed of two subunits of 50 kDa (p50; derived from p105, a 105 kDa precursor) and 65 kDa (p65). The p50 and p52 subunits primarily serve as DNA binding subunits. RelA (p65), RelB, and c-Rel are responsible for gene activation in vivo. Homodimers of p50 cause transcriptional repression. Specific regulation of gene expression by NFκB is due to the differing cell type specificity of the heterodimeric forms of NFκB, DNA binding site preference, interaction with inhibitory proteins, activation requirements, and kinetics of activation (Miyamoto and Verma (1995) Proc Natl Acad Sci (USA) 91: 5056-5060).

NFκB dimers are regulated at the level of synthesis as some of the subunit genes contain NFκB binding sites in their promoter regions. Activities of NFκB are also regulated by inhibitory proteins. Inactive NFκB preexists in the cytoplasm of cells where complexed by the inhibitor “IκB” (also referred to as “I-kappa-B” (Inhibitor of NF-kappa-B). Mammalian IκB constitutes a protein family that includes IκBα (also referred to as “I-kappa-B-alpha”), IκBδ (also referred to as “I-kappa-B-beta”), IκBγ (also referred to as “I-kappa-B-gamma”), 1×138 (also referred to as “I-kappa-B-delta”), IκBε (also referred to as “I-kappa-B-epsilon”) and BCL3 (Verma, et al (1997) Proc Natl Acad Sci (USA) 94: 11758-11760; Miyamoto and Verma (1995) Advances in Cancer Research 66: 255-292; Siebenlist, et al (1994) Annual Review of Cell Biology 10: 405-455; Beg and Baldwin (1993) Genes and Development 7: 2064-2070; Kerr, et al (1992) Genes and Development 6, 2352-2363). Cytoplasmic sequestering of NFκB results from masking of the nuclear localization sequence of NFκB proteins (Beg et al (1992) Genes and Development 6: 1899-1913). These inhibitory proteins display differential affinities for different NFκB dimers. NFκB dimers can induce their own inhibitors, which then bind to cytoplasmic dimers to restore the inhibited state and reestablish cytoplasmic pools of NFκB/IκB complexes. Various activating agents can mediate the dissociation of IκB from NFκB and, thus, translocation of the liberated transcription factor into the nucleus. This process involves the activities of other kinases such as IKK-1 (1-kappa-B kinase-1; also IKK-alpha), IKK-2 (1-kappa-B kinase-2; also IKK-beta), and NEMO (LICK-gamma), which themselves are subject to phosphorylation and concomitant activation by kinases such as the NFκB inducible kinase NIK (Verma et al (1995) Genes and Development 9: 2723-2735). Signal dependent phosphorylation results in the ubiquitination of 1-kappa-B proteins and targets the cytoplasmic inhibitors to the ubiquitin-proteasome pathway (Chen et al (1995) Genes and Development 9: 1586-1597; Li et al (1995) Science 284: 321-325; Alkalay et al (1995) Proc Natl Acad Sci (USA) 92: 10599-10603).

The nucleotide and amino acid sequence of “NFκB1” is known and can be found in, for example, GenBank accession No.: gi:34577121, the contents of which are incorporated herein by reference.

The nucleotide and amino acid sequence of “NFκB2” is known and can be found in, for example, GenBank accession No.: gi:19923222, the contents of which are incorporated herein by reference.

The term “NFκB1 activating agent” refers to an agent that activates the NFκB1 pathway by activation of the transcription factor NFκB, and which is at least partially mediated by the NFκB factor (Karin, 1998, Cancer J from Scientific American, 4:92-99; Wallach et al, 1999, Ann Rev of Immunology, 17:331-367) inducing, for example, apoptotic cell death. Exemplary NFκB1 activating agents include, but are not limited to e.g., binding molecules (agonistic binding molecules), nucleic acid molecules, and polypeptides or proteins.

As used herein the term “apoptosis” includes programmed cell death which can be characterized using techniques which are known in the art. Apoptotic cell death can be characterized, e.g., by cell shrinkage, membrane blebbing and chromatin condensation culminating in cell fragmentation. Cells undergoing apoptosis also display a characteristic pattern of internucleosomal DNA cleavage.

As used herein, “p53” refers to the tumor suppressor protein p53 (also referred to as “TP53”) involved in the regulation of cell proliferation, which is well known in the art. The nucleotide and amino acid sequence of human p53 are known and can be found in, for example, GenBank Accession Nos.: gi:8400737 and gi:8400738, the contents of which are incorporated herein by reference.

The term “lymphotoxin β receptor” (“LT-β-R”) refers to the art known member of the tumor necrosis factor (TNF) receptor superfamily of molecules which mediates a wide range of innate and adaptive immune response functions (for a review, see, e.g., Gommerman and Browning (2003) Nat Rev 3:642, the contents of which are incorporated by reference).

The term “lymphotoxin β receptor” (LT-β-R) activating agent” refers to an agent that activates the LT-β-R pathway by signaling through the LT-β-R receptor inducing, for example, apoptotic cell death. Exemplary LT-β-R activating agents include, but are not limited to e.g., binding molecules (agonistic binding molecules), nucleic acid molecules, and polypeptides or proteins.

The term “binding molecule” refers to a molecule that comprises at least one binding domain which comprises a binding site that specifically binds to a target molecule (such as an antigen). For example, in one embodiment, a binding molecule for use in the methods of the invention comprises an immunoglobulin antigen binding site or the portion of a ligand molecule that is responsible for receptor binding.

In one embodiment, the binding molecule comprises at least two binding sites. In one embodiment, the binding molecules comprise two binding sites. In one embodiment, the binding molecules comprise three binding sites. In another embodiment, the binding molecules comprise four binding sites.

The term “LT-β-R binding molecule” refers to a molecule that comprises at least one lymphotoxin beta receptor (LT-β-R) binding site. Examples of LT-β-R binding molecules, including LT-β-R antibodies, which can be used in the methods and articles of manufacture of the invention include, but are not limited to, binding molecules described in WO 96/22788, WO 02/30986, and WO 04/002431, each of which is incorporated in its entirety by reference herein.

In one embodiment, the binding molecules of the invention are “antibody” or “immunoglobulin” molecules, e.g., naturally occurring antibody or immunoglobulin molecules or genetically engineered antibody molecules that bind antigen in a manner similar to antibody molecules. As used herein, the term “immunoglobulin” includes a polypeptide having a combination of two heavy and two light chains whether or not it possesses any relevant specific immunoreactivity. “Antibodies” refers to such assemblies which have significant known specific immunoreactive activity to an antigen. Antibodies and immunoglobulins comprise light and heavy chains, with or without an interchain covalent linkage between them. Basic immunoglobulin structures in vertebrate systems are relatively well understood.

The generic term “immunoglobulin” comprises five distinct classes of antibody that can be distinguished biochemically. All five classes of antibodies are clearly within the scope of the present invention, the following discussion will generally be directed to the IgG class of immunoglobulin molecules. With regard to IgG; immunoglobulins comprise two identical light polypeptide chains of molecular weight approximately 23,000 Dahons, and two identical heavy chains of molecular weight 53,000-70,000. The four chains are joined by disulfide bonds in a “Y” configuration wherein the light chains bracket the heavy chains starting at the mouth of the “Y” and continuing through the variable region.

Both the light and heavy chains are divided into regions of structural and functional homology. The terms “constant” and “variable” are used functionally. In this regard, it will be appreciated that the variable domains of both the light (VL) and heavy (VH) chain portions determine antigen recognition and specificity. Conversely, the constant domains of the light chain (CL) and the heavy chain (CH1, CH2 or CH3) confer important biological properties such as secretion, transplacental mobility, Fc receptor binding, complement binding, and the like. By convention the numbering of the constant region domains increases as they become more diital from the antigen binding site or amino-terminus of the antibody. The N-terminus is a variable region and at the C-terminus is a constant region; the CH3 and CL domains actually comprise the carboxy-terminus of the heavy and light chain, respectively.

Light chains are classified as either kappa or lambda (κ, λ). Each heavy chain class may be bound with either a kappa or lambda light chain. In general, the light and heavy chains are covalently bonded to each other, and the “tail” portions of the two heavy chains are bonded to each other by covalent disulfide linkages or non-covalent linkages when the immunoglobulins are generated either by hybridomas, B cells or genetically engineered host cells. In the heavy chain, the amino acid sequences run from an N-terminus at the forked ends of the Y configuration to the C-terminus at the bottom of each chain. Those skilled in the art will appreciate that heavy chains are classified as gamma, mu, alpha, delta, or epsilon, (γ, μ, α, δ, ε) with some subclasses among them (e.g., γ1-γ4). It is the nature of this chain that determines the “class” of the antibody as IgG, IgM, IgA IgG, or IgE, respectively. The immunoglobulin subclasses (isotypes) e.g., IgG₁, IgG₂, IgG₃, IgG₄, IgA₁, etc. are well characterized and are known to confer functional specialization. Modified versions of each of these classes and isotypes are readily discernable to the skilled artisan in view of the instant disclosure and, accordingly, are within the scope of the instant invention.

The variable region allows the antibody to selectively recognize and specifically bind epitopes on antigens. That is, the V_{L} domain and Vg domain of an antibody combine to form the variable region that defines a three dimensional antigen binding site. This quaternary antibody structure forms the antigen binding site present at the end of each arm of the Y. More specifically, the antigen binding site is defined by three complementary determining regions (CDRs) on each of the V_{H} and V_{L} chains.

The term “antibody”, as used herein, includes whole antibodies, e.g., of any isotype (IgG, IgA, IgM, IgE, etc.), and includes antigen binding fragments thereof. Exemplary antibodies include monoclonal antibodies, polyclonal antibodies, chimeric antibodies, humanized antibodies, human antibodies, and multivalent antibodies. Antibodies may be fragmented using conventional techniques. Thus, the term antibody includes segments of proteolytically-cleaved or recombinantly-prepared portions of an antibody molecule that are capable of actively binding to a certain antigen. Non-limiting examples of proteolytic and/or recombinant antigen binding fragments include Fab, F(ab′)2, Fab′, Fv, and single chain antibodies (sFv) containing a V[L] and/or V[H] domain joined by a peptide linker.

As used herein, the term “humanized antibody” refers to an antibody or antibody construct in which the complementarity determining regions (CDRs) of an antibody from one species have been grafted onto the framework regions of the variable region of a human. Such antibodies may or may not include framework mutations, backmutations, and/or CDR mutations to optimize antigen binding.

The term “mukispecific” includes binding molecules having specificity for more than one target antigen. Such molecules have more than one binding site where each binding site specifically binds (e.g., immunoreacts with) a different target molecule or a different antigenic site on the same target.

In one embodiment, a mukispecific binding molecule of the invention is a bispecific molecule (e.g., antibody, minibody, domain deleted antibody, or fusion protein) having binding specificity for at least two targets, e.g., more than one target molecule or more than one epitope on the same target molecule.

In one embodiment, modified forms of antibodies can be made from a whole precursor or parent antibody using techniques known in the art. Exemplary techniques are discussed in more detail below. In particularly preferred embodiments both the variable and constant regions of polypeptides of the invention are human. In one embodiment, fully human antibodies can be made using techniques that are known in the art. For example, fully human antibodies against a specific antigen can be prepared by administering the antigen to a transgenic animal which has been modified to produce such antibodies in response to antigenic challenge, but whose endogenous loci have been disabled. Exemplary techniques that can be used to make antibodies are described in U.S. Pat. Nos. 6,150,584; 6,458,592; 6,420,140. Other techniques are known in the art.

In one embodiment, a binding molecule of the invention comprises an antibody molecule, e.g., an intact antibody molecule, or a fragment of an antibody molecule. In another embodiment, binding molecule of the invention is a modified or synthetic antibody molecule. In one embodiment, a binding molecule of the invention comprises all or a portion of (e.g., at least one antigen binding site from, at least one CDR from) a monoclonal antibody, a humanized antibody, a chimeric antibody, or a recombinantly produced antibody.

In embodiments where the binding molecule is an antibody or modified antibody, the antigen binding site and the heavy chain portions need not be derived from the same immunoglobulin molecule. In this regard, the variable region may be derived from any type of animal that can be induced to mount a humoral response and generate immunoglobulins against the desired antigen. As such, the variable region of the polypeptides may be, for example, of mammalian origin e.g., may be human, murine, non-human primate (such as cynomolgus monkeys, macaques, etc.), lupine, camelid (e.g., from camels, llamas and related species). In another embodiment, the variable region may be condricthoid in origin (e.g., from sharks).

In one embodiment, the binding molecules of the invention are modified antibodies. As used herein, the term “modified antibody” includes synthetic forms of antibodies which are altered such that they are not naturally occurring, e.g., antibodies that do not comprise complete heavy chains (such as, domain deleted antibodies or minibodies); multispecific forms of antibodies (e.g., bispecific, trispecific, etc.) altered to bind to two or more different antigens or to different epitopes on a single antigen); heavy chain molecules joined to scFv molecules and the like. ScFv molecules are known in the art and are described, e.g., in U.S. Pat. No. 5,892,019. In addition, the term “modified antibody” includes multivalent forms of antibodies (e.g., trivalent, tetravalent, etc., antibodies that bind to three or more copies of the same antigen).

In one embodiment, the term, “modified antibody” according to the present invention includes immunoglobulins, antibodies, or immunoreactive fragments or recombinants thereof, in which at least a fraction of one or more of the constant region domains has been deleted or otherwise altered so as to provide desired biochemical characteristics such as the ability to non-covalently dimerize, increased ability to localize at the site of a tumor, or reduced serum half-life when compared with a whole, unaltered antibody of approximately the same immunogenicity. In a preferred embodiment, the polypeptides of the present invention are domain deleted antibodies which comprise a polypeptide chain similar to an immunoglobulin heavy chain, but which lack at least a portion of one or more heavy chain domains. More preferably, one entire domain of the constant region of the modified antibody will be deleted and even more preferably all or part of the CH2 domain will be deleted.

In preferred embodiments, an antibody of the invention will not elicit a deleterious immune response in a human. Modifications to the constant region compatible with the instant invention comprise additions, deletions or substitutions of one or more amino acids in one or more domains. That is, the antibodies of the invention may comprise alterations or modifications to one or more of the three heavy chain constant domains (CH1, CH2 or CH3) and/or to the light chain constant region domain (CL).

In one embodiment, the binding molecules of the invention may be modified to reduce their immunogenicity using art-recognized techniques. For example, antibodies or polypeptides of the invention can be humanized, deimmunized, or chimeric antibodies can be made. These types of antibodies are derived from a non-human antibody, typically a murine antibody, that retains or substantially retains the antigen-binding properties of the parent antibody, but which is less immunogenic in humans. This may be achieved by various methods, including (a) grafting the entire non-human variable domains onto human constant regions to generate chimeric antibodies; (b) grafting at least a part of one or more of the non-human complementarity determining regions (CDRs) into a human framework and constant regions with or without retention of critical framework residues; or (c) transplanting the entire non-human variable domains, but “cloaking” them with a human-like section by replacement of surface residues. Such methods are disclosed in Morrison et al., Proc. Natl. Acad. Sci. 81: 6851-5 (1984); Morrison et al., Adv. Immunol. 44: 65-92 (1988); Verhoeyen et al., Science 239: 1534-1536 (1988); Padlan, Molec. Immun. 28: 489-498 (1991); Padlan, Molec. Immun. 31: 169-217 (1994), and U.S. Pat. Nos. 5,585,089, 5,693,761 and 5,693,762 all of which are hereby incorporated by reference in their entirety.

In one embodiment, cells from a subject can be contacted in vitro with the anti-LT-βR activating agent and the at least one additional agent and then introduced into the subject. The subject may then be treated with the second phase of the combination therapy, e.g., the anti-LT-βR activating agent and the at least one additional agent.

The term “combination therapy”, as used herein, refers to a therapeutic regimen comprising, e.g., an anti-LTβR activating agent and at least one second agent, e.g., an agent that inhibits TRAF3 activity, and/or an NFκB1 activating agent. The anti-LTβR activating agent and the at least one second agent may be formulated for separate administration or may be formulated for administration together.

The term “cancer” or “neoplasia” refers in general to any malignant neoplasm or spontaneous growth or proliferation of cells. A subject having “cancer”, for example, may have a leukemia, lymphoma, or other malignancy of blood cells. In certain embodiments, the subject methods are used to treat a tumor (also referred to herein as a “cancerous tumor”), including a solid tumor. Exemplary tumors include but are not limited to non small cell lung cancer (NSCLC), testicular cancer, lung cancer, ovarian cancer, uterine cancer, cervical cancer, pancreatic cancer, colorectal cancer (CRC), breast cancer, as well as prostate, gastric, skin, stomach, esophageal, and bladder cancer. In one embodiment of the invention, a tumor is a colon tumor. In another embodiment of the invention, a tumor is a cervical tumor.

The term “carcinoma” refers to any of various types of malignant neoplasias derived from epithelial cells, e.g., glandular cells (“adenoma” or “adenocarcinoma”) or squamous cells (“squamous cell carcinoma”). Carcinomas often infiltrate into adjacent tissue and spread (“metastasize”) to distant organs, e.g., bone, liver, lung or brain.

The term “chemotherapeutic agent” refers to a molecule or composition used to treat malignancy. Such agents may be used in combination with an anti-LT-βR activating agent or with a combination therapy of the invention. Chemotherapeutic agents include agents that can be conjugated to an anti-LT-βR activating agent and/or a NFκB1 activating agent may be used in combination with the combination therapy in unconjugated form. Exemplary chemotherapeutic agents are discussed below.

The term “effective amount” refers to that amount of combination therapy which is sufficient to affect a desired result on a cancerous cell or tumor, including, but not limited to, for example, reducing tumor size, reducing tumor volume, and/or decreasing vascularization of a solid tumor to an agent, either in vitro or in vivo. In certain embodiments of the invention, an effective amount of a combination therapy is the amount that results in a % tumor inhibition of more than about 58%, 60%, 65%, 70%, 75%, 80%, 85%, 90%, 95%, 100%. The term also includes that amount of a combination therapy which is sufficient to achieve a desired clinical result, including but not limited to, for example, ameliorating'disease, stabilizing a patient, preventing or delaying the development of, or progression of cancer in a patient. An effective amount of the combination therapy can be determined based on one administration or repeated administration. Methods of detection and measurement of the indicators above are known to those of skill in the art. Such methods include, but are not limited to measuring reduction in tumor burden, reduction of tumor size, reduction of tumor volume, reduction in proliferation of secondary tumors, decreased solid tumor vascularization, expression of genes in tumor tissue, presence of biomarkers, lymph node involvement, histologic grade, and nuclear grade.

“Treating cancer” or “treating a subject having cancer” includes inhibition of the replication of cancer cells, inhibition of the spread of cancer, reduction in tumor size, lessening or reducing the number of cancerous cells in the body, and/or amelioration or alleviation of the symptoms of cancer. A treatment is considered therapeutic if there is a decrease in mortality and/or morbidity, and may be performed prophylactically, or therapeutically.

The term “immunotoxin” refers to a hybrid molecule formed by coupling an entire toxin or the A chain of a toxin to a binding molecule. The resulting molecule has the specificity of the binding molecule and has toxicity imparted by the toxin. Such toxins may be conjugated to a binding molecule of the invention. Non-limiting examples of toxins include, e.g., maytansinoids, CC-1065 analogs, calicheamicin derivatives, anthracyclines, vinca alkaloids, ricin, diptheria toxin, and Pseudomonas exotoxin. Exemplary immunotoxic biologic agents include, but are not limited to an anti-CD33 antibody conjugated to calicheamicin, i.e., gemtuzumab ozogamicin, an anti-CD22 variable domain (Fv) fused to truncated Psuedomonas exotoxin, i.e., RFB4(dsFv)-PE38 (BL22), and an interleukin-2 (IL-2) fusion protein comprising diphtheria toxin, i.e., Denileukin diftitox.

A “patient” or “subject” or “host” refers to either a human or non-human animal.

The term “plant alkaloid” refers a compound belonging to a family of alkaline, nitrogen-containing molecules derived from plants that are biologically active and cytotoxic. Examples of plant alkoids include, but are not limited to, taxanes such as docetaxel and paclitaxel and vincas such as vinblastine, vincristine, and vinorelbine. In one embodiment, the plant alkaloid is Taxol.

As used herein, the term “lever” or “amount”, with respect to TRAF3, TRAF2, and/or p53, refers to the expression level, e.g., mRNA level, and/or the protein level, of TRAF3, TRAF2, and/or p53 in a tumor, a cell, or sample. The amount may be either (a) an absolute amount as measured in molecules, moles or weight per unit volume or cells or (b) a relative amount, e.g., measured by densitometric analysis. The amount of TRAF3 and the amount of TRAF2 may be expressed as a direct ratio and is referred to herein as a “TRAF3/TRAF2 ratio”. The amount of TRAF3/TRAF2 may also be described relative to a third marker, e.g.

The amount of TRAF3, TRAF2, and/or p53, and/or the TRAF3/TRAF2 ratio, in a tumor, a cell, or a sample derived from a subject is “altered” (“increased or decreased” or “higher or lower”) relative to a control amount of TRAF3, TRAF2, and/or p53, and/or a control TRAF3/TRAF2 ratio, if the amount of TRAF3, TRAF2, and/or p53, and/or the ratio of TRAF3/TRAF2, is greater or less, respectively, than the control amount and/or ratio by an amount and/or ratio that is greater than the standard error of the assay employed to assess the amount and/or ratio. The amount of TRAF3, TRAF2, and/or p53, and/or the TRAF3/TRAF2 ratio, in a tumor, a cell, or a sample derived from a subject can be considered “higher” or “lower” than the given amount and/or ratio if the difference in the control amount and/or ratio and the sample amount and/or ratio is at least about two, and preferably at least about three, four, or five times, higher or lower, respectively, than the standard error of control and sample measurements of TRAF3, TRAF2, and/or p53, and/or the TRAF3/TRAF2 ratio.

The amount of TRAF3, TRAF2, and/or p53 can be measured by, e.g., by determining the level of expression of TRAF3, e.g., the nucleic acid and/or protein level. In one embodiment, the amount of TRAF3, TRAF2, and/or p53 protein in a tumor, a cell or sample is determined. In another embodiment, the amount of TRAF3, TRAF2, and/or p53 mRNA present in a tumor, a cell, or a sample is determined. These amounts may be used to calculate a TRAF3/TRAF2 ratio, i.e., by dividing the amount of TRAF3 by the amount of TRAF2.

In one embodiment, the amount of TRAF3 and the amount of TRAF2 are normalized to the amount of LTBR. In one embodiment, a resistant cell has a TRAF3/TRAF2 ration of between 1-3 and 1-5, e.g., 1.3, 1.35, 1.4, 1.45, 1.5. In one embodiment, a sensitive cell has a TRAF3/TRAF2 ration of between 0.2-0.4, e.g., 0.2, 0.25, 0.3, 0.35, 0.4.

The term “negative control amount or level” or “normal amount or level” of TRAF3, TRAF2, and/or p53 as used herein, refers to the amount of TRAF3, TRAF2, and/or p53 in a cell or a sample derived from a healthy subject not afflicted with cancer or a cell or a sample derived a portion of the organ afflicted with a tumor that is non-cancerous. Such an amount or level may, for example, be determined by calculating the average amount of TRAF3, TRAF2, and/or p53 present in cells or tissues that are non-cancerous and known to express TRAF3, TRAF2, and/or p53, e.g., express these proteins and/or mRNAs.

Similarly, the term “negative control ratio” or “normal ratio” of TRAF3 to TRAF2, as used herein, refers to the TRAF3/TRAF2 ratio in a cell or a sample derived from a healthy subject not afflicted with cancer, or a cell or a sample derived a portion of the organ afflicted with a tumor that is non-cancerous. Such a ratio may, for example, be determined by calculating the average TRAF3/TRAF2 ratio present in cells or tissues that are non-cancerous and known to express TRAF3 and TRAF2, e.g., express these proteins and/or mRNAs.

As used herein, “known standard” or “control” refers to the amount or level of TRAF3, TRAF2, and/or p53 and/or the TRAF3/TRAF2 ratio. A known standard may be present in, obtained from, or may be a characteristic of a tumor or cell, e.g., a tumor cell, that is sensitive to treatment with an LT-βR activating agent and/or a tumor or cell, e.g., a tumor cell, that is resistant to treatment with an LT-βR activating agent e.g., as empirically demonstrated. Reagents for generating a known standard include, without limitation, tumor cells from a tumor known to be sensitive to treatment with an LT-βR activating agent and tumor cells from a tumor that is resistant to treatment with an LT-βR activating agent. Known standards may also include, be present in, or obtained from tissue culture cell lines, e.g., DLD-1 and WiDr adenocarcinoma cell lines, or tumor xenografts.

A tumor or cells from a tumor that is “sensitive” to treatment with an LT-βR activating agent is a tumor or cell whose growth or replication is inhibited by such a treatment.

A tumor or cells from a tumor that is “resistant” to treatment with an LT-βR activating agent is a tumor or cell whose growth or replication is not inhibited by such a treatment.

An “RNA interfering agent” as used herein, is defined as any agent which interferes with or inhibits expression of a target gene, e.g., a marker of the invention, by RNA interference (RNAi). Such RNA interfering agents include, but are not limited to, nucleic acid molecules including RNA molecules which are homologous to the target gene, e.g., a marker of the invention, or a fragment thereof, short interfering RNA (siRNA), and small molecules which interfere with or inhibit expression of a target gene by RNA interference (RNAi).

“RNA interference (RNAi)” is an evolutionally conserved process whereby the expression or introduction of RNA of a sequence that is identical or highly similar to a target gene results in the sequence specific degradation or specific post-transcriptional gene silencing (PTGS) of messenger RNA (mRNA) transcribed from that targeted gene (see Coburn, G. and Cullen, B. (2002) J. of Virology 76(18):9225), thereby inhibiting expression of the target gene. In one embodiment, the RNA is double stranded RNA (dsRNA). This process has been described in plants, invertebrates, and mammalian cells. In nature, RNAi is initiated by the dsRNA-specific endonuclease Dicer, which promotes processive cleavage of long dsRNA into double-stranded fragments termed siRNAs. siRNAs are incorporated into a protein complex that recognizes and cleaves target mRNAs. RNAi can also be initiated by introducing nucleic acid molecules, e.g., synthetic siRNAs or RNA interfering agents, to inhibit or silence the expression of target genes. As used herein, “inhibition of target gene expression” or “inhibition of marker gene expression” includes any decrease in expression or protein activity or level of the target gene (e.g., a marker gene of the invention) or protein encoded by the target gene, e.g., a marker protein of the invention. The decrease may be of at least 30%, 40%, 50%, 60%, 70%, 80%, 90%, 95% or 99% or more as compared to the expression of a target gene or the activity or level of the protein encoded by a target gene which has not been targeted by an RNA interfering agent.

“Short interfering RNA” (siRNA), also referred to herein as “small interfering RNA” is defined as an agent which functions to inhibit expression of a target gene, e.g., by RNAi. An siRNA may be chemically synthesized, may be produced by in vitro transcription, or may be produced within a host cell. In one embodiment, siRNA is a double stranded RNA (dsRNA) molecule of about 15 to about 40 nucleotides in length, preferably about 15 to about 28 nucleotides, more preferably about 19 to about 25 nucleotides in length, and more preferably about 19, 20, 21, or 22 nucleotides in length, and may contain a 3′ and/or 5′ overhang on each strand having a length of about 0, 1, 2, 3, 4, or 5 nucleotides. The length of the overhang is independent between the two strands, i.e., the length of the over hang on one strand is not dependent on the length of the overhang on the second strand. Preferably the siRNA is capable of promoting RNA interference through degradation or specific post-transcriptional gene silencing (PTGS) of the target messenger RNA (mRNA).

In another embodiment, an siRNA is a small hairpin (also called stem loop) RNA (shRNA). In one embodiment, these shRNAs are composed of a short (e.g., 19-25 nucleotide) antisense strand, followed by a 5-9 nucleotide loop, and the analogous sense strand. Alternatively, the sense strand may precede the nucleotide bop structure and the antisense strand may follow. These shRNAs may be contained in plasmids, retroviruses, and lentiviruses and expressed from, for example, the pol III U6 promoter, or another promoter (see, e.g., Stewart, et al. (2003) RNA April; 9(4):493-501 incorporated be reference herein).

RNA interfering agents, e.g., siRNA molecules, may be administered to a patient having or at risk for having cancer, to inhibit expression of a marker gene of the invention, e.g., a marker gene which is overexpressed in cancer (such as the markers listed in Table 2) and thereby treat, prevent, or inhibit cancer in the subject.

“Complementary” refers to the broad concept of sequence complementarity between regions of two nucleic acid strands or between two regions of the same nucleic acid strand. It is known that an adenine residue of a first nucleic acid region is capable of forming specific hydrogen bonds (“base pairing”) with a residue of a second nucleic acid region which is antiparallel to the first region if the residue is thymine or uracil. Similarly, it is known that a cytosine residue of a first nucleic acid strand is capable of base pairing with a residue of a second nucleic acid strand which is antiparallel to the first strand if the residue is guanine. A first region of a nucleic acid is complementary to a second region of the same or a different nucleic acid if, when the two regions are arranged in an antiparallel fashion, at least one nucleotide residue of the fust region is capable of base pairing with a residue of the second region. Preferably, the fust region comprises a first portion and the second region comprises a second portion, whereby, when the first and second portions are arranged in an antiparallel fashion, at least about 50%, and preferably at least about 75%, at least about 90%, or at least about 95% of the nucleotide residues of the first portion are capable of base pairing with nucleotide residues in the second portion. More preferably, all nucleotide residues of the first portion are capable of base pairing with nucleotide residues in the second portion.

The terms “homology” or “identity,” as used interchangeably herein, refer to sequence similarity between two polynucleotide sequences or between two polypeptide sequences, with identity being a more strict comparison. The phrases “percent identity or homology” and “% identity or homology” refer to the percentage of sequence similarity found in a comparison of two or more polynucleotide sequences or two or more polypeptide sequences. “Sequence similarity” refers to the percent similarity in base pair sequence (as determined by any suitable method) between two or more polynucleotide sequences. Two or more sequences can be anywhere from 0-100% similar, or any integer value there between. Identity or similarity can be determined by comparing a position in each sequence that may be aligned for purposes of comparison. When a position in the compared sequence is occupied by the same nucleotide base or amino acid, then the molecules are identical at that position. A degree of similarity or identity between polynucleotide sequences is a function of the number of identical or matching nucleotides at positions shared by the polynucleotide sequences. A degree of identity of polypeptide sequences is a function of the number of identical amino acids at positions shared by the polypeptide sequences. A degree of homology or similarity of polypeptide sequences is a function of the number of amino acids at positions shared by the polypeptide sequences. The term “substantial homology,” as used herein, refers to homology of at least 50%, more preferably, 60%, 70%, 80%, 90%, 95% or more.

As used herein, an “isolated” nucleic acid molecule is one which is separated from other nucleic acid molecules which are present in the natural source of the nucleic acid. For example, with regards to genomic DNA, the term “isolated” includes nucleic acid molecules which are separated from the chromosome with which the genomic DNA is naturally associated. Preferably, an “isolated” nucleic acid molecule is free of sequences which naturally flank the nucleic acid molecule (i.e., sequences located at the 5′ and 3′ ends of the nucleic acid molecule) in the genomic DNA of the organism from which the nucleic acid molecule is derived.

As used herein, an “isolated protein” or “isolated polypeptide” refers to a protein or polypeptide that is substantially free of other proteins, polypeptides, cellular material and culture medium when isolated from cells or produced by recombinant DNA techniques, or chemical precursors or other chemicals when chemically synthesized. An “isolated” or “purified” protein or biologically active portion thereof is substantially free of cellular material or other contaminating proteins from the cell or tissue source from which the protein is derived, or substantially free from chemical precursors or other chemicals when chemically synthesized. The language “substantially free of cellular material” includes preparations of protein in which the protein is separated from cellular components of the cells from which it is isolated or recombinantly produced.

The nucleic acids of the invention can be prepared, e.g., by standard recombinant DNA techniques. A nucleic acid of the invention can also be chemically synthesized using standard techniques. Various methods of chemically synthesizing polydeoxynucleotides are known, including solid-phase synthesis which has been automated in commercially available DNA synthesizers (See e.g., Itakura et al. U.S. Pat. No. 4,598,049; Caruthers et al. U.S. Pat. No. 4,458,066; and Itakura U.S. Pat. Nos. 4,401,796 and 4,373,071, incorporated by reference herein).

As used herein, the term “vector” refers to a nucleic acid molecule capable of transporting another nucleic acid to which it has been linked. One type of vector is a “plasmid”, which refers to a circular double stranded DNA loop into which additional DNA segments may be ligated. Another type of vector is a viral vector, wherein additional DNA segments may be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors are capable of directing the expression of genes to which they are operatively linked. Such vectors are referred to herein as “recombinant expression vectors” or simply “expression vectors”. In general, expression vectors of utility in recombinant DNA techniques are often in the form of plasmids. In the present specification, “plasmid” and “vector” may be used interchangeably as the plasmid is the most commonly used form of vector. However, the invention is intended to include such other forms of expression vectors, such as viral vectors (e.g., replication defective retroviruses, adenoviruses and adeno-associated viruses), which serve equivalent functions.

As used herein, the term “host cell” is intended to refer to a cell into which a nucleic acid molecule of the invention, such as a recombinant expression vector of the invention, has been introduced. The terms “host cell” and “recombinant host cell” are used interchangeably herein. It should be understood that such terms refer not only to the particular subject cell but to the progeny or potential progeny of such a cell. Because certain modifications may occur in succeeding generations due to either mutation or environmental influences, such progeny may not, in fact, be identical to the parent cell, but are still included within the scope of the term as used herein. Preferably a host cell is a mammalian cell, e.g., a human cell.

As used herein, a “naturally-occurring” nucleic acid molecule refers to an RNA or DNA molecule having a nucleotide sequence that occurs in nature (e.g. encodes a natural protein).

The term “probe” refers to any molecule that is capable of selectively binding to TRAF3, TRAF2, and/or p53, for example, a TRAF3, TRAF2, and/or p53 nucleotide transcript or TRAF3, TRAF2, and/or p53 protein. Probes can be synthesized by one of skill in the art, or derived from appropriate biological preparations. Probes may be specifically designed to be labeled. Examples of molecules that can be utilized as probes include, but are not limited to, RNA, DNA, proteins, antibodies, and organic molecules.

Cancer is “inhibited” if at least one symptom of the cancer is alleviated, terminated, slowed, or prevented. As used herein, cancer is also “inhibited” if recurrence or metastasis of the cancer is reduced, slowed, delayed, or prevented.

A kit is any manufacture (e.g. a package or container) comprising at least one reagent, e.g. a probe, for specifically detecting a marker of the invention, the manufacture being promoted, distributed, or sold as a unit for performing the methods of the present invention.

### 2. Methods of the Invention

The present invention provides novel methods for predicting the sensitivity or resistance of a tumor to treatment with an LT-β-R activating agent. The present invention also provides a method for determining whether LT-β-R signaling is activated in a sample, e.g., tissue or a cell(s).

In one embodiment, these methods generally comprise comparing the ratio of the amount of TRAF3 to the amount of TRAF2 to determine the susceptibility of a tumor to an LT-β-R activating agent or to determine the status of LT-β-R signaling, e.g., active or inactive, in a cell or tissue sample. In one embodiment, the method of the invention includes determining the level of expression of TRAF3 and TRAF2, e.g., the nucleic acid or protein level, present in or obtained from a sample of the tumor, e.g., prior to administration of the treatment, and dividing the amount of TRAF3 by the amount of TRAF2 to obtain the TRAF3/TRAF2 ratio, with a known standard ratio of the amount of TRAF3 to the amount of TRAF2 present in or obtained from a tumor with known sensitivity or a known standard ratio of the amount of TRAF3 to the amount of TRAF2 present in or obtained from a tumor with known resistance to treatment with an LT-β-R activating agent, evaluating the TRAF3/TRAF2 ratio in the sample relative to the known standard ratios, thereby predicting the sensitivity or resistance of the tumor to treatment with an LT-β-R activating agent. In one embodiment, the tumor is predicted to be sensitive to the LT-β-R activating agent if the TRAF3/TRAF2 ratio obtained from the sample of the tumor prior to administration of the treatment is about equal to or less than the TRAF3/TRAF2 ratio obtained from a tumor with known sensitivity to treatment with the LT-β-R activating agent. In another embodiment, the tumor is predicted to be sensitive to the LT-β-R activating agent if the TRAF3/TRAF2 ratio obtained from the sample of the tumor prior to administration of the treatment is less than the TRAF3/TRAF2 ratio obtained from a tumor with known resistance to treatment with the LT-β-R activating agent. In another embodiment, the tumor is predicted to be resistant to the LT-β-R activating agent if the TRAF3/TRAF2 ratio obtained from the sample of the tumor prior to administration of the treatment is greater than the TRAF3/TRAF2 ratio obtained from a tumor with known sensitivity to treatment with the LT-β-R activating agent. In yet another embodiment, the tumor is predicted to be resistant to the LT-β-R activating agent if the TRAF3/TRAF2 ratio obtained from the sample of the tumor prior to administration of the treatment is about equal to or greater than the TRAF3/TRAF2 ratio obtained from a tumor with known resistance to treatment with the LT-β-R activating agent.

In another embodiment, these methods generally comprise comparing the amount of TRAF3, e.g., by determining the level of expression of TRAF3, e.g., the nucleic acid or protein level, present in or obtained from a sample of the tumor prior to administration of the treatment with a known standard amount of TRAF3 present in or obtained from a tumor with known sensitivity or a known standard amount of TRAF3 present in or obtained from a tumor with known resistance to treatment with an LT-β-R activating agent, evaluating the amount of TRAF3 in the sample relative to the known standard amounts, thereby predicting the sensitivity or resistance of the tumor to treatment with an LT-β-R activating agent. In one embodiment, the tumor is predicted to be sensitive to the LT-β-R activating agent if the amount of TRAF3 present in or obtained from the sample of the tumor prior to administration of the treatment is about equal to or less than the known standard amount of TRAF3 present in or obtained from a tumor with known sensitivity to treatment with the LT-β-R activating agent. In another embodiment, the tumor is predicted to be sensitive to the LT-β-R activating agent if the amount of TRAF3 present in or obtained from the sample of the tumor prior to administration of the treatment is less than the known standard amount of TRAF3 present in or obtained from a tumor with known resistance to treatment with the LT-β-R activating agent. In another embodiment, the tumor is predicted to be resistant to the LT-Q-R activating agent if the amount of TRAF3 present in or obtained from the sample of the tumor prior to administration of the treatment is greater than the known standard amount of TRAF3 present in or obtained from a tumor with known sensitivity to treatment with the LT-β-R activating agent. In yet another embodiment, the tumor is predicted to be resistant to the LT-β-R activating agent if the amount of TRAF3 obtained from the sample of the tumor prior to administration of the treatment is about equal to or greater than the known standard amount of TRAF3 present in or obtained from a tumor with known resistance to treatment with the LT-β-R activating agent.

The invention also provides methods for predicting the efficacy of a treatment regimen, e.g., a treatment comprising administration of a lymphotoxin-0 receptor (LT-β-R) activating agent to a subject having cancer.

In one embodiment, the methods generally comprise analyzing the ratio of the amount of TRAF3 to the amount of TRAF2 present in a tumor or tumor cell, e.g., obtaining a tumor tissue sample from the subject having cancer, wherein the sample is obtained prior to administration of the treatment, measuring the ratio of the amount of TRAF3 to the amount of TRAF2, e.g., by determining the level of expression of TRAF3 and TRAF2, e.g., the nucleic acid or protein level, in the tissue sample, and dividing the amount of TRAF3 by the amount of TRAF2 to obtain the TRAF3/TRAF2 ratio, and comparing the foregoing ratio with the normal TRAF3/TRAF2 ratio, wherein a TRAF3/TRAF2 ratio in the tissue sample approximately equal to or less than the normal amount predicts that the treatment will be efficacious for the treatment of cancer and TRAF3/TRAF2 ratio in the tissue sample greater than the normal amount predicts that the treatment will not be efficacious for the treatment of cancer.

In another embodiment of the invention, the methods generally comprise analyzing the amount of TRAF3 present in a tumor or tumor cell, e.g., obtaining a tumor tissue sample from the subject having cancer, wherein the sample is obtained prior to administration of the treatment, measuring the amount of TRAF3, e.g., by determining the level of expression of TRAF3, e.g., the nucleic acid or protein level, in the tissue sample, and comparing the foregoing amount with the normal amount of TRAF3, wherein an amount of TRAF3 in the tissue sample approximately equal to or less than the normal amount predicts that the treatment will be efficacious for the treatment of cancer and an amount of TRAF3 in the tissue sample greater than the normal amount predicts that the treatment will not be efficacious for the treatment of cancer.

The invention also provides methods for predicting the efficacy of a treatment comprising administration of a lymphotoxin-β receptor (LT-β-R) activating agent to a subject having cancer, said method comprising, analyzing the amount of TRAF2 present in a tumor or tumor cell, e.g., obtaining a tumor tissue sample from the subject having cancer, wherein the sample is obtained prior to administration of the treatment, measuring the amount of TRAF2, e.g., by determining the level of expression of TRAF2, e.g., the nucleic acid or protein level, in the tissue sample, and comparing the foregoing amount with the normal amount of TRAF2, wherein an amount of TRAF2 in the tissue sample approximately equal to or less than the normal amount predicts that the treatment will be efficacious for the treatment of cancer and an amount of TRAF2 in the tissue sample greater than the normal amount predicts that the treatment will not be efficacious for the treatment of cancer.

The invention further provides methods for predicting the efficacy of a treatment comprising administration of a lymphotoxin-β receptor (LT-β-R) activating agent to a subject having cancer. The method comprises analyzing the amount of p53 in a tumor or tumor cell, e.g., obtaining a tumor tissue sample from a patient having cancer, wherein the sample is obtained prior to the treatment, measuring the amount of p53 in the tissue sample, and comparing the amount of p53 in the tissue sample with the normal amount of p53, thereby predicting the efficacy of the treatment.

The invention also provides a method for predicting whether treatment of a tumor with an LT-β-R activating agent will be efficacious, comprising determining the amount of p53 present in a sample of the tumor, wherein a significantly higher amount of p53 in the tissue sample relative to the normal amount of p53 predicts that the treatment of the tumor with an LT-β-R activating agent will be efficacious.

The invention also provides a method for determining whether a subject having a tumor is a candidate for treatment with an LT-β-R activating agent, the method comprising, determining the amount of p53 present in a sample of the tumor, and comparing the amount of p53 present in the sample to a normal amount of p53, thereby determining whether the subject having the tumor is a candidate for treatment with an LT-β-R activating agent.

The prognostic methods of the present invention can be practiced in conjunction with any other method used by the skilled practitioner to prognose the efficacy of treatment with a therapy as described herein, prognose the sensitivity or resistance of a tumor to treatment as described herein, and/or determine whether a subject having a tumor is a candidate for treatment as described herein. For example, the methods of the invention may be performed in conjunction with a morphological or cytological analysis of the sample obtained from the subject. Exemplary morphological analyses may include, for example, lymph node involvement, histologic grade, and/or nuclear grade. Cytological methods may include immunohistochemical or immunofluorescence detection (and quantitation if appropriate) of any other molecular marker either by itself, in conjunction with other markers, and/or in conjunction with TRAF3, TRAF2, and/or p53. Other methods would include detection of other markers by in situ PCR, or by extracting tissue and quantitating other markers by real time PCR. PCR is defined as polymerase chain reaction.

In general, it is preferable that the difference between the amount of TRAF3, TRAF2, and/or p53 and/or the TRAF3/TRAF2 ratio in a sample from a subject having cancer or in a tumor and the amount of TRAF3, TRAF2, and/or p53 and/or the TRAF3/TRAF2 ratio in the control or reference sample, is as great as possible. Although this difference can be as small as the limit of detection of the method for determining the amount and/or ratio it is preferred that the difference be at least greater than the standard error of the assessment method, and preferably a difference of at least 2-, 3-, 4-, 5-, 6-, 7-, 8-, 9-, 10-, 15-, 20-, 25-, 100-, 500-, 1000-fold or greater than the standard error of the assessment method.

An alteration in the amount of TRAF3, TRAF2, and/or p53 and/or the TRAF3/TRAF2 ratio in normal (e.g., non-cancerous and/or prior to treatment) tissue can be assessed in a variety of ways. In one embodiment, the amount and/or ratio is assessed by assessing the expression level of TRAF3, TRAF2, and/or p53 and/or ratio in cells which appear to be non-cancerous and by comparing the foregoing normal level of TRAF3, TRAF2, and/or p53 and/or the normal TRAF3/TRAF2 ratio with the expression level of TRAF3, TRAF2, and/or p53 and/or the TRAF3/TRAF2 ratio in the cells which are suspected of being cancerous. For example, when laparoscopy or other medical procedure, reveals the presence of a tumor on one portion of an organ, the normal expression level may be assessed using the non-affected portion of the organ, and this normal level may be compared with the level of TRAF3, TRAF2, and/or p53 and/or the TRAF3/TRAF2 ratio in an affected portion (i.e., the tumor) of the organ. In another embodiment, the amount and/or ratio is assessed by assessing the expression level of TRAF3, TRAF2, and/or p53 and/or the TRAF3/TRAF2 ratio in cells from a sample and by comparing the foregoing level of TRAF3, TRAF2, and/or p53 and/or the TRAF3/TRAF2 ratio with the expression level of TRAF3, TRAF2, and/or p53 and/or the TRAF3/TRAF2 ratio in cells from a tumor known to be resistant to treatment with an LT-β-R activating agent and/or with the expression level of TRAF3, TRAF2, and/or p53 and/or the TRAF3/TRAF2 ratio in cells from a tumor known to be sensitive to treatment with an LT-β-R activating agent.

Alternatively, and particularly as further information becomes available as a result of routine performance of the methods described herein, population-average values for “normal” levels, “resistant” levels, and/or “sensitive” levels of TRAM, TRAF2, and/or p53 and/or the TRAF3/TRAF2 ratio may be used. In other embodiments, the “normal” level of TRAF3, TRAF2, and/or p53 and/or the TRAF3/TRAF2 ratio may be determined by assessing level of TRAF3, TRAF2, and/or p53 and/or the TRAF3/TRAF2 ratio present in a subject sample obtained from a non-cancer-afflicted subject, from a subject sample obtained from a subject before the suspected onset of cancer in the subject, from archived subject samples, and the like. Such a non-cancerous amount and/or ratio may be used as a negative control in determining whether the amount of TRAF3, TRAF2, and/or p53 and/or the TRAF3/TRAF2 ratio present in a tumor is relatively high or low. In a preferred embodiment, the level of TRAF3, TRAF2, and/or p53 and/or the TRAF3/TRAF2 ratio is determined as described above.

As described herein, tumors that are resistant to treatment with an LT-β-R activating agent have an increased amount of TRAF3 and a decreased amount of NFκB1 and tumors that are sensitive to treatment with an LT-β-R activating agent have a decreased amount of TRAF3 and an increased amount of NFκB1. While, some of these changes result from the occurrence of the cancer, these changes may also induce, maintain, and promote the cancerous state. Thus, cancer may be treated by increasing the expression and/or activity of LT-β-R combined with increasing the expression and/or activity of NFκB1 and/or decreasing the expression and/or activity of TRAF3.

Accordingly, another aspect of the invention pertains to therapeutic methods for treating a subject suffering from cancer. In one aspect the invention provides a method for treating a subject having a cancerous tumor comprising administering to the subject an LT-β-R activating agent and an agent that inhibits TRAF3 activity. In another aspect, the invention provides a method for treating a subject having a cancerous tumor comprising administering to the subject an LT-β-R activating agent and an NFκB1 activating agent. In yet another aspect, the invention provides a method for increasing the efficacy of treatment of a subject having a tumor with a lymphotoxin-β receptor (LT-β-R) activating agent, comprising administering to the subject, an agent which inhibits TRAF3 activity, such that the efficacy of treatment of the tumor with the LT-β-R activating agent is increased.

The methods generally involve administering to the subject a combination therapy. In certain embodiments of the invention, the methods may further comprise administering to the subject a chemotherapeutic agent.

In one embodiment, the agent that inhibits TRAF3 activity is selected from the group consisting of an antibody, an siRNA molecule, and an antisense nucleic acid molecule.

Exemplary, non-limiting siRNA molecules specific for TRAF3 are shown below and in the appended Examples:

Beginning at nucleotide 216 of GenBank accession No.: gi:22027617:

Sense strand siRNA: AGAAGUGAUGCCACUUGGUUU
Antisense strand siRNA: ACCAAGUGGCAUCACUUCUUU

Beginning at nucleotide 501 of GenBank accession No.: gi:22027617:

Sense strand siRNA: GUACAAGUGUGAGAAGUGCUU
Antisense strand siRNA: GCACUUCUCACACUUGUACUU

Beginning at nucleotide 819 of GenBank accession No.: gi:22027617:

Sense strand siRNA: GGUCUUGAGGAAAGACCUGUU
Antisense strand siRNA: CAGGUCUUUCCUCAAGACCUU

Beginning at nucleotide 1596 of GenBank accession No.: gi:22027617:

Sense strand siRNA: UGGAGUGCUCAUCUGGAAGUU
Antisense strand siRNA: CUUCCAGAUGAGCACUCCAUU

Beginning at nucleotide 2094 of GenBank accession No.: gi:22027617:

Sense strand siRNA: CUCCUCUGGGGGAUUUGAAUU
Antisense strand siRNA: UUCAAAUCCCCCAGAGGAGUU

The methods of the present invention may be used to treat cancers, including but not limited to treating solid tumors, e.g., a carcinoma. Examples of solid tumors, e.g., carcinomas, that can be treated by compounds of the present invention, include but are not limited to breast, testicular, lung, ovary, uterine, cervical, pancreatic, non small cell lung (NSCLC), colon, as well as prostate, gastric, skin, stomach, esophagus and bladder cancer. In one embodiment, the tumor is a colon tumor. In one embodiment, the tumor is a cervical tumor. In another embodiment, the tumor is a colon tumor or a cervical tumor.

In certain embodiments, the method comprises parenterally administering an effective amount of an anti-LT-β-R activating agent and at least one additional agent, i.e., an NFκB1 activating agent or a TRAF3 inhibitory agent, to a subject. In one embodiment, the method comprises intraarterial administration of an anti-LT-β-R activating agent and at least one additional agent to a subject. In other embodiments, the method comprises administering an effective amount of an anti-LT-β-R activating agent and at least one additional agent directly to the arterial blood supply of a tumor in a subject. In one embodiment, the methods comprise administering an effective amount of an anti-LT-β-R activating agent and at least one additional agent directly to the arterial blood supply of the cancerous tumor using a catheter. In embodiments where a catheter is used to administer an anti-LT-β-R activating agent and at least one additional agent, the insertion of the catheter may be guided or observed by fluoroscopy or other method known in the art by which catheter insertion may be observed and/or guided. In another embodiment, the method comprises chemoembolization. For example a chemoembolization method may comprise blocking a vessel feeding the cancerous tumor with a composition comprised of a resin-like material mixed with an oil base (e.g., polyvinyl alcohol in Ethiodol) and one or more biologic agents. In still other embodiments, the method comprises systemic administration of an anti-LT-β-R activating agent and at least one additional agent to a subject.

In general, chemoembolization or direct intraarterial or intravenous injection therapy utilizing pharmaceutical compositions of the present invention is typically performed in a similar manner, regardless of the site. Briefly, angiography (a road map of the blood vessels), or more specifically in certain embodiments, arteriography, of the area to be embolized may be first performed by injecting radiopaque contrast through a catheter inserted into an artery or vein (depending on the site to be embolized or injected) as an X-ray is taken. The catheter may be inserted either percutaneously or by surgery. The blood vessel may be then embolized by refluxing pharmaceutical compositions of the present invention through the catheter, until flow is observed to cease. Occlusion may be confirmed by repeating the angiogram. In embodiments where direct injection is used, the blood vessel is then infused with a pharmaceutical composition of the invention in the desired dose.

Embolization therapy generally results in the distribution of compositions containing inhibitors throughout the interstices of the tumor or vascular mass to be treated. The physical bulk of the embolic particles clogging the arterial lumen results in the occlusion of the blood supply. In addition to this effect, the presence of an anti-angiogenic factor(s) prevents the formation of new blood vessels to supply the tumor or vascular mass, enhancing the devitalizing effect of cutting off the blood supply. Direct intrarterial or intravenous generally results in distribution of compositions containing inhibitors throughout the interstices of the tumor or vascular mass to be treated as well. However, the blood supply is not generally expected to become occluded with this method.

In one aspect of the present invention, primary and secondary tumors of the liver or other tissues may be treated utilizing embolization or direct intraarterial or intravenous injection therapy. Briefly, a catheter is inserted via the femoral or brachial artery and advanced into the hepatic artery by steering it through the arterial system under fluoroscopic guidance. The catheter is advanced into the hepatic arterial tree as far as necessary to allow complete blockage of the blood vessels supplying the tumor(s), while sparing as many of the arterial branches supplying normal structures as possible. Ideally this will be a segmental branch of the hepatic artery, but it could be that the entire hepatic artery distal to the origin of the gastroduodenal artery, or even multiple separate arteries, will need to be blocked depending on the extent of tumor and its individual blood supply. Once the desired catheter position is achieved, the artery is embolized by injecting compositions (as described above) through the arterial catheter until flow in the artery to be blocked ceases, preferably even after observation for 5 minutes. Occlusion of the artery may be confirmed by injecting radio-opaque contrast through the catheter and demonstrating by fluoroscopy or X-ray film that the vessel which previously filled with contrast no longer does so. In embodiments where direct injection is used, the artery is infused by injecting compositions (as described above) through the arterial catheter in a desired dose. The same procedure may be repeated with each feeding artery to be occluded.

In most embodiments, the combination therapy will incorporate the substance or substances to be delivered in an amount sufficient to deliver to a patient a therapeutically effective amount of an incorporated therapeutic agent or other material as part of a prophylactic or therapeutic treatment. The desired concentration of active compound in the particle will depend on absorption, inactivation, and excretion rates of the drug as well as the delivery rate of the compound. It is to be noted that dosage values may also vary with the severity of the condition to be alleviated. It is to be further understood that for any particular subject, specific dosage regimens should be adjusted over time according to the individual need and the professional judgment of the person administering or supervising the administration of the compositions. Typically, dosing will be determined using techniques known to one skilled in the art. The selected dosage level will depend upon a variety of factors including the activity of the particular compound of the present invention employed, or the ester, salt or amide thereof, the route of administration, the time of administration, the rate of excretion or metabolism of the particular compound being employed, the duration of the treatment, other drugs, compounds and/or materials used in combination with the particular compound employed, the age, sex, weight, condition, general health and prior medical history of the patient being treated, and like factors well known in the medical arts.

Dosage may be based on the amount of the composition per kg body weight of the patient. Other amounts will be known to those of skill in the art and readily determined. Alternatively, the dosage of the subject invention may be determined by reference to the plasma concentrations of the composition. For example, the maximum plasma concentration (Cmax) and the area under the plasma concentration-time curve from time 0 to infinity (AUC (0-4)) may be used. Dosages for the present invention include those that produce the above values for Cmax and AUC (0-4) and other dosages resulting in larger or smaller values for those parameters.

A physician or veterinarian having ordinary skill in the art can readily determine and prescribe the effective amount of the pharmaceutical composition required. For example, the physician or veterinarian could start doses of the compounds of the invention employed in the pharmaceutical composition at levels lower than that required in order to achieve the desired therapeutic effect and gradually increase the dosage until the desired effect is achieved.

In general, a suitable daily dose of a combination therapy of an anti-LT-β-R activating agent and at least one additional agent will be that amount of the combination therapy which is the lowest dose effective to produce a therapeutic effect. Such an effective dose will generally depend upon the factors described above.

In one embodiment, the effective dose of each agent in the combination therapy of the invention is the dose shown to be effective for that agent alone. In one embodiment, the effective dose of the anti-LT-β-R activating agent is about 16 mg/m².

In another embodiment, the effective dose of the anti-LT-β-R activating agent is about 20 mg/m².

In another embodiment, the effective dose of one or more agents in the combination therapy is a lower dose than that shown to be effective for each agent alone.

The precise time of administration and amount of any particular compound that will yield the most effective treatment in a given patient will depend upon the activity, pharmacokinetics, and bioavailability of a particular compound, physiological condition of the patient (including age, sex, disease type and stage, general physical condition, responsiveness to a given dosage and type of medication), route of administration, and the like. The guidelines presented herein may be used to optimize the treatment, e.g., determining the optimum time and/or amount of administration, which will require no more than routine experimentation consisting of monitoring the subject and adjusting the dosage and/or timing

While the subject is being treated, the health of the patient may be monitored by measuring one or more of the relevant indices at predetermined times during a 24-hour period. Treatment, including supplement, amounts, times of administration and formulation, may be optimized according to the results of such monitoring. The patient may be periodically reevaluated to determine the extent of improvement by measuring the same parameters, the first such reevaluation typically occurring at the end of four weeks from the onset of therapy, and subsequent reevaluations occurring every four to eight weeks during therapy and then every three months thereafter. Therapy may continue for several months or even years, with a minimum of one month being a typical length of therapy for humans. Adjustments to the amounts) of agent administered and possibly to the time of administration may be made based on these reevaluations.

Treatment may be initiated with smaller dosages which are less than the optimum dose of the compound. Thereafter, the dosage may be increased by small increments until the optimum therapeutic effect is attained.

Knowing this helps oncologists decide which drugs are likely to work well together and, if more than one drug will be used, plan exactly when each of the drugs should be given (in which order and how often).

In one embodiment of the invention, chemotherapeutic agents are further used in the combination treatment of the invention. Examples of chemotherapeutic agents which may be used include, but are not limited to the following: platinums (i.e., cis platinum), anthracyclines, nucleoside analogs (purine and pyrimidine), taxanes, camptothecins, epipodophyllotoxins, DNA alkylating agents, folate antagonists, vinca alkaloids, ribonucleotide reductase inhibitors, estrogen inhibitors, progesterone inhibitors, androgen inhibitors, aromatase inhibitors, interferons, interleukins, monoclonal antibodies, taxol, camptosar, adriamycin (dox), 5-FU and gemcitabine. Such chemotherapeutic agents may be employed in the practice of the invention by coadministration of the combination therapy and the chemotherapeutic. In one embodiment, an anti-LT-βR activating agent is administered in combination with at least one additional agent and a chemotherapeutic agent selected from the group consisting of gemcitabine, adriamycin, Camptosar, carboplatin, cisplatin, and Taxol. Methods for treating cancer comprising administering an anti-lymphotoxin-beta receptor (LT-β-R) activating agent and at least one chemotherapeutic agent are also described in U.S. appln. Ser. No. 11/156,109, incorporated by reference herein.

In one embodiment, an anti-LT-β-R activating agent is conjugated to a chemotherapeutic agent. In one embodiment, an anti-LT-β-R activating agent is nonconjugated to a chemotherapeutic agent.

The combined use of an anti-LT-β-R activating agent and at least one additional agent as described herein (optionally in combination with other chemotherapeutics), may reduce the required dosage for any individual component, e.g., if the onset and duration of effect of the different components may be complimentary. In such combined therapy, the different active agents may be delivered together or separately, and simultaneously or at different times within the day. Toxicity and therapeutic efficacy of subject compounds may be determined by standard pharmaceutical procedures in cell cultures or experimental animals, e.g., for determining the LD50 and the ED50. Compositions that exhibit large therapeutic indices are preferred. Although compounds that exhibit toxic side effects may be used, care should be taken to design a delivery system that targets the compounds to the desired site in order to reduce side effects.

The data obtained from the cell culture assays and animal studies may be used in formulating a range of dosage for use in humans. The dosage of any supplement, or alternatively of any components therein, lies preferably within a range of circulating concentrations that include the ED50 with little or no toxicity. The dosage may vary within this range depending upon the dosage form employed and the route of administration utilized. For agents of the present invention, the therapeutically effective dose may be estimated initially from cell culture assays. A dose may be formulated in animal models to achieve a circulating plasma concentration range that includes the IC50 (i.e., the concentration of the test compound which achieves a half-maximal inhibition of symptoms) as determined in cell culture. Such information may be used to more accurately determine useful doses in humans. Levels in plasma may be measured, for example, by high performance liquid chromatography.

For activating or inhibitory agents that comprise nucleic acids (including recombinant expression vectors encoding T-bet polypeptide, antisense RNA), the agents can be introduced into cells of the subject using methods known in the art for introducing nucleic acid (e.g., DNA) into cells. Examples of such methods are described below.

In the methods of the invention in which the at least one additional agent is an antisense nucleic acid molecule, administration to a subject or generation of is typically in situ such that the antisense nucleic acid molecules hybridize with or bind to cellular mRNA and/or genomic DNA thereby inhibit expression of the protein, e.g., by inhibiting transcription and/or translation. The hybridization can be by conventional nucleotide complementarity to form a stable duplex, or, for example, in the case of an antisense nucleic acid molecule which binds to DNA duplexes, though specific interactions in the major groove of the double helix. An example of a route of administration of antisense nucleic acid molecules of the invention includes direct injection at a tissue site. Alternatively, antisense nucleic acid molecules can be modified to target selected cells and then administered systemically. For example, for systemic administration, antisense molecules can be modified such that they specifically bind to receptors or antigens expressed on a selected cell surface, e.g., by linking the antisense nucleic acid molecules to peptides or antibodies which bind to cell surface receptors or antigens. The antisense nucleic acid molecules can also be delivered to cells using the vectors known to one of skill in the art. To achieve sufficient intracellular concentrations of the antisense molecules, vector constructs in which the antisense nucleic acid molecule is placed under the control of a strong pol II or pol III promoter are preferred.

The administration of a nucleic acid molecule to a subject can be practiced either in vitro or in vivo. For practicing the method in vitro, cells can be obtained from a subject by standard methods and incubated (i.e., cultured) in vitro with a nucleic acid molecule and subsequently administered to the subject. Methods for isolating immune cells are known in the art. For further discussion of ex vivo genetic modification of cells followed by re-administration to a subject, see also U.S. Pat. No. 5,399,346 by W. F. Anderson et al.

In other embodiments, a nucleic acid molecule is administered to a subject in vivo, such as directly to an articulation site of a subject. For example, nucleic acids (e.g., recombinant expression vectors or antisense RNA) can be introduced into cells of a subject using methods known in the art for introducing nucleic acid (e.g., DNA) into cells in vivo. Examples of such methods include:

Direct Injection:

Naked DNA can be introduced into cells in vivo by directly injecting the DNA into the cells (see e.g., Acsadi et al. (1991) Nature 332:815-818; Wolff et al. (1990) Science 247:1465-1468). For example, a delivery apparatus (e.g., a “gene gun”) for injecting DNA into cells in vivo can be used. Such an apparatus is commercially available (e.g., from BioRad).

Cationic Lipids:

Naked DNA can be introduced into cells in vivo by complexing the DNA with cationic lipids or encapsulating the DNA in cationic liposomes. Examples of suitable cationic lipid formulations include N-[-1-(2,3-dioleoyloxy)propyl]N,N,N-triethylammonium chloride (DOTMA) and a 1:1 molar ratio of 1,2-dimyristyloxy-propyl-3-dimethylhydroxyethylammonium bromide (DMRIE) and dioleoyl phosphatidylethanolamine (DOPE) (see e.g., Logan, J. J. et al. (1995) Gene Therapy 2:38-49; San, H. et al. (1993) Human Gene Therapy 4:781-788).

Receptor-Mediated DNA Uptake:

Naked DNA can also be introduced into cells in vivo by complexing the DNA to a cation, such as polylysine, which is coupled to a ligand for a cell-surface receptor (see for example Wu, G. and Wu, C. H. (1988) J. Biol. Chem. 263:14621; Wilson et al. (1992) J. Biol. Chem. 267:963-967; and U.S. Pat. No. 5,166,320). Binding of the DNA-ligand complex to the receptor facilitates uptake of the DNA by receptor-mediated endocytosis. A DNA-ligand complex linked to adenovirus capsids which naturally disrupt endosomes, thereby releasing material into the cytoplasm can be used to avoid degradation of the complex by intracellular lysosomes (see for example Curiel et al. (1991) Proc. Natl. Acad. Sci. USA 88:8850; Cristiano et al. (1993) Proc. Natl. Acad. Sci. USA 90:2122-2126).

Retroviruses:

Defective retroviruses are well characterized for use in gene transfer for gene therapy purposes (for a review see Miller, A. D. (1990) Blood 76:271). A recombinant retrovirus can be constructed having a nucleotide sequences of interest incorporated into the retroviral genome. Additionally, portions of the retroviral genome can be removed to render the retrovirus replication defective. The replication defective retrovirus is then packaged into virions which can be used to infect a target cell through the use of a helper virus by standard techniques. Protocols for producing recombinant retroviruses and for infecting cells in vitro or in vivo with such viruses can be found in Current Protocols in Molecular Biology, Ausubel, F. M. et al. (eds.) Greene Publishing Associates, (1989), Sections 9.10-9.14 and other standard laboratory manuals. Examples of suitable retroviruses include pLJ, pZIP, pWE and pEM which are well known to those skilled in the art. Examples of suitable packaging virus lines include iv Crip, ψCre, ψ2 and ψAm. Retroviruses have been used to introduce a variety of genes into many different cell types, including epithelial cells, endothelial cells, lymphocytes, myoblasts, hepatocytes, bone marrow cells, in vitro and/or in vivo (see for example Eglitis, et al. (1985) Science 230:1395-1398; Danos and Mulligan (1988) Proc. Natl. Acad. Sci. USA 85:6460-6464; Wilson et al., (1988) Proc. Natl. Acad. Sci. USA 85:3014-3018; Armentano et al. (1990) Proc. Natl. Acad. Sci. USA 87:6141-6145; Huber et al., (1991) Proc. Natl. Acad. Sci. USA 88:8039-8043; Ferry et al., (1991) Proc. Natl. Acad. Sci. USA 88:8377-8381; Chowdhury et al. (1991) Science 254:1802-1805; van Beusechem et al. (1992) Proc. Natl. Acad. Sci. USA 89:7640-7644; Kay et al. (1992) Human Gene Therapy 3:641-647; Dai et al. (1992) Proc. Natl. Acad. Sci. USA 89:10892-10895; Hwu et al., (1993) J. Immunol. 150:4104-4115; U.S. Pat. No. 4,868,116; U.S. Pat. No. 4,980,286; PCT Application WO 89/07136; PCT Application WO 89/02468; PCT Application WO 89/05345; and PCT Application WO 92/07573). Retroviral vectors require target cell division in order for the retroviral genome (and foreign nucleic acid inserted into it) to be integrated into the host genome to stably introduce nucleic acid into the cell. Thus, it may be necessary to stimulate replication of the target cell.

Adenoviruses:

The genome of an adenovirus can be manipulated such that it encodes and expresses a gene product of interest but is inactivated in terms of its ability to replicate in a normal lytic viral life cycle. See for example Berkner et al. (1988) BioTechniques 6:616; Rosenfeld et al. (1991) Science 252:431-434; and Rosenfeld et al. (1992) Cell 68:143-155. Suitable adenoviral vectors derived from the adenovirus strain Ad type 5 d1324 or other strains of adenovirus (e.g., Ad2, Ad3, Ad7 etc.) are well known to those skilled in the art. Recombinant adenoviruses are advantageous in that they do not require dividing cells to be effective gene delivery vehicles and can be used to infect a wide variety of cell types, including airway epithelium (Rosenfeld et al. (1992) cited supra), endothelial cells (Lemarchand et al. (1992) Proc. Natl. Acad. Sci. USA 89:6482-6486), hepatocytes (Herz and Gerard (1993) Proc. Natl. Acad. Sci. USA 90:2812-2816) and muscle cells (Quantin et al. (1992) Proc. Natl. Acad. Sci. USA 89:2581-2584). Additionally, introduced adenoviral DNA (and foreign DNA contained therein) is not integrated into the genome of a host cell but remains episomal, thereby avoiding potential problems that can occur as a result of insertional mutagenesis in situations where introduced DNA becomes integrated into the host genome (e.g., retroviral DNA). Moreover, the carrying capacity of the adenoviral genome for foreign DNA is large (up to 8 kilobases) relative to other gene delivery vectors (Berkner et al. cited supra; Haj-Ahmand and Graham (1986) J. Virol. 57:267). Most replication-defective adenoviral vectors currently in use are deleted for all or parts of the viral E1 and E3 genes but retain as much as 80% of the adenoviral genetic material.

Adeno-Associated Viruses:

Adeno-associated virus (AAV) is a naturally occurring defective virus that requires another virus, such as an adenovirus or a herpes virus, as a helper virus for efficient replication and a productive life cycle. (For a review see Muzyczka et al. Curr. Topics in Micro. and Immunol. (1992) 158:97-129). It is also one of the few viruses that may integrate its DNA into non-dividing cells, and exhibits a high frequency of stable integration (see for example Flotte et al., (1992) Am. J. Respir. Cell. Mol. Biol. 7:349-356; Samulski et al., (1989) J. Virol. 63:3822-3828; and McLaughlin et al. (1989) J. Virol. 62:1963-1973). Vectors containing as little as 300 base pairs of AAV can be packaged and can integrate. Space for exogenous DNA is limited to about 4.5 kb. An AAV vector such as that described in Tratschin et al. (1985) Mol. Cell. Biol. 5:3251-3260 can be used to introduce DNA into cells. A variety of nucleic acids have been introduced into different cell types using AAV vectors (see for example Hermonat et al. (1984) Proc. Natl. Acad. Sci. USA 131:6466-6470; Tratschin et al. (1985) Mol. Cell. Biol. 4:2072-2081; Wondisford et al. (1988) Mol. Endocrinol. 2:32-39; Tratschin et al. (1984) J. Virol. 51:611-619; and Flotte et al. (1993) J. Biol. Chem. 268:3781-3790).

The efficacy of a particular expression vector system and method of introducing nucleic acid into a cell can be assessed by standard approaches routinely used in the art. For example, DNA introduced into a cell can be detected by a filter hybridization technique (e.g., Southern blotting) and RNA produced by transcription of introduced DNA can be detected, for example, by Northern blotting, RNase protection or reverse transcriptase-polymerase chain reaction (RT-PCR). The gene product can be detected by an appropriate assay, for example by immunological detection of a produced protein, such as with a specific antibody, or by a functional assay to detect a functional activity of the gene product, such as an enzymatic assay.

The invention also provides methods (also referred to herein as “screening assays”) for identifying LT-β-R and/or NFκB1 activators and TRAF3 inhibitors, i.e., candidate or test compounds or agents (e.g., proteins, peptides, peptidomimetics, peptoids, small molecules or other drugs), which are suitable for use in the combination therapies of the invention to treat cancer by activating the expression and/or activity LT-β-R and/or NFκB1 and/or inhibiting the expression and/or activity TRAF3. Such assays typically comprise a reaction between LT-β-R, NFκB1, or TRAF3 and one or more assay components. The other components may be either the test compound itself, or a combination of test compounds and a natural binding partner of LT-β-R, NFκB1, or TRAF3. Compounds identified via assays such as those described herein may be useful, for example, for modulating, e.g., inhibiting, ameliorating, treating, or preventing cancer.

The test compounds used in the screening assays of the present invention may be obtained from any available source, including systematic libraries of natural and/or synthetic compounds. Test compounds may also be obtained by any of the numerous approaches in combinatorial library methods known in the art, including: biological libraries; peptoid libraries (libraries of molecules having the functionalities of peptides, but with a novel, non-peptide backbone which are resistant to enzymatic degradation but which nevertheless remain bioactive; see, e.g., Zuckermann et al., 1994, J. Med. Chem. 37:26′78-85); spatially addressable parallel solid phase or solution phase libraries; synthetic library methods requiring deconvolution; the ‘one-bead one-compound’ library method; and synthetic library methods using affinity chromatography selection. The biological library and peptoid library approaches are limited to peptide libraries, while the other four approaches are applicable to peptide, non-peptide oligomer or small molecule libraries of compounds (Lam, 1997, Anticancer Drug Des. 12:145).

Examples of methods for the synthesis of molecular libraries can be found in the art, for example in: DeWitt et al. (1993) Proc. Natl. Acad. Sci. U.S.A. 90:6909; Erb et al. (1994) Proc. Natl. Acad. Sci. USA 91:11422; Zuckermann et al. (1994). J. Med. Chem. 37:2678; Cho et al. (1993) Science 261:1303; Carrell et al. (1994) Angew. Chem. Int. Ed. Engl. 33:2059; Carell et al. (1994) Angew. Chem. Int. Ed Engl. 33:2061; and in Gallop et al. (1994) J. Med. Chem. 37:1233.

Libraries of compounds may be presented in solution (e.g., Houghten, 1992, Biotechniques 13:412-421), or on beads (Lam, 1991, Nature 354:82-84), chips (Fodor, 1993, Nature 364:555-556), bacteria and/or spores, (Ladner, U.S. Pat. No. 5,223,409), plasmids (Cull et al, 1992, Proc Natl Acad Sci USA 89:1865-1869) or on phage (Scott and Smith, 1990, Science 249:386-390; Devlin, 1990, Science 249:404-406; Cwirla el al, 1990, Proc. Natl. Acad. Sci. 87:6378-6382; Felici, 1991, J. Mol. Biol. 222:301-310; Ladner, supra.).

The screening methods of the invention generally comprise contacting a cancer cell, e.g., an adenocarcinoma cancer cell, with a test compound and determining the ability of the test compound to modulate the expression and/or activity of LT-β-R, NFκB1, TRAF2, and/or TRAF3, and/or the TRAF3/TRAF2 ratio in the cell. The expression and/or activity of LT-β-R, NFκB1, TRAF2, and/or TRAF3, and/or the TRAF3/TRAF2 ratio can be determined as described herein.

In one embodiment, the ability of a compound to modulate the activity of LT-β-R, NFκB1, or TRAF3 is measured by determining apoptotic cell death.

The ability of a compound to modulate apoptosis can be determined by, for example, detecting cytochrome C release from mitochondria during cell apoptosis, e.g., plasma cell apoptosis (as described in, for example, Bossy-Wetzel E. et al. (2000) Methods in Enzymol. 322:235-42). Other exemplary assays include: cytofluorometric quantitation of nuclear apoptosis induced in a cell-free system (as described in, for example, Lorenzo H. K. et al. (2000) Methods in Enzymol. 322:198-201); apoptotic nuclease assays (as described in, for example, Hughes F. M. (2000) Methods in Enzymol. 322:47-62); analysis of apoptotic cells, e.g., apoptotic plasma cells, by flow and laser scanning cytometry (as described in, for example, Darzynkiewicz Z. et al. (2000) Methods in Enzymol. 322:18-39); detection of apoptosis by annexin V labeling (as described in, for example, Bossy-Wetzel E. et al. (2000) Methods in Enzymol. 322:15-18); transient transfection assays for cell death genes (as described in, for example, Miura M. et al. (2000) Methods in Enzymol. 322:480-92); and assays that detect DNA cleavage in apoptotic cells, e.g., apoptotic plasma cells (as described in, for example, Kauffman S. H. et al. (2000) Methods in Enzymol 322:3-15). Apoptosis can also be measured by propidium iodide staining or by TUNEL assay.

In another embodiment, the ability of a compound to modulate the activity of LT-β-R, NFκB1, or TRAF3 is measured by determining LT-β-R and/or NFκB1 signaling, e.g., by measuring NFκB1-dependent gene activation, and/or the TRAF3/TRAF2 ratio.

In one embodiment, the ability of a compound to modulate the activity of TRAF3 is measured by determining the phosphorylation state of IκBα and RelA.

In yet another embodiment, the invention provides assays for screening candidate or test compounds which bind to LT-β-R, NFκB1, or TRAF3 or biologically active portions thereof. Determining the ability of the test compound to directly bind to a marker can be accomplished, for example, by coupling the compound with a radioisotope or enzymatic label such that binding of the compound to the marker can be determined by detecting the labeled marker compound in a complex. For example, compounds (e.g., marker substrates) can be labeled with ¹³¹I, ¹²⁵I, ³⁵S, ¹⁴C, or ³H, either directly or indirectly, and the radioisotope detected by direct counting of radioemission or by scintillation counting. Alternatively, assay components can be enzymatically labeled with, for example, horseradish peroxidase, alkaline phosphatase, or luciferase, and the enzymatic label detected by determination of conversion of an appropriate substrate to product.

This invention further pertains to novel agents identified by the above-described screening assays. Accordingly, it is within the scope of this invention to further use an agent identified as described herein in an appropriate animal model. For example, an agent capable of modulating the expression and/or activity of LT-β-R, NFκB1, and/or TRAF3 identified as described herein can be used in an animal model to determine the efficacy, toxicity, or side effects of treatment with such an agent. Alternatively, an agent identified as described herein can be used in an animal model to determine the mechanism of action of such an agent. Furthermore, this invention pertains to uses of novel agents identified by the above-described screening assays for treatment as described above.

### 3. Methods for Obtaining Samples and Determining the Amount of TRAF3, TRAF2, and/or p53 and/or the TRAF3/TRAF2 Ratio

Samples useful in the methods of the invention include any tissue, cell, biopsy, or bodily fluid sample that expresses TRAF3, TRAF2, and/or p53. In one embodiment, a sample is a tumor sample, e.g., a colon tumor or a cervical tumor sample.

Body samples may be obtained from a subject by a variety of techniques known in the art including, for example, by the use of a biopsy or by scraping or swabbing an area or by using a needle to aspirate. Methods for collecting various body samples are well known in the art.

Tissue samples suitable for detecting and quantitating TRAF3, TRAF2, and/or p53 may be fresh, frozen, or fixed according to methods known to one of skill in the art. In one embodiment, suitable tissue samples are sectioned and placed on a microscope slide for further analyses. In another embodiment, suitable solid samples, i.e., tissue samples, are solubilized and/or homogenized and subsequently analyzed as soluble extracts.

Once the sample is obtained any method known in the art to be suitable for detecting and quantitating a marker suitable for use in the methods of the invention, i.e., TRAF3, TRAF2, and/or p53, may be used (either at the nucleic acid or, preferably, at the protein level). Such methods are well known in the art and include but are not limited to Western blots, Northern blots, Southern blots, immunohistochemistry, ELISA, e.g., amplified ELISA, immunoprecipitation, immunofluorescence, flow cytometry, immunocytochemistry, mass spectrometrometric analyses, e.g., MALDI-TOF and SELDI-TOF, nucleic acid hybridization techniques, nucleic acid reverse transcription methods, and nucleic acid amplification methods.

In one embodiment of the invention, the amount of TRAF3, TRAF2, and/or p53 and/or the TRAF3/TRAF2 ratio, is determined by detecting or quantifying the expressed polypeptide. The polypeptide can be detected and quantified by any of a number of means well known to those of skill in the art. These may include analytic biochemical methods such as electrophoresis, capillary electrophoresis, high performance liquid chromatography (HPLC), thin layer chromatography (TLC), hyperdiffusion chromatography, and the like, or various immunological methods such as fluid or gel precipitin reactions, immunodiffusion (single or double), immunoelectrophoresis, radioimmunoassay (RIA), enzyme-linked immunosorbent assays (ELISAs), immunofluorescent assays, Western blotting, and the like.

Proteins from cells can be isolated using techniques that are well known to those of skill in the art. The protein isolation methods employed can, for example, be such as those described in Harlow and Lane (Harlow and Lane, 1988, Antibodies: A Laboratory Manual, Cold Spring Harbor Laboratory Press, Cold Spring Harbor, N.Y.).

In one embodiment, the agent for detecting a TRAF3, TRAF2, and/or p53 polypeptide is an antibody capable of binding to a TRAF3, TRAF2, and/or p53 polypeptide. Antibodies can be polyclonal, or more preferably, monoclonal. An intact antibody, or a fragment thereof (e.g., Fab or F(ab′)₂) can be used. In one embodiment, an antibody for detecting and quantitating TRAF3 is the H-20 antibody. In another embodiment, an antibody for detecting and quantitating TRAF3 is the H-122 antibody.

In one embodiment, an antibody for detecting and quantitating TRAF2 is the H-249 antibody. In one embodiment, an antibody for detecting and quantitating p53 is DO7 (Dako Cytomation, #M 7001).

In one embodiment, the antibody is labeled with a detectable label. The term “labeled”, with regard to the probe or antibody, is intended to encompass direct labeling of the probe or antibody by coupling (i.e., physically linking) a detectable substance to the probe or antibody, as well as indirect labeling of the probe or antibody by reactivity with another reagent that is directly labeled. Examples of indirect labeling include detection of a primary antibody using a fluorescently labeled secondary antibody and end-labeling of a DNA probe with biotin such that it can be detected with fluorescently labeled streptavidin.

In a one embodiment, the antibody is labeled, e.g. a radio-labeled, chromophore-labeled, fluorophore-labeled, or enzyme-labeled antibody. In another embodiment, an antibody derivative (e.g. an antibody conjugated with a substrate or with the protein or ligand of a protein-ligand pair {e.g. biotin-streptavidin}), or an antibody fragment (e.g. a single-chain antibody, an isolated antibody hypervariable domain, etc.) which binds specifically with a TRAF3, TRAF2, and/or p53 protein, such as the protein encoded by the open reading frame corresponding to TRAF3, TRAF2, and/or p53 or such a protein which has undergone all or a portion of its normal post-translational modification, is used.

A skilled artisan can readily adapt known protein/antibody detection methods for use in determining the amount of a marker of the present invention (i.e., TRAF3, TRAF2, and/or p53, and/or TRAF3/TRAF2 ratio).

In one format, antibodies, or antibody fragments, can be used in methods such as Western blots or immunofluorescence techniques to detect the expressed proteins. In such uses, it is generally preferable to immobilize either the antibody or proteins on a solid support. Suitable solid phase supports or carriers include any support capable of binding an antigen or an antibody. Well-known supports or carriers include glass, polystyrene, polypropylene, polyethylene, dextran, nylon, amylases, natural and modified celluloses, polyacrylamides, gabbros, and magnetite.

One skilled in the art will know many other suitable carriers for binding antibody or antigen, and will be able to adapt such support for use with the present invention. For example, protein isolated from cells can be run on a polyacrylamide gel electrophoresis and immobilized onto a solid phase support such as nitrocellulose. The support can then be washed with suitable buffers followed by treatment with the detectably labeled antibody. The solid phase support can then be washed with the buffer a second time to remove unbound antibody. The amount of bound label on the solid support can then be detected by conventional means. Means of detecting proteins using electrophoretic techniques are well known to those of skill in the art (see generally, R. Scopes (1982) Protein Purification, Springer-Verlag, N.Y.; Deutscher, (1990) Methods in Enzymology Vol. 182: Guide to Protein Purification, Academic Press, Inc., N.Y.).

In one embodiment, Western blot (immunoblot) analysis is used to detect and quantify the presence of a polypeptide in the sample. This technique generally comprises separating sample proteins by gel electrophoresis on the basis of molecular weight, transferring the separated proteins to a suitable solid support, (such as a nitrocellulose filter, a nylon filter, or derivatized nylon filter), and incubating the sample with the antibodies that specifically bind a polypeptide. The anti-polypeptide antibodies specifically bind to the polypeptide on the solid support. These antibodies may be directly labeled or alternatively may be subsequently detected using labeled antibodies (e.g., labeled sheep anti-human antibodies) that specifically bind to the anti-polypeptide.

In one embodiment of the invention, the amount of TRAF3, TRAF2, and/or p53 and/or the TRAF3/TRAF2 is determined by Western blot analysis and subsequent densitometric analysis.

In another embodiment, the polypeptide is detected using an immunoassay. As used herein, an immunoassay is an assay that utilizes an antibody to specifically bind to the analyte. The immunoassay is thus characterized by detection of specific binding of a polypeptide to an anti-antibody as opposed to the use of other physical or chemical properties to isolate, target, and quantify the analyte.

The polypeptide is detected and/or quantified using any of a number of well recognized immunological binding assays (see, e.g., U.S. Pat. Nos. 4,366,241; 4,376,110; 4,517,288; and 4,837,168). For a review of the general immunoassays, see also Asai (1993) Methods in Cell Biology Volume 37: Antibodies in Cell Biology, Academic Press, Inc. New York; Stites & Terr (1991) Basic and Clinical Immunology 7th Edition.

Immunological binding assays (or immunoassays) typically utilize a “capture agent” to specifically bind to and often immobilize the analyte (polypeptide or subsequence). The capture agent is a moiety that specifically binds to the analyte. In a preferred embodiment, the capture agent is an antibody that specifically binds a polypeptide. The antibody (anti-peptide) may be produced by any of a number of means well known to those of skill in the art.

Immunoassays also often utilize a labeling agent to specifically bind to and label the binding complex formed by the capture agent and the analyte. The labeling agent may itself be one of the moieties comprising the antibody/analyte complex. Thus, the labeling agent may be a labeled polypeptide or a labeled anti-antibody. Alternatively, the labeling agent may be a third moiety, such as another antibody, that specifically binds to the antibody/polypeptide complex.

In one embodiment, the labeling agent is a second human antibody bearing a label. Alternatively, the second antibody may lack a label, but it may, in turn, be bound by a labeled third antibody specific to antibodies of the species from which the second antibody is derived. The second can be modified with a detectable moiety, e.g. as biotin, to which a third labeled molecule can specifically bind, such as enzyme-labeled streptavidin.

Other proteins capable of specifically binding immunoglobulin constant regions, such as protein A or protein G may also be used as the label agent. These proteins are normal constituents of the cell walls of streptococcal bacteria. They exhibit a strong non-immunogenic reactivity with immunoglobulin constant regions from a variety of species (see, generally Kronval, et al. (1973) J. Immunol., 111: 1401-1406, and Akerstrom (1985) J. Immunol., 135: 2589-2542).

As indicated above, immunoassays for the detection and/or quantification of a polypeptide can take a wide variety of formats well known to those of skill in the art.

Preferred immunoassays for detecting a polypeptide are either competitive or noncompetitive. Noncompetitive immunoassays are assays in which the amount of captured analyte is directly measured. In one preferred “sandwich” assay, for example, the capture agent (anti-peptide antibodies) can be bound directly to a solid substrate where they are immobilized. These immobilized antibodies then capture polypeptide present in the test sample. The polypeptide thus immobilized is then bound by a labeling agent, such as a second human antibody bearing a label.

In competitive assays, the amount of analyte (polypeptide) present in the sample is measured indirectly by measuring the amount of an added (exogenous) analyte (polypeptide) displaced (or competed away) from a capture agent (anti-peptide antibody) by the analyte present in the sample. In one competitive assay, a known amount of, in this case, a polypeptide is added to the sample and the sample is then contacted with a capture agent. The amount of polypeptide bound to the antibody is inversely proportional to the concentration of polypeptide present in the sample.

In one embodiment, the antibody is immobilized on a solid substrate. The amount of polypeptide bound to the antibody may be determined either by measuring the amount of polypeptide present in a polypeptide/antibody complex, or alternatively by measuring the amount of remaining uncomplexed polypeptide. The amount of polypeptide may be detected by providing a labeled polypeptide.

In vivo techniques for determining the amount of TRAF3, TRAF2, and/or p53 protein, and/or the TRAF3/TRAF2 ratio, may also be used in the methods of the invention and include introducing into a subject a labeled antibody directed against the protein. For example, the antibody can be labeled with a radioactive marker whose presence and location in a subject can be detected by standard imaging techniques.

In one embodiment of the invention, proteomic methods, e.g., mass spectrometry, are used for detecting and quantitating TRAF3, TRAF2, and/or p53, and/or the TRAF3/TRAF2 ratio. For example, matrix-associated laser desorption/ionization time-of-flight mass spectrometry (MALDI-TOF MS) or surface-enhanced laser desorption/ionization time-of-flight mass spectrometry (SELDI-TOF MS) which involves the application of a biological sample, such as serum, to a protein-binding chip (Wright, G. L., Jr., et al. (2002) Expert Rev Mol Diagn 2:549; Li, J., et al. (2002) Clin Chem 48:1296; Laronga, C., et al. (2003) Dis Markers 19:229; Petricoin, E. F., et al. (2002) 359:572; Adam, B. L., et al. (2002) Cancer Res 62:3609; Tolson, J., et al. (2004) Lab Invest 84:845; Xiao, Z., et al. (2001) Cancer Res 61:6029) can be used to detect and quantitate TRAF3, TRAF2, and/or p53 proteins. Mass spectrometric methods are described in, for example, U.S. Pat. Nos. 5,622,824, 5,605,798 and 5,547,835, the entire contents of each of which are incorporated herein by reference.

In another embodiment of the invention, the amount of TRAF3, TRAF2, and/or p53, and/or the TRAF3/TRAF2 ratio is determined by measuring and quantitating the amount of TRAF3, TRAF2, and/or p53 nucleic acid. In yet other embodiments, the presence or absence of p53 is determined at the nucleic acid level. Nucleic acid-based techniques for assessing expression are well known in the art and include, for example, determining the amount of TRAF3, TRAF2, and/or p53 mRNA in a body sample. Many expression detection methods use isolated RNA. Any RNA isolation technique that does not select against the isolation of mRNA can be utilized for the purification of RNA from cells that express TRAF3, TRAF2, and/or p53 (see, e.g., Ausubel et al., ed., (1987-1999) Current Protocols in Molecular Biology (John Wiley & Sons, New York). Additionally, large numbers of tissue samples can readily be processed using techniques well known to those of skill in the art, such as, for example, the single-step RNA isolation process of Chomczynski (1989, U.S. Pat. No. 4,843,155).

Methods of detecting and/or quantifying the gene transcript (mRNA or cDNA made there from) using nucleic acid hybridization techniques are known to those of skill in the art (see Sambrook et al. supra). For example, one method for evaluating the presence, absence, or amount of cDNA involves a Southern transfer as described above. Briefly, the mRNA is isolated (e.g. using an acid guanidinium-phenol-chloroform extraction method, Sambrook et al. supra.) and reverse transcribed to produce cDNA. The cDNA is then optionally digested and run on a gel in buffer and transferred to membranes. Hybridization is then carried out using the nucleic acid probes specific for the target cDNA.

A general principle of such assays involves preparing a sample or reaction mixture that may contain a marker, i.e., TRAF3, TRAF2, and/or p53, and a probe, under appropriate conditions and for a time sufficient to allow the marker and probe to interact and bind, thus forming a complex that can be removed and/or detected in the reaction mixture. These assays can be conducted in a variety of ways.

For example, one method to conduct such an assay would involve anchoring the marker or probe onto a solid phase support, also referred to as a substrate, and detecting target marker/probe complexes anchored on the solid phase at the end of the reaction. In one embodiment of such a method, a sample from a subject, which is to be assayed for presence and/or amount of marker, can be anchored onto a carrier or solid phase support. In another embodiment, the reverse situation is possible, in which the probe can be anchored to a solid phase and a sample from a subject can be allowed to react as an unanchored component of the assay.

There are many established methods for anchoring assay components to a solid phase. These include, without limitation, marker or probe molecules which are immobilized through conjugation of biotin and streptavidin. Such biotinylated assay components can be prepared from biotin-NHS (N-hydroxy-succinimide) using techniques known in the art (e.g., biotinylation kit, Pierce Chemicals, Rockford, Ill.), and immobilized in the wells of streptavidin-coated 96 well plates (Pierce Chemical). In certain embodiments, the surfaces with immobilized assay components can be prepared in advance and stored.

Other suitable carriers or solid phase supports for such assays include any material capable of binding the class of molecule to which the marker or probe belongs. Well-known supports or carriers include, but are not limited to, glass, polystyrene, nylon, polypropylene, polyethylene, dextran, amylases, natural and modified celluloses, polyacrylamides, gabbros, and magnetite.

In order to conduct assays with the above-mentioned approaches, the non-immobilized component is added to the solid phase upon which the second component is anchored. After the reaction is complete, uncomplexed components may be removed (e.g., by washing) under conditions such that any complexes formed will remain immobilized upon the solid phase. The detection of marker/probe complexes anchored to the solid phase can be accomplished in a number of methods outlined herein.

In a preferred embodiment, the probe, when it is the unanchored assay component, can be labeled for the purpose of detection and readout of the assay, either directly or indirectly, with detectable labels discussed herein and which are well-known to one skilled in the art.

It is also possible to directly detect marker/probe complex formation without further manipulation or labeling of either component (marker or probe), for example by utilizing the technique of fluorescence energy transfer (see, for example, Lakowicz et al., U.S. Pat. No. 5,631,169; Stavrianopoulos, et al., U.S. Pat. No. 4,868,103). A fluorophore label on the first, ‘donor’ molecule is selected such that, upon excitation with incident light of appropriate wavelength, its emitted fluorescent energy will be absorbed by a fluorescent label on a second ‘acceptor’ molecule, which in turn is able to fluoresce due to the absorbed energy. Alternately, the ‘donor’ protein molecule may simply utilize the natural fluorescent energy of tryptophan residues. Labels are chosen that emit different wavelengths of light, such that the ‘acceptor’ molecule label may be differentiated from that of the ‘donor’. Since the efficiency of energy transfer between the labels is related to the distance separating the molecules, spatial relationships between the molecules can be assessed. In a situation in which binding occurs between the molecules, the fluorescent emission of the ‘acceptor’ molecule label in the assay should be maximal. An FET binding event can be conveniently measured through standard fluorometric detection means well known in the art (e.g., using a fluorimeter).

In another embodiment, determination of the ability of a probe to recognize a marker can be accomplished without labeling either assay component (probe or marker) by utilizing a technology such as real-time Biomolecular Interaction Analysis (BIA) (see, e.g., Sjolander, S. and Urbaniczky, C., 1991, Anal. Chem. 63:2338-2345 and Szabo et al., 1995, Curr. Opin. Struct. Biol. 5:699-705). As used herein, “BIA” or “surface plasmon resonance” is a technology for studying biospecific interactions in real time, without labeling any of the interactants (e.g., BIAcore). Changes in the mass at the binding surface (indicative of a binding event) result in alterations of the refractive index of light near the surface (the optical phenomenon of surface plasmon resonance (SPR)), resulting in a detectable signal which can be used as an indication of real-time reactions between biological molecules.

Alternatively, in another embodiment, analogous assays can be conducted with marker and probe as solutes in a liquid phase. In such an assay, the complexed marker and probe are separated from uncomplexed components by any of a number of standard techniques, including but not limited to: differential centrifugation, chromatography, electrophoresis and immunoprecipitation. In differential centrifugation, marker/probe complexes may be separated from uncomplexed assay components through a series of centrifugal steps, due to the different sedimentation equilibria of complexes based on their different sizes and densities (see, for example, Rivas, G., and Minton, A. P., 1993, Trends Biochem Sci. 18(8):284-7). Standard chromatographic techniques may also be utilized to separate complexed molecules from uncomplexed ones. For example, gel filtration chromatography separates molecules based on size, and through the utilization of an appropriate gel filtration resin in a column format, for example, the relatively larger complex may be separated from the relatively smaller uncomplexed components. Similarly, the relatively different charge properties of the marker/probe complex as compared to the uncomplexed components may be exploited to differentiate the complex from uncomplexed components, for example, through the utilization of ion-exchange chromatography resins. Such resins and chromatographic techniques are well known to one skilled in the art (see, e.g., Heegaard, N. H, 1998, J. Mol. Recognit. Winter 11(1-6):141-8; Hage, D. S., and Tweed, S. A. J Chromatogr B Biomed Sci Appl 1997 Oct. 10; 699(1-2):499-525). Gel electrophoresis may also be employed to separate complexed assay components from unbound components (see, e.g., Ausubel et al., ed., Current Protocols in Molecular Biology, John Wiley & Sons, New York, 1987-1999). In this technique, protein or nucleic acid complexes are separated based on size or charge, for example. In order to maintain the binding interaction during the electrophoretic process, non-denaturing gel matrix materials and conditions in the absence of reducing agent are typically preferred. Appropriate conditions to the particular assay and components thereof will be well known to one skilled in the art.

In a particular embodiment, the amount of mRNA corresponding to the marker can be determined both by in situ and by in vitro formats in a biological sample using methods known in the art. Many expression detection methods use isolated RNA. For in vitro methods, any RNA isolation technique that does not select against the isolation of mRNA can be utilized for the purification of RNA from cells (see, e.g., Ausubel et al., ed., Current Protocols in Molecular Biology, John Wiley & Sons, New York 1987-1999). Additionally, large numbers of tissue samples can readily be processed using techniques well known to those of skill in the art, such as, for example, the single-step RNA isolation process of Chomczynski (1989, U.S. Pat. No. 4,843,155).

The isolated nucleic acid can be used in hybridization or amplification assays that include, but are not limited to, Southern or Northern analyses, polymerase chain reaction analyses and probe arrays. One preferred diagnostic method for the detection of the amount of mRNA involves contacting the isolated mRNA with a nucleic acid molecule (probe) that can hybridize to the mRNA encoded by the gene being detected. The nucleic acid probe can be, for example, a full-length cDNA, or a portion thereof such as an oligonucleotide of at least 7, 15, 30, 50, 100, 250 or 500 nucleotides in length and sufficient to specifically hybridize under stringent conditions to a mRNA or genomic DNA encoding a marker of the present invention. Other suitable probes for use in the diagnostic assays of the invention are described herein. Hybridization of an mRNA with the probe indicates that the marker in question is being expressed.

In one format, the mRNA is immobilized on a solid surface and contacted with a probe, for example by running the isolated mRNA on an agarose gel and transferring the mRNA from the gel to a membrane, such as nitrocellulose. In an alternative format, the probe(s) are immobilized on a solid surface and the mRNA is contacted with the probe(s), for example, in an Affymetrix gene chip array. A skilled artisan can readily adapt known mRNA detection methods for use in detecting the amount of mRNA encoded by the markers of the present invention.

The probes can be full length or less than the full length of the nucleic acid sequence encoding the protein. Shorter probes are empirically tested for specificity. Preferably nucleic acid probes are 20 bases or longer in length. (See, e.g., Sambrook et al. for methods of selecting nucleic acid probe sequences for use in nucleic acid hybridization.) Visualization of the hybridized portions allows the qualitative determination of the presence or absence of cDNA.

An alternative method for determining the amount of a transcript corresponding to a marker of the present invention in a sample involves the process of nucleic acid amplification, e.g., by RT-PCR (the experimental embodiment set forth in Mullis, 1987, U.S. Pat. No. 4,683,202), ligase chain reaction (Barany, 1991, Proc. Natl. Acad. Sci. USA, 88:189-193), self sustained sequence replication (Guatelli et al., 1990, Proc. Natl. Acad. Sci. USA 87:1874-1878), transcriptional amplification system (Kwoh et al., 1989, Proc. Natl. Acad. Sci. USA 86:1173-1177), Q-Beta Replicase (Lizardi et al., 1988, Bio/Technology 6:1197), rolling circle replication (Lizardi et al., U.S. Pat. No. 5,854,033) or any other nucleic acid amplification method, followed by the detection of the amplified molecules using techniques well known to those of skill in the art. These detection schemes are especially useful for the detection of nucleic acid molecules if such molecules are present in very low numbers. In particular aspects of the invention, TRAF3, TRAF2, and/or p53 expression is assessed by quantitative fluorogenic RT-PCR (i.e., the TaqMan™ System). Such methods typically utilize pairs of oligonucleotide primers that are specific for the marker. Methods for designing oligonucleotide primers specific for a known sequence are well known in the art.

Fluorogenic rtPCR may also be used in the methods of the invention. In fluorogenic rtPCR, quantitation is based on amount of fluorescence signals, e.g., TaqMan and sybr green. These detection schemes are especially useful for the detection of nucleic acid molecules if such molecules are present in very low numbers. As used herein, amplification primers are defined as being a pair of nucleic acid molecules that can anneal to 5′ or 3′ regions of a gene (plus and minus strands, respectively, or vice-versa) and contain a short region in between. In general, amplification primers are from about 10 to 30 nucleotides in length and flank a region from about 50 to 200 nucleotides in length. Under appropriate conditions and with appropriate reagents, such primers permit the amplification of a nucleic acid molecule comprising the nucleotide sequence flanked by the primers.

For in situ methods, mRNA does not need to be isolated from the cells prior to detection. In such methods, a cell or tissue sample is prepared/processed using known histological methods. The sample is then immobilized on a support, typically a glass slide, and then contacted with a probe that can hybridize to mRNA that encodes the marker.

In another embodiment, amount of a marker is assessed by preparing genomic DNA or mRNA/cDNA (i.e. a transcribed polynucleotide) from cells in a subject sample, and by hybridizing the genomic DNA or mRNA/cDNA with a reference polynucleotide which is a complement of a polynucleotide comprising the marker, and fragments thereof. cDNA can, optionally, be amplified using any of a variety of polymerase chain reaction methods prior to hybridization with the reference polynucleotide. Expression amounts of one or more markers can likewise be detected using quantitative PCR (QPCR) to assess the amount of expression of the marker(s). Alternatively, any of the many known methods of detecting mutations or variants (e.g. single nucleotide polymorphisms, deletions, etc.) of a marker of the invention may be used to detect occurrence of a mutated marker in a subject.

In a related embodiment, a mixture of transcribed polynucleotides obtained from the sample is contacted with a substrate having fixed thereto a polynucleotide complementary to or homologous with at least a portion (e.g. at least 7, 10, 15, 20, 25, 30, 40, 50, 100, 500, or more nucleotide residues) of a marker of the invention. If polynucleotides complementary to or homologous with are differentially detectable on the substrate (e.g. detectable using different chromophores or fluorophores, or fixed to different selected positions), then the amounts of expression of a plurality of markers can be assessed simultaneously using a single substrate (e.g. a “gene chip” microarray of polynucleotides fixed at selected positions). When a method of assessing marker expression is used which involves hybridization of one nucleic acid with another, it is preferred that the hybridization be performed under stringent hybridization conditions.

In one embodiment of the invention, microarrays are used to detect TRAF3, TRAF2, and/or p53 expression. Microarrays are particularly well suited for this purpose because of the reproducibility between different experiments. DNA microarrays provide one method for the simultaneous measurement of the expression amounts of large numbers of genes. Each array consists of a reproducible pattern of capture probes attached to a solid support. Labeled RNA or DNA is hybridized to complementary probes on the array and then detected by laser scanning. Hybridization intensities for each probe on the array are determined and converted to a quantitative value representing relative gene expression levels (amounts). See, U.S. Pat. Nos. 6,040,138, 5,800,992 and 6,020,135, 6,033,860, and 6,344,316, which are incorporated herein by reference. High-density oligonucleotide arrays are particularly useful for determining the gene expression profile for a large number of RNA's in a sample.

In another embodiment, a combination of methods to assess the expression of a marker is utilized.

The assays of this invention are scored (as positive or negative or amount of polypeptide and/or mRNA) according to standard methods well known to those of skill in the art. The particular method of scoring will depend on the assay format and choice of label. For example, a Western Blot assay can be scored by visualizing the colored product produced by the enzymatic label. A clearly visible colored band or spot at the correct molecular weight is scored as a positive result, while the absence of a clearly visible spot or band is scored as a negative. The intensity of the band or spot can provide a quantitative measure of polypeptide.

As an alternative to making determinations of the amount of TRAF3, TRAF2, and/or p53, and/or the TRAF3/TRAF2 based on the absolute expression level of the marker, determinations may be based on the normalized expression level of the marker. Expression levels are normalized by correcting the absolute expression level of a marker by comparing its expression to the expression of a protein that is not a marker, e.g.; a housekeeping gene that is constitutively expressed. Suitable genes for normalization include housekeeping genes such as the actin gene, or epithelial cell-specific genes. This normalization allows the comparison of the expression level in one sample, e.g., a subject sample, to another sample, e.g., a non-cancerous sample, a sample from a tumor that has known resistance to an LT-βR activating agent, and/or a sample from a tumor that has known sensitivity to an LT-βR activating agent, or between samples from different sources.

Alternatively, the expression level can be provided as a relative expression level. To determine a relative expression level of a marker, the level of expression of the marker is determined for 10 or more samples of normal versus cancer cell isolates, preferably 50 or more samples, prior to the determination of the expression level for the sample in question. The mean expression level of each of the proteins assayed in the larger number of samples is determined and this is used as a baseline expression level for the marker. The expression level of the marker determined for the test sample (absolute level of expression) is then divided by the mean expression value obtained for that marker. This provides a relative expression level.

Preferably, the samples used in the baseline determination will be from cancer cells or normal cells of the same tissue type. The choice of the cell source is dependent on the use of the relative expression level. Using expression found in normal tissues as a mean expression score aids in validating whether the marker assayed is specific to the tissue from which the cell was derived (versus normal cells). In addition, as more data is accumulated, the mean expression value can be revised, providing improved relative expression values based on accumulated data. Expression data from normal cells provides a means for grading the severity of the cancer state.

Typically denisotmetric analysis is use to determine normalized and/or relative expression levels (amounts) and is a technique known to one of skill in the art.

### 4. Agents for Use in the Methods of the Invention

According to the methods of the invention, a tumor or subject is treated by administration of a combination therapy of the invention. In certain aspects of the invention, a tumor or subject is treated by administration of an LT-β-R activating agent in combination with an NFκB1 activating agent or an agent that inhibits TRAF3 activity. Examples of suitable activating agents include active LT-β-R or NFκB1 polypeptides and nucleic acid molecules encoding LT-β-R or NFκB1 that are introduced into the cell to increase LT-β-R or NFκB1 expression and/or activity in the cell, and agonistic binding molecules. Examples of inhibitory agents of TRAF3 include antisense nucleic acid molecules, small molecules, antagonistic binding molecules, and are described in further detail below.

### A. Activating Agents

In one embodiment, an activating agent is a nucleic acid molecule encoding a polypeptide, i.e., a LT-β-R or NFκB1 polypeptide, wherein the nucleic acid molecule is introduced into the cell in a form suitable for expression of the active polypeptide in the cell. To express a LT-β-R or NFκB1 polypeptide in a cell, typically a LT-β-R- or NFκB1-encoding DNA is first introduced into a recombinant expression vector using standard molecular biology techniques, as described herein. A LT-β-R- or NFκB1-encoding DNA can be obtained, for example, by amplification using the polymerase chain reaction (PCR), using primers based on the LT-β-R or NFκB1 nucleotide sequence. Following isolation or amplification of LT-βR- or NFκB1-encoding DNA, the DNA fragment is introduced into an expression vector and transfected into target cells by standard methods, as described herein.

Other activating agents that can be used to stimulate the activity of a LT-β-R or NFκB1 polypeptide are chemical compounds that stimulate LT-β-R or NFκB1 activity in cells, such as compounds that directly stimulate LT-β-R or NFκB1 polypeptide and compounds that promote the interaction between LT-β-R or NFκB1 and target DNA or other polypeptides. Such compounds can be identified using screening assays that select for such compounds, as described above.

Nucleic acid molecules encoding LT-β-R and/or NFκB1 molecules may be introduced into the subject in a form suitable for expression of the encoded protein in the cells of the subject may also be used in the methods of the invention.

For example, a full length or partial cDNA sequence is cloned into a recombinant expression vector and the vector is transfected into a cell using standard molecular biology techniques. The cDNA can be obtained, for example, by amplification using the polymerase chain reaction (PCR) or by screening an appropriate cDNA library. The nucleotide sequences of the cDNA can be used for the design of PCR. primers that allow for amplification of a cDNA by standard PCR methods or for the design of a hybridization probe that can be used to screen a cDNA library using standard hybridization methods. Following isolation or amplification of the cDNA, the DNA fragment is introduced into a suitable expression vector.

In one embodiment of the invention, an activating agent is an agonistic binding molecule, i.e., a binding molecule that activates LT-β-R or NFκB1. For example, U.S. Pat. No. 6,312,691 and WO 96/22788, the contents of which are hereby incorporated in their entirety, describe methods and compositions for the treatment of cancer using LT-β-R agonistic binding molecules to trigger cancer cell death. For example, U.S. Pat. No. 6,312,691 describes LT-β-R agonists for use in the invention including membrane-bound LT-α/β complexes, soluble LT-α/β complexes and anti-LT-β-R binding molecules and methods for their preparation and purification.

In a one embodiment, the binding molecule is an antibody. Various forms of antibodies can be made using standard recombinant DNA techniques (Winter and Milstein, Nature, 349, pp. 293-99 (1991)).

In certain embodiments, the binding molecule may be a polyclonal antibody. For example, antibodies may be raised in mammals by multiple subcutaneous or intraperitoneal injections of the relevant antigen and an adjuvant. This immunization typically elicits an immune response that comprises production of antigen-reactive antibodies from activated splenocytes or lymphocytes. The resulting antibodies may be harvested from the serum of the animal to provide polyclonal preparations.

In another embodiment, the binding molecule is a monoclonal antibody. In certain embodiments, a monoclonal LT-β-R antibody of the invention may be selected from the group consisting of: BKA11, CDH10, BCG6, AGH1, BDA8, CBE11 and BHA10, each of which is described in WO 96/22788.

Anti-LT-β-R binding molecules monoclonal antibodies for use in the present invention may be produced in certain embodiments by a cell line selected from the group consisting of the cells lines in Table 1:

TABLE 1

| CELL LINE | mAb Name | Accession No. |
|---|---|---|
| a) AG.H1.5.1 | AGH1 | HB 11796 |
| b) BD.A8.AB9 | BDA8 | HB 11798 |
| c) BC.G6.AF5 | BCG6 | B 11794 |
| d) BH.A10 | BHA10 | B 11795 |
| e) BK.A11.AC10 | BKA11 | B 11799 |
| f) CB.E11.1 | CBE11 | B 11793 |
| g) CD.H10.1 | CDH10 | B 11797 |

The preparation of monoclonal antibodies is a well-known process (Kohler et al., Nature, 256:495 (1975)) in which the relatively short-lived, or mortal, lymphocytes from a mammal which has been injected with antigen are fused with an immortal tumor cell line (e.g. a myeloma cell line), thus, producing hybrid cells or “hybridomas” which are both immortal and capable of producing the genetically coded antibody of the B cell. The resulting hybrids are segregated into single genetic strains by selection, dilution, and regrowth with each individual strain comprising specific genes for the formation of a single antibody. They produce antibodies which are homogeneous against a desired antigen and, in reference to their pure genetic parentage, are termed “monoclonal.”

Hybridoma cells thus prepared are seeded and grown in a suitable culture medium that preferably contains one or more substances that inhibit the growth or survival of the unfused, parental myeloma cells. Those skilled in the art will appreciate that reagents, cell lines and media for the formation, selection and growth of hybridomas are commercially available from a number of sources and standardized protocols are well established. Generally, culture medium in which the hybridoma cells are growing is assayed for production of monoclonal antibodies against the desired antigen. Preferably, the binding specificity of the monoclonal antibodies produced by hybridoma cells is determined by immunoprecipitation or by an in vitro assay, such as a radioimmunoassay (RIA) or enzyme-linked immunoabsorbent assay (ELISA). After hybridoma cells are identified that produce antibodies of the desired specificity, affinity and/or activity, the clones may be subcloned by limiting dilution procedures and grown by standard methods (Goding, Monoclonal Antibodies: Principles and Practice, pp 59-103 (Academic Press, 1986)). It will further be appreciated that the monoclonal antibodies secreted by the subclones may be separated from culture medium, ascites fluid or serum by conventional purification procedures such as, for example, protein-A, hydroxylapatite chromatography, gel electrophoresis, dialysis or affinity chromatography.

In another embodiment, DNA encoding a desired monoclonal antibody may be readily isolated and sequenced using conventional procedures (e.g., by using oligonucleotide probes that are capable of binding specifically to genes encoding the heavy and light chains of murine antibodies). The isolated and subcloned hybridoma cells serve as a preferred source of such DNA. Once isolated, the DNA may be placed into expression vectors, which are then transfected into prokaryotic or eukaryotic host cells such as E. coli cells, simian COS cells, Chinese Hamster Ovary (CHO) cells or myeloma cells that do not otherwise produce immunoglobulins. More particularly, the isolated DNA (which may be modified as described herein) may be used to clone constant and variable region sequences for the manufacture antibodies as described in Newman et al., U.S. Pat. No. 5,658,570, filed Jan. 25, 1995, which is incorporated by reference herein. Essentially, this entails extraction of RNA from the selected cells, conversion to cDNA, and amplification by PCR using Ig specific primers. Suitable primers for this purpose are also described in U.S. Pat. No. 5,658,570. As will be discussed in more detail below, transformed cells expressing the desired antibody may be grown up in relatively large quantities to provide clinical and commercial supplies of the immunoglobulin.

Those skilled in the art will also appreciate that DNA encoding antibodies or antibody fragments may also be derived from antibody phage libraries, e.g., using pd phage or Fd phagemid technology. Exemplary methods are set forth, for example, in EP 368 684 B1; U.S. Pat. No. 5,969,108, Hoogenboom, H. R. and Chames. 2000. Immunol. Today 21:371; Nagy et al. 2002. Nat. Med. 8:801; Huie et al. 2001. Proc. Natl. Acad. Sci. USA 98:2682; Lui et al. 2002. J. Mol. Biol. 315:1063, each of which is incorporated herein by reference. Several publications (e.g., Marks et al. Bio/Technology 10:779-783 (1992)) have described the production of high affinity human antibodies by chain shuffling, as well as combinatorial infection and in vivo recombination as a strategy for constructing large phage libraries. In another embodiment, Ribosomal display can be used to replace bacteriophage as the display platform (see, e.g., Hanes et al. 2000. Nat. Biotechnol. 18:1287; Wilson et al. 2001. Proc. Natl. Acad. Sci. USA 98:3750; or Irving et al. 2001 J. Immunol. Methods 248:31. In yet another embodiment, cell surface libraries can be screened for antibodies (Boder et al. 2000. Proc. Natl. Acad. Sci. USA 97:10701; Daugherty et al. 2000 J. Immunol. Methods 243:211. Such procedures provide alternatives to traditional hybridoma techniques for the isolation and subsequent cloning of monoclonal antibodies.

Yet other embodiments of the present invention comprise the generation of human or substantially human antibodies in nonhuman animals, such as transgenic animals harboring one or more human immunoglobulin transgenes. Such animals may be used as a source for splenocytes for producing hybridomas, as is described in U.S. Pat. No. 5,569,825, WO00076310, WO00058499 and WO00037504 and incorporated by reference herein.

Yet another highly efficient means for generating recombinant antibodies is disclosed by Newman, Biotechnology, 10: 1455-1460 (1992). Specifically, this technique results in the generation of primatized antibodies that contain monkey variable domains and human constant sequences. This reference is incorporated by reference in its entirety herein. Moreover, this technique is also described in commonly assigned U.S. Pat. Nos. 5,658,570, 5,693,780 and 5,756,096 each of which is incorporated herein by reference.

In another embodiment, lymphocytes can be selected by micromanipulation and the variable genes isolated. For example, peripheral blood mononuclear cells can be isolated from an immunized mammal and cultured for about 7 days in vitro. The cultures can be screened for specific IgGs that meet the screening criteria. Cells from positive wells can be isolated. Individual Ig-producing B cells can be isolated by FACS or by identifying them in a complement-mediated hemolytic plaque assay. Ig-producing B cells can be micromanipulated into a tube and the Vh and VI genes can be amplified using, e.g., RT-PCR The VH and VL genes can be cloned into an antibody expression vector and transfected into cells (e.g., eukaryotic or prokaryotic cells) for expression.

Alternatively, antibody-producing cell lines may be selected and cultured using techniques well known to the skilled artisan. Such techniques are described in a variety of laboratory manuals and primary publications. In this respect, techniques suitable for use in the invention as described below are described in Current Protocols in Immunology, Coligan et al., Eds., Green Publishing Associates and Wiley-Interscience, John Wiley and Sons, New York (1991) which is herein incorporated by reference in its entirety, including supplements.

Variable and constant region domains can be obtained from any source, and be incorporated into a modified binding molecule of the invention. For example, to clone antibodies, mRNA can be isolated from hybridoma, spleen, or lymph cells, reverse transcribed into DNA, and antibody genes amplified by PCR. PCR may be initiated by consensus constant region primers or by more specific primers based on the published heavy and light chain DNA and amino acid sequences. As discussed above, PCR also may be used to isolate DNA clones encoding the antibody light and heavy chains. In this case the libraries may be screened by consensus primers or larger homologous probes, such as mouse constant region probes. Numerous primer sets suitable for amplification of antibody genes are known in the art (e.g., 5′ primers based on the N-terminal sequence of purified antibodies (Benhar and Pastan. 1994. Protein Engineering 7:1509); rapid amplification of cDNA ends (Ruberti, F. et al. 1994. J. Immunol. Methods 173:33); antibody leader sequences (Larrick et al. 1989 Biochem. Biophys. Res. Commun. 160:1250); or based on known variable region framework amino acid sequences from the Kabat (Kabat et al. 1991. Sequences of Proteins of Immunological Interest. Bethesda, Md. JS Dep. Health Hum. Serv. 5^{th} ed.) or the V-base databases (e.g., Orlandi et al. 1989. Proc. Natl. Acad. Sci. USA 86:3833; Sblattero et al. 1998. Immunotechnology 3:271; or Krebber et al. 1997. J. Immunol. Methods 201:35). Constant region domains can be selected having a particular effector function (or lacking a particular effector function) or with a particular modification to reduce immunogenicity. Variable and constant domains can be cloned, e.g., using the polymerase chain reaction and primers which are selected to amplify the domain of interest. PCR amplification methods are described in detail in U.S. Pat. Nos. 4,683,195; 4,683,202; 4,800,159; 4,965,188; and in, e.g., “PCR Protocols: A Guide to Methods and Applications” Innis et al. eds., Academic Press, San Diego, Calif. (1990); Ho et al. 1989. Gene 77:51; Horton et al. 1993. Methods Enzymol. 217:270).

Alternatively, V domains can be obtained from libraries of V gene sequences from an animal of choice. Libraries expressing random combinations of domains, e.g., VH and VL domains, can be screened with a desired antigen to identify elements which have desired binding characteristics. Methods of such screening are well known in the art. For example, antibody gene repertoires can be cloned into a λ bacteriophage expression vector (Huse, W D et al. 1989. Science 2476:1275). In addition, cells (Boder and Wittrup. 1997. Nat. Biotechnol. 15:553; Daugtherty, P. et al. 2000. J. Immunol. Methods. 243:211; Francisco et al. 1994. Proc. Natl. Acad. Sci. USA 90:10444; Georgiou et al. 1997. Nature Biotechnology 15:29) or viruses (e.g., Hoogenboom, H R. 1998 Immunotechnology 4:1 Winter et al. 1994. Annu. Rev. Immunol. 12:433; Griffiths, A D. 1998. Curr. Opin. Biotechnol. 9:102) expressing antibodies on their surface can be screened. Ribosomal display can also be used to screen antibody libraries (Hanes J., et al. 1998. Proc. Natl. Acad. Sci. USA 95:14130; Hanes, J. and Pluckthun. 1999. Curr. Top. Microbiol. Immunot 243:107; He, M. and Taussig. 1997. Nucleic Acids Research 25:5132).

Preferred libraries for screening are human V gene libraries. VL and VH domains from a non-human source may also be used. In one embodiment, such non-human V domains can be altered to reduce their immunogenicity using art recognized techniques.

Libraries can be naïve, from immunized subjects, or semi-synthetic (Hoogenboom, H. R. and Winter. 1992. J. Mol. Biol. 227:381; Griffiths, A D, et al. EMBO J. 13:3245; de Kruif, J. et al. 1995. J. Mol. Biol. 248:97; Barbas, C. F., et al. 1992. Proc. Natl. Acad. Sci. USA 89:4457).

In addition, the sequences of many antibody V and C domains are known and such domains can be synthesized using methods well known in the art. In one embodiment, mutations can be made to immunoglobulin domains to create a library of nucleic acid molecules having greater heterogeneity (Thompson, J., et al. 1996. J. Mol. Biol. 256:77; Lamminmaki, U. et al. 1999. J. Mol. Biol. 291:589; Caldwell, R. C. and Joyce G F. 1992. PCR Methods Appl. 2:28; Caldwell R C and Joyce G F. 1994. PCR Methods Appl. 3:S136. Standard screening procedures can be used to select high affinity variants. In another embodiment, changes to VH and VL sequences can be made to increase antibody avidity, e.g., using information obtained from crystal structures using techniques known in the art.

Antigen recognition sites or entire variable regions may be derived from one or more parental antibodies. The parental antibodies can include naturally occurring antibodies or antibody fragments, antibodies or antibody fragments adapted from naturally occurring antibodies, antibodies constructed de novo using sequences of antibodies or antibody fragments known to be specific for the LT-beta receptor or NFκB1. Sequences that may be derived from parental antibodies include heavy and/or light chain variable regions and/or CDRs, framework regions or other portions thereof.

In one embodiment, a binding molecule is a humanized antibody. To make humanized antibodies, animals are immunized with the desired antigen, the corresponding antibodies are isolated, and the portion of the variable region sequences responsible for specific antigen binding is removed. The animal-derived antigen binding regions are then cloned into the appropriate position of human antibody genes in which the antigen binding regions have been deleted. See, e.g. Jones, P. et al. (1986), Nature 321, 522-525 or Tempest et al. (1991) Biotechnology 9, 266-273. Also, transgenic mice, or other mammals, may be used to express humanized antibodies. Such humanization may be partial or complete. Humanized antibodies minimize the use of heterologous (inter-species) sequences in human antibodies, and are less likely to elicit immune responses in the treated subject. In one embodiment, humanized LT-β-R antibodies for use in the present invention may be produced in certain embodiments by a cell line selected from the group consisting of E46.4 (ATCC patent deposit designation PTA-3357) or cell line E77.4 (ATCC patent deposit designation 3765).

In one embodiments, the humanized LT-β-R antibody is humanized CBE11 (huCBE11) as described, including the nucleotide and amino acid sequence thereof, in PCT publication No WO 02/30986 and U.S. application Ser. No. 10/412,406. In another embodiment, the humanized LT-β-R antibody is humanized BHA10 (huBHA10), as described, including the nucleotide and amino acid sequence thereof, in PCT application no. PCT US03/20762 and U.S. Appin No. 11/021,819. Applicants' applications described above, the contents of which are hereby incorporated in their entirety, describe methods and compositions for the treatment of cancer using huCBE11 and huBHA10, to trigger cancer cell death.

In another embodiment, “chimeric” binding molecules can be constructed in which the antigen binding domain from an animal binding molecule is linked to a human constant domain (e.g. Cabilly et al., U.S. Pat. No. 4,816,567; Morrison et al., Proc. Natl. Acad. Sci. U.S.A., 81, pp. 6851-55 (1984)). Chimeric binding molecules reduce the observed immunogenic responses elicited by animal antibodies when used in human clinical treatments. Construction of different classes of recombinant binding molecules can also be accomplished by making chimeric or humanized binding molecules comprising the variable domains and human constant domains (CH1, CH2, CH3) isolated from different classes of immunoglobulins. For example, anti-LT-beta-R IgM binding molecules with increased antigen binding site valencies can be recombinantly produced by cloning the antigen binding site into vectors carrying the human .mu. chain constant regions (Arulanandam et al., J. Exp. Med., 177, pp. 1439-50 (1993); Lane et al., Eur. J. Immunol., 22, pp. 2573-78 (1993); Traunecker et al., Nature, 339, pp. 68-70 (1989)). In addition, standard recombinant DNA techniques can be used to alter the binding affinities of recombinant binding molecules with their antigens by altering amino acid residues in the vicinity of the antigen binding sites. See, e.g. (Queen et al., Proc. Natl. Acad. Sci. U.S.A., 86, pp. 10029-33 (1989); WO 94/04679).

Binding molecules of the invention may also be modified binding molecules. Exemplary modified binding molecules include, e.g., minibodies, diabodies, diabodies fused to CH3 molecules, tetravalent antibodies, intradiabodies (e.g., Jendreyko et al. 2003. Biol. Chem. 278:47813), bispecific antibodies, fusion proteins (e.g., antibody cytokine fusion proteins, proteins fused to at least a portion of an Fc receptor), bispecific antibodies. Other immunoglobulins (Ig) and certain variants thereof are described, for example in U.S. Pat. No. 4,745,055; EP 256,654; Faulkner et al., Nature 298:286 (1982); EP 120,694; EP 125,023; Morrison, J. Immun. 123:793 (1979); Kohler et al., Proc. Natl. Acad. Sci. USA 77:2197 (1980); Raso et al., Cancer Res. 41:2073 (1981); Morrison et al., Ann. Rev. Immunuol. 2:239 (1984); Morrison, Science 229:1202 (1985); Morrison et al., Proc. Natl. Acad. Sci. USA 81:6851 (1984); EP 255,694; EP 266,663; and WO 88/03559. Reassorted immunoglobulin chains also are known. See, for example, U.S. Pat. No. 4,444,878; WO 88/03565; and EP 68,763 and references cited therein.

In one embodiment, a binding molecule of the invention comprises an immunoglobulin heavy chain having deletion or substitution of at least one amino acid compared to wild type. For example, the mutation of one or more single amino acid in selected areas of the CH2 domain may be enough to substantially reduce Fc binding and thereby increase tumor localization. Similarly, it may be desirable to simply delete that part of one or more constant region domains that control the effector function (e.g. complement binding) to be modulated. Such partial deletions of the constant regions may improve selected characteristics of the antibody (serum half-life) while leaving other desirable functions associated with the subject constant region domain intact. Accordingly, in one embodiment, a binding molecule of the invention lacks all or part of a CH2 domain. Moreover, the constant regions of the binding molecules of the invention may be modified through the mutation or substitution of one or more amino acids that enhances the profile of the resulting construct. In this respect it may be possible to disrupt the activity provided by a conserved binding site (e.g. Fc binding) while substantially maintaining the configuration and immunogenic profile of the modified binding molecule. Yet other preferred embodiments may comprise the addition of one or more amino acids to the constant region to enhance desirable characteristics such as effector function or provide for more cytotoxin or carbohydrate attachment. In such embodiments it may be desirable to insert or replicate specific sequences derived from selected constant region domains.

In another embodiment, mutations to naturally occurring hinge regions can be made. Such modifications to the constant region in accordance with the instant invention may easily be made using well known biochemical or molecular engineering techniques well within the skill of the art.

In one embodiment, a binding molecule of the invention comprises modified constant regions wherein one or more domains are partially or entirely deleted (“domain deleted antibodies”). In especially preferred embodiments compatible modified binding molecules will comprise domain deleted constructs or variants wherein the entire CH2 domain has been removed.

In one embodiment, the modified binding molecules of the invention are minibodies. Minibodies are dimeric molecules made up of two polypeptide chains each comprising an ScFv molecule (a single polypeptide comprising one or more antigen binding sites, e.g., a VL domain linked by a flexible linker to a VH domain fused to a CH3 domain via a connecting peptide.

ScFv molecules can be constructed in a VH-linker-VL orientation or VL-linker-VH orientation.

The flexible hinge that links the VL and VH domains that make up the antigen binding site preferably comprises from about 10 to about 50 amino acid residues, see, e.g., Huston et al. 1988. Proc. Natl. Acad. Sci. USA 85:5879.

Methods of making single chain antibodies are well known in the art, e.g., Ho et al. 1989. Gene 77:51; Bird et al. 1988 Science 242:423; Pantoliano et al. 1991. Biochemistry 30:10117; Milenic et al. 1991. Cancer Research 51:6363; Takkinen et al. 1991. Protein Engineering 4:837.

Minibodies can be made by constructing an ScFv component and connecting peptide-CH3 component using methods described in the art (see, e.g., U.S. Pat. No. 5,837,821 or WO 94/09817A1). These components can be isolated from separate plasmids as restriction fragments and then ligated and recloned into an appropriate vector. Appropriate assembly can be verified by restriction digestion and DNA sequence analysis.

In another embodiment, a tetravalent minibody can be constructed. Tetravalent minibodies can be constructed in the same manner as minibodies, except that two ScFv molecules are linked using a flexible linker.

In another embodiment, the modified antibodies of the invention are CH2 domain deleted antibodies. Domain deleted constructs can be derived from a vector (e.g., from IDEC Pharmaceuticals, San Diego) encoding an IgG₁ human constant domain (see, e.g., WO 02/060955A2 and WO02/096948A2).

Besides the deletion of whole constant region domains, it will be appreciated that the antibodies of the present invention can be engineered to partially delete or substitute of a few amino acids or even a single amino acid. For example, the mutation of a single amino acid in selected areas of the C_{H}2 domain may be enough to substantially reduce Fc binding and thereby increase tumor localization. Similarly, it may be desirable to simply delete that part of one or more constant region domains that control the effector function (e.g. complement C1Q binding). Such partial deletions of the constant regions may improve selected characteristics of the antibody (serum half-life) while leaving other desirable functions associated with the subject constant region domain intact.

Creation of a C_{H}2 domain deleted version can be accomplished by way of overlapping PCR mutagenesis. The gamma 1 constant domain begins with a plasmid encoded Nhe I site with is in translational reading frame with the immunoglobulin sequence. A 5′ PCR primer was constructed encoding the Nhe I site as well as sequence immediately downstream. A 3′ PCR primer mate was constructed such that it anneals with the 3′ end to the immunoglobulin hinge region and encodes in frame the first several amino acids of the gamma 1 CH3 domain. A second PCR primer pair consisted of the reverse complement of the 3′ PCR primer from the first pair (above) as the 5′ primer and a 3′ primer that anneals at a locus spanning the BsrG I restriction site within the C_{H}3 domain. Following each PCR amplification, the resultant products were utilized as template with the Nhe I and BsrG I 5′ and 3′, respectively primers. The amplified product was then cloned back into N5KG1 to create the plasmid N5KG1ΔC_{H}2. This construction places the intact CH3 domain immediately downstream and in frame with the intact hinge region. A similar procedure can be used to create a domain deleted construct in which the CH3 domain is immediately downstream of a connecting peptide. For example, a domain deleted version of the C2B8 antibody was created in this manner as described in U.S. Pat. Nos. 5,648,267 and 5,736,137 each of which is incorporated herein by reference.

In one embodiment, tetravalent domain-deleted antibodies can be produced by combining a DNA sequence encoding a domain deleted antibody with a ScFv molecule. For example, in one embodiment, these sequences are combined such that the ScFv molecule is linked at its N-terminus to the CH3 domain of the domain deleted antibody via a flexible linker.

In another embodiment a tetravalent antibody can be made by fusing an ScFv molecule to a connecting peptide, which is fused to a CH1 domain to construct an ScFv-Fab tetravalent molecule. (Coloma and Morrison. 1997. Nature Biotechnology. 15:159; WO 95/09917).

In another embodiment, the modified antibodies of the invention are diabodies. Diabodies are similar to scFv molecules, but usually have a short (less than 10 and preferably 1-5) amino acid residue linker connecting both V-domains, such that the VL and VH domains on the same polypeptide chain cannot interact. Instead, the VL and VH domain of one polypeptide chain interact with the VH and VL domain (respectively) on a second polypeptide chain (WO 02/02781). In one embodiment, a binding molecule of the invention is a diabody fused to at least one heavy chain portion. In a preferred embodiment, a binding molecule of the invention is a diabody fused to a CH3 domain.

In one embodiment a modified antibody of the invention comprises a tetravalent or bispecific tetravalent CH2 domain-deleted antibody with a scFv appended to the N-terminus of the light chain. In another embodiment of the invention, a binding molecule comprises a a tetravalent or bispecific tetravalent CH2 domain-deleted antibody with a scFv appended to the N-terminus of the heavy chain. In one embodiment, the attachment of the scFv to the N-terminus results in reduced aggregation of the molecules as compared to molecules in which the scFv is attached at the carboxy-terminus. Other forms of modified binding molecules are also within the scope of the instant invention (e.g., WO 02/02781 A1; U.S. Pat. Nos. 5,959,083; 6,476,198 B1; US 2002/0103345 A1; WO 00/06605; Byrn et al. 1990. Nature. 344:667-70; Chamow and Ashkenazi. 1996. Trends Biotechnol. 14:52).

In still other embodiments, the binding molecule is a multivalent antibody. In one embodiment, a multivalent antibody comprises at least one antigen recognition site specific for, e.g., a LT-β-R or NFκB1 epitope. In certain embodiments, at least one of the antigen recognition sites is located within a scFv domain, while in other embodiments all antigen recognition sites are located within scFv domains

Binding molecules may be bivalent, trivalent, tetravalent or pentavalent. In certain embodiments, the binding molecule is monospecific. In one embodiment, a LT-β-R binding molecule is specific for the epitope to which CBE11 binds. In other embodiments, the LT-β-R binding molecule is a monospecific tetravalent LT-β-R agonist antibody comprising four CBE11-antigen recognition sites. In another embodiment, the LT-β-R binding molecule is specific for the BHA10 epitope, and, in some embodiments, is tetravalent. In any of these embodiments, at least one antigen recognition site may be located on a scFv domain, and in certain of these embodiments, all antigen recognition sites may be located on scFv domains. Binding molecules may be multispecific.

In certain embodiments, a multivalent binding molecule may be multispecific, i.e., has at least one binding site that binds to, e.g., LT-β-R, NFκB1, or an epitope of LT-βR or NFκB1 and at least one second binding site that binds to a second, different molecule or to a second, different epitope of e.g., LT-β-R or NFκB1.

Multivalent, multispecific binding molecules may contain a heavy chain comprising two or more variable regions and/or a light chain comprising one or more variable regions wherein at least two of the variable regions recognize different epitopes on the, e.g., LT-beta receptor.

In one embodiment, the multivalent binding molecule is an agonist of the lymphotoxin-beta receptor and comprises at least two domains that are capable of binding to the receptor and inducing LT-β-R and/or NFκB1 signaling. These constructs can include a heavy chain containing two or more variable regions comprising antigen recognitions sites specific for binding the LT-beta receptor and a light chain containing one or more variable regions or can be constructed to comprise only heavy chains or light chains containing two or more variable regions comprising CDRs specific for binding the, e.g., LT-beta receptor. Examples of multivalent molecules that may be used in the methods and compositions of the invention are described in WO 200405819, incorporated by reference herein.

In one embodiments of the invention, the binding molecule is specific for at least two members of the group of lymphotoxin-beta receptor (LT-β-R) epitopes consisting of the epitopes to which one of following antibodies bind: BKA11, CDH10, BCG6, AGH1, BDA8, CBE11 and BHA10. In one embodiment, a LT-β-R binding molecule is specific for the epitope to which the CBE11 and BHA10 antibodies bind, and in certain embodiments, is tetravalent. In one embodiment, the LT-β-R binding molecule has two CBE11-specific antigen recognition sites and two BHA10-specific recognition sites, wherein the binding molecule is a bispecific tetravalent LT-β-R agonist binding molecule. In any of the multispecific binding molecules, at least one antigen recognition site may be located on a scFv domain, and in certain embodiments, all antigen recognition sites are located on scFv domains.

In certain embodiments, the binding molecule is bispecific. Bispecific molecules can bind to two different target sites, e.g., on the same target molecule or on different target molecules. For example, in the case of antibodies, bispecific molecules can bind to two different epitopes, e.g., on the same antigen or on two different antigens. Bispecific molecules can also be used for human therapy, e.g., by directing cytotoxicity to a specific target (for example by binding to a pathogen or tumor cell and to a cytotoxic trigger molecule, such as the T cell receptor or the Fcγ receptor. Bispecific antibodies can also be used, e.g., as fibrinolytic agents or vaccine adjuvants.

In one embodiment, the bispecific binding molecules of the invention include those with at least one arm (ie. binding site) directed against LT-β-R and at least one arm directed against a cell-surface molecule or a soluble molecule. Exemplary cell-surface molecules include receptors or tumor cell antigens that are overexpressed on the surface of a tumor or neoplastic cell. Exemplary soluble molecules include anti-tumor agents (e.g., toxins, chemotherapeutics, and prodrugs thereof) and soluble enzymes (e.g. prodrug converting enzymes).

In one embodiment, the soluble molecule to which a LT-β-R bispecific binding molecule binds is a soluble ligand of the TNF family, in addition to LT-β-R. Examples of TNF family ligands include, but are not limited to, LTA (which binds TNFR1/TNFRSF1A), TNF (which binds CD120b/INFRSF1B), LTB (which binds LTBR/TNFRSF3), OX40L (which binds OX40/TNFRSF4), CD40L (which binds CD40/TNFRSF5), (which binds Fas/TNFRSF6 and DcR3/TNFRSF6B), CD27L (which binds CD27/TNFRSF7), CD30L (which binds CD30/TNFRSF8), 4-1-BB-L (which binds 4-1-BB/TNFRSF9), TRAIL (which binds TRAIL-R1/TNFRSF10A, TRAIL-R2/TNFRSF10B, TRAIL-R3/TNFRSF10C, and TRAIL-R4/TNFRSF10D), RANKL (which binds RANK/TNFRSF11A and Osteoprotegrin/TNFRSF11B), APO-3L (which binds APO-3/TNFRSF12 and DR3L/TNFRSF12L), APRIL (which binds TACl/INFRSF13B), BAFF (which binds BAFFRJTNFRSF13A), LIGHT (which binds HVEM/TNFRSF14), NGF ligands (which bind LNGFR, e.g. NGF-β, NGF-2/NTF3, NTFS, BDNF, IFRD1), GITRL (which binds GITRJTNFRSF18), EDAR1 & XEDAR ligand, Fn14 ligand, and Troy/Trade ligand.

In another embodiment, the soluble molecule to which a LT-β-R bispecific binding molecule of the invention binds is another receptor of the TNF family, i.e., a TNF receptor other than LT-β-R. The limiting factor in the treatment of tumors with monospecific TNFR binding molecules is that often only a subset of tumors appears to be sensitive to such therapies. Bispecific TNFR binding molecules can specifically activate TNFRs, and enhance receptor signaling by, for example, bringing the TNFRs into close proximity which can thus target more than one TNFR or TNFR type and enhance signaling, thus providing an improved method of treating cancer. In one embodiment, the bispecific TNFR binding molecule increases the signal strength by binding to two or more TNFRs of the same type increasing the number of TNFRs being brought together. In another more preferred embodiment, the bispecific TNFR binding molecule is capable of binding to two different receptors of the TNF family.

In one embodiment, the LT-β-R bispecific binding molecule binds LT-β-R and a TNFR that contains a death domain. The term “death domain” refers to a cytoplasmic region of a TNF family receptor which is involved TNF-mediated cell death or apoptotic signaling and cell-cytotoxicity induction mediated by these receptors. This region couples the receptor to caspase activation via adaptor proteins resulting in activation of the extrinsic death pathway. Examples of TNF receptors which contain death domains include, but are not limited to, TNFR1 (TNFRSFIA), Fas (TNFRSF6), DR-3 (TNFRSF6B), LNGFR (TNFRSF16) TRAIL-R1 (TNFRSF10A), TRAIL-R2 (TNFRSF10B) and DR6 (TNFRSF21). The apoptotic signaling of these receptors is modulated upon binding of a cognate ligand and formation of any of the following receptor-ligand pairs: TNFR1/TNFα, Fas/FasL, DR-3/DR-3LG, TRAIL-R1/TRAIL, or TRAIL-R2/TRAIL.

Bispecific binding molecules that target TNF family receptors containing death domains are useful for the treatment of cancer since the TNFRs of this type are often overexpressed on tumor cells and stimulating of the receptor can activate tumor cell apoptosis. In preferred embodiments, the death-domain containing TNFR to which the bispecific binding molecule of the invention binds is TRAIL-R2. TRAIL-R2 is preferred for human tumor therapy since its activation does not trigger hepatocyte apoptosis and hence should have reduced toxicity.

While the activation of some of death domain containing receptors, e.g. TNFR1 or Fas, has been toxic in in vivo applications, it is likely that tethering these receptors to other TNF receptors may diminish toxicity and thus render a toxic antibody less toxic.

A number of antibodies have been generated to death domain containing TNF receptors and are well known in the art. Such antibodies include anti-TNF-R1 monoclonal antibodies (R&D systems anti-TNF-R1; Tularik mAb #985, U.S. Pat. Nos. 6,110,690; 6,437,113), anti-Fas receptor mAb CH-11 (U.S. Pat. No. 6,312,691; WO 95/10540), anti-DR3 antibodies (U.S. Pat. No. 5,985,547; Johnson, et al. (1984) ImmunoBiology of HLA, ed. Dupont, B. O., Springer, New York; U.S. Pat. Nos. 6,462,176; 6,469,166), and anti-TRAIL-R antibodies (U.S. Pat. Nos. 5,763,223; 6,072,047; 6,284,236; 6,521,228; 6,569,642; 6,642,358; and U.S. Pat. No. 6,417,328).

Other target TNF family receptors with a role in tumor formation can be identified using existing RNA databases of receptor expression in various cell types which allow one to define TNF family receptors that are present or ideally overexpressed on various tumors. Moreover, existing RNA databases provide an additional advantage in that the pair of TNF family receptors to which a bispecific TNFR binding molecule of the invention binds could be optimized by identifying those receptor pairs that are more uniquely expressed on a tumor type or subset of tumors but are not abundant on normal tissues, especially liver and vasculature. In such a manner receptor pairs (or more) are identified that could deliver a potent signal to the tumor and spare normal tissues.

The multispecific binding molecules of the invention may be monovalent for each specificity or multivalent for each specificity. In one embodiment, a bispecific binding molecule of the invention may comprise one binding site that reacts with a first target molecule, e.g., LT-β-R, and one binding site that reacts with a second target molecule (e.g. a bispecific antibody molecule, fusion protein, or minibody). In another embodiment, a bispecific binding molecule of the invention may comprise two binding sites that react with a first target molecule, e.g., LT-β-R, and two binding sites that react with a second target molecule (e.g. a bispecific scFv2 tetravalent antibody, tetravalent minibody, or diabody).

In one embodiment, at least one binding site of a multispecific binding molecule of the invention is an antigen binding region of an anti-LT-β-R antibody, or an antigen binding fragment thereof.

In another embodiment, at least one binding site of multispecific binding molecule is a single chain Fv fragment. In one embodiment, the multispecific binding molecules of the invention are bivalent minibodies with one arm containing a scFv fragment directed to a first target molecule, e.g., LT-β-R, and a second arm containing a scFv directed to a second target molecule.

In another embodiment, the multispecific binding molecules of the invention are scFv tetravalent minibodies, with each heavy chain portion of the scFv tetravalent minibody containing first and second scFv fragments. Said second scFv fragment may be linked to the N-terminus of the first scFv fragment (e.g. bispecific N_{H} scFv tetravalent minibodies or bispecific N_{L} scFv tetravalent minibodies). Alternatively, the second scFv fragment may be linked to the C-terminus of said heavy chain portion containing said first scFv fragment (e.g. bispecific C-scFv tetravalent minibodies). In one embodiment, the first and second scFv fragments of may bind the same or different target molecule. Where the first and second scFv fragments of a first heavy chain portion of a bispecific tetravalent minibody bind the same target molecule, at least one of the first and second scFv fragments of the second heavy chain portion of the bispecific tetravalent minibody binds a different target molecule.

In another embodiment, the multispecific binding molecules of the invention are bispecific diabodies, with each arm of the diabody comprising tandem scFv fragments. In one embodiment, a bispecific diabody may comprise a first arm with a first binding specificity and a second arm with a second binding specificity. In another embodiment, each arm of the diabody may comprise a first scFv fragment with a first binding specificity and a second scFv fragment with a second binding specificity.

In another embodiment, the multispecific binding molecules of the invention are scFv2 tetravalent antibodies with each heavy chain portion of the scFv2 tetravalent antibody containing a scFv fragment. The scFv fragments may be linked to the N-termini of a variable region of the heavy chain portions (e.g. bispecific Nₐ scFv2 tetravalent antibodies or bispecific N_{L} scFv2 tetravalent antibodies). Alternatively, the scFv fragments may be linked to the C-termini of the heavy chain portions of the scFv2 tetravalent antibody (e.g. bispecific C-scFv2 tetravalent antibodies. Each heavy chain portion of the scFv2 tetravalent antibody may have variable regions and scFv fragments that bind the same or different target molecules. Where the scFv fragment and variable region of a first heavy chain portion of a bispecific scFc2 tetravalent antibody bind the same target molecule, at least one of the fust and second scFv fragments of the second heavy chain portion of the bispecific tetravalent minibody binds a different target molecule.

In another embodiment, the multispecific binding molecules of the invention are scFv2 tetravalent domain-deleted antibodies with each heavy chain portion of the scFv2 tetravalent antibody containing a scFv fragment. The scFv fragments may be linked to the N-termini of a variable region of the heavy chain portions (e.g. bispecific N_{H} scFv2 tetravalent domain-deleted antibodies or bispecific N_{L} scFv2 tetravalent antibodies. Alternatively, the scFv fragments may be linked to the C-termini of the heavy chain portions of the scFv2 tetravalent antibody (e.g. bispecific C-scFv2 tetravalent antibodies).

Methods for making multivalent multispecific antibodies are known in the art. Traditional production of full length bispecific antibodies is based on the coexpression of two immunoglobulin heavy chain-light chain pairs, where the two chains have different specificities (Milstein et al., Nature, 305:537-539 (1983)). Because of the random assortment of immunoglobulin heavy and light chains, these hybridomas (quadromas) produce a potential mixture of 10 different antibody molecules, of which only one has the correct bispecific structure. Purification of the correct molecule, which is usually done by affinity chromatography steps, is rather cumbersome, and the product yields are low. Similar procedures are disclosed in WO 93/08829, and in Traunecker et al., EMBO J., 10:3655-3659 (1991).

Multivalent antibodies may be constructed in a variety different ways using a variety of different sequences derived from parental antibodies. In one embodiment, a parental antibody is a LT-β-R antibody and includes murine or humanized BHA10 (Browning et al., J. Immunol. 154: 33 (1995); Browning et al. J. Exp. Med. 183:867 (1996)) and/or murine or humanized CBE11 (U.S. Pat. No. 6,312,691).

Methods of producing bispecific molecules are well known in the art. For example, recombinant technology can be used to produce bispecific molecules, e.g., diabodies, single-chain diabodies, tandem scFvs, etc. Exemplary techniques for producing bispecific molecules are known in the art (e.g., Kontermann et al. Methods in Molecular Biology Vol. 248: Antibody Engineering: Methods and Protocols. Pp 227-242 US 2003/0207346 A1 and the references cited therein). In one embodiment, a multimeric bispecific molecules are prepared using methods such as those described e.g., in US 2003/0207346 A1 or U.S. Pat. No. 5,821,333, or US2004/0058400.

In another embodiment, a multispecific binding molecule of the invention is a multispecific fusion protein. As used herein the phrase “multispecific fusion protein” designates fusion proteins having at least two binding specificities (i.e. combining two or more binding domains. Multispecific fusion proteins can be assembled as heterodimers, heterotrimers or heterotetramers, essentially as disclosed in WO 89/02922 (published Apr. 6, 1989), in EP 314, 317 (published May 3, 1989), and in U.S. Pat. No. 5,116,964 issued May 2, 1992. Preferred multispecific fusion proteins are bispecific.

In one embodiment, the subject bispecific molecule is expressed in an expression system used to express antibody molecules, for example mammalian cells, yeast such as Picchia, E. coli, Bacculovirus, etc. In one embodiment, the subject bispecific molecule is expressed in the NEOSPLA vector system (see, e.g., U.S. Pat. No. 6,159,730). This vector contains the cytomegalovirus promoter/enhancer, the mouse beta globin major promoter, the SV40 origin of replication, the bovine growth hormone polyadenylation sequence, neomycin phosphotransferase exon 1 and exon 2, the dihydrofolate reductase gene and leader sequence.

A variety of other multivalent antibody constructs may be developed by one of skill in the art using routine recombinant DNA techniques; for example as described in PCT International Application No. PCT/US86/02269; European Patent Application No. 184,187; European Patent Application No. 171,496; European Patent Application No. 173,494; PCT International Publication No. WO 86/01533; U.S. Pat. No. 4,816,567; European Patent Application No. 125,023; Better et al. (1988) Science 240:1041-1043; Liu et al. (1987) Proc. Natl Acad. Sci. USA 84:3439-3443; Liu et al. (1987) J. Immunol. 139:3521-3526; Sun et al. (1987) Proc. Natl. Acad. Sci. USA 84:214-218; Nishimura et al. (1987) Cancer Res. 47:999-1005; Wood et al. (1985) Nature 314:446-449; Shaw et al. (1988) J. Natl. Cancer Inst. 80:1553-1559); Morrison (1985) Science 229:1202-1207; Oi et al. (1986) Bio Techniques 4:214; U.S. Pat. No. 5,225,539; Jones et al. (1986) Nature 321:552-525; Verhoeyan et al. (1988) Science 239:1534; Beidler et al. (1988) J. Immunol. 141:4053-4060; and Winter and Milstein, Nature, 349, pp. 293-99 (1991)). Preferably non-human antibodies are “humanized” by linking the non-human antigen binding domain with a human constant domain (e.g. Cabilly et al, U.S. Pat. No. 4,816,567; Morrison et al, Proc. Nail Acad. Sci. U.S.A., 81, pp. 6851-55 (1984)).

Other methods which may be used to prepare multivalent antibody constructs are descn'bed in the following publications: Ghetie, Maria-Ana et al. (2001) Blood 97:1392-1398; Wolff, Edith A. et al. (1993) Cancer Research 53:2560-2565; Ghetie, Maria-Ana et al. (1997) Proc. Natl. Acad. Sci. 94:7509-7514; Kim, J. C. et al. (2002) Int. J. Cancer 97(4):542-547; Todorovska, Aneta et al. (2001) Journal of Immunological Methods 248:47-66; Coloma M. J. et al. (1997) Nature Biotechnology 15:159-163; Zuo, Zhuang et al. (2000) Protein Engineering (Suppl.) 13(5):361-367; Santos A. D., et al. (1999) Clinical Cancer Research 5:3118s-3123s; Presta, Leonard G. (2002) Current Pharmaceutical Biotechnology 3:237-256; van Spriel, Annemiek et al., (2000) Review Immunology Today 21(8) 391-397.

In some embodiments, the binding molecules and binding molecule fragments of the invention may be chemically modified to provide a desired effect. For example, pegylation of antibodies and antibody fragments of the invention may be carried out by any of the pegylation reactions known in the art, as described, for example, in the following references: Focus on Growth Factors 3:4-10 (1992); EP 0 154 316; and EP 0 401 384 (each of which is incorporated by reference herein in its entirety). Preferably, the pegylation is carried out via an acylation reaction or an alkylation reaction with a reactive polyethylene glycol molecule (or an analogous reactive water-soluble polymer). A preferred water-soluble polymer for pegylation of the binding molecules and binding molecule fragments of the invention is polyethylene glycol (PEG). As used herein, “polyethylene glycol” is meant to encompass any of the forms of PEG that have been used to derivative other proteins, such as mono (Cl-ClO) alkoxy- or aryloxy-polyethylene glycol.

Methods for preparing pegylated binding molecules and binding molecule fragments of the invention will generally comprise the steps of (a) reacting the binding molecule or binding molecule fragment with polyethylene glycol, such as a reactive ester or aldehyde derivative of PEG, under conditions whereby the binding molecule or binding molecule fragment becomes attached to one or more PEG groups, and (b) obtaining the reaction products. It will be apparent to one of ordinary skill in the art to select the optimal reaction conditions or the acylation reactions based on known parameters and the desired result.

Pegylated binding molecules and binding molecule fragments may generally be used to treat conditions that may be alleviated or modulated by administration of the binding molecules and binding molecule fragments described herein. Generally the pegylated binding molecules and binding molecule fragments have increased half-life, as compared to the nonpegylated binding molecules and binding molecule fragments. The pegylated binding molecules and binding molecule fragments may be employed alone, together, or in combination with other pharmaceutical compositions.

In other embodiments of the invention the binding molecules or antigen-binding fragments thereof are conjugated to albumen using an recognized techniques.

In another embodiment of the invention, binding molecules, or fragments thereof, are modified to reduce or eliminate potential glycosylation sites. Such modified antibodies are often referred to as “aglycosylated” binding molecules. In order to improve the binding affinity of a binding molecule or antigen-binding fragment thereof, glycosylation sites of the binding molecule can be altered, for example, by mutagenesis (e.g., site-directed mutagenesis). “Glycosylation sites” refer to amino acid residues which are recognized by a eukaryotic cell as locations for the attachment of sugar residues. The amino acids where carbohydrate, such as oligosaccharide, is attached are typically asparagine (N-linkage), serine (O-linkage), and threonine (O-linkage) residues. In order to identify potential glycosylation sites within an binding molecule or antigen-binding fragment, the sequence of the binding molecule is examined, for example, by using publicly available databases such as the website provided by the Center for Biological Sequence Analysis (see http://www.cbs.dtu.dk/services/NetNGlyc/ for predicting N-linked glycoslyation sites) and http://www.cbs.dtu.dk/services/NetOGlyc/ for predicting O-linked glycoslyation sites). Additional methods for altering glycosylation sites of binding molecules are described in U.S. Pat. Nos. 6,350,861 and 5,714,350.

In yet another embodiment of the invention, binding molecules or antigen binding fragments thereof can be altered wherein the constant region of the binding molecule is modified to reduce at least one constant region-mediated biological effector function relative to an unmodified binding molecule. To modify a binding molecule of the invention such that it exhibits reduced binding to the Fc receptor (FcR), the immunoglobulin constant region segment of the binding molecule can be mutated at particular regions necessary for FcR interactions (see e.g., Canfield et al (1991) J. Exp. Med 173:1483; and Lund, J. et al. (1991) J. of Immunol. 147:2657). Reduction in FcR binding ability of the binding molecule may also reduce other effector functions which rely on FcR interactions, such as opsonization and phagocytosis and antigen-dependent cellular cytotoxicity.

In a particular embodiment the invention further features binding molecules having altered effector function, such as the ability to bind effector molecules, for example, complement or a receptor on an effector cell. In particular, the humanized binding molecules of the invention have an altered constant region, e.g., Fc region, wherein at least one amino acid residue in the Fc region has been replaced with a different residue or side chain thereby reducing the ability of the binding molecule to bind the FcR. Reduction in FcR binding ability of the binding molecule may also reduce other effector functions which rely on FcR interactions, such as opsonization and phagocytosis and antigen-dependent cellular cytotoxicity. In one embodiment, the modified humanized binding molecule is of the IgG class, comprises at least one amino acid residue replacement in the Fc region such that the humanized binding molecule has an altered effector function, e.g., as compared with an unmodified humanized binding molecule. In particular embodiments, the humanized binding molecule of the invention has an altered effector function such that it is less immunogenic (e.g., does not provoke undesired effector cell activity, lysis, or complement binding), and/or has a more desirable half-life while retaining specificity for LTβR or a ligand thereof.

Alternatively, the invention features humanized binding molecules having altered constant regions to enhance FcR binding, e.g., FcγR3 binding. Such binding molecules are useful for modulating effector cell function, e.g., for increasing ADCC activity, e.g., particularly for use in oncology applications of the invention.

As used herein, “antibody-dependent cell-mediated cytotoxicity” and “ADCC” refer to a cell-mediated reaction in which nonspecific cytotoxic cells that express FcRs (e.g. Natural Killer (NK) cells, neutrophils, and macrophages) recognize bound binding molecule on a target cell and subsequently cause lysis of the target cell. The primary cells for mediating ADCC, NK cells, express FcγRIII only, whereas monocytes express FcγRI, FcγRII and FcγRIII of the antibody, e.g., a conjugate of the binding molecule and another agent or binding molecule.

In one embodiment, binding molecules of the invention can be conjugated to a chemotherapeutic agent or a toxin for use in the methods of the invention. Exemplary chemotherapeutics that can be conjugated to the antibodies of the present invention include, but are not limited to radioconjugates (90Y, 131I, 99mTc, 111In, 186Rh, et al.).

The cytotoxic effects of binding molecules on a tumor may be enhanced by the presence of an activating agent, particularly IFN-gamma. For example, clinical experiments have demonstrated interferon induction by double stranded RNA (dsRNA) treatment. Accordingly, polyriboguanylic/polyribocytidylic acid (poly-rG/rC) and other forms of dsRNA are effective as interferon inducers (Juraskova et al., Eur. J. Pharmacol., 221, pp. 107-11 (1992)).

The binding molecules produced as described above may be purified to a suitable purity for use as a pharmaceutical composition. Generally, a purified composition will have one species that comprises more than about 85 percent of all species present in the composition, more than about 85%, 90%, 95%, 99% or more of all species present. The object species may be purified to essential homogeneity (contaminant species cannot be detected in the composition by conventional detection methods) wherein the composition consists essentially of a single species. A skilled artisan may purify a polypeptide of the invention using standard techniques for protein purification in light of the teachings herein. Purity of a polypeptide may be determined by a number of methods known to those of skill in the art, including for example, amino-terminal amino acid sequence analysis, gel electrophoresis and mass-spectrometry analysis.

### B. Inhibitory Agents

In one embodiment, an inhibitory agent of the invention is an antisense nucleic acid molecule that is complementary to a gene encoding TRAF3, or to a portion of said gene, or a recombinant expression vector encoding said antisense nucleic acid molecule. As used herein, an “antisense” nucleic acid comprises a nucleotide sequence which is complementary to a “sense” nucleic acid encoding a protein, e.g., complementary to the coding strand of a double-stranded cDNA molecule, complementary to an mRNA sequence or complementary to the coding strand of a gene. Accordingly, an antisense nucleic acid molecule can hydrogen bond to a sense nucleic acid.

In one embodiment, an inhibitory agent is an siRNA molecule, e.g., of a TRAF3 molecule. In one embodiment, a biologic agent that inhibits angiogenesis mediates RNAi. RNA interference (RNAi) is a post-transcriptional, targeted gene-silencing technique that uses double-stranded RNA (dsRNA) to degrade messenger RNA (mRNA) containing the same sequence as the dsRNA (Sharp, P. A. and Zamore, P. D. 287, 2431-2432 (2000); Zamore, P. D., et al. Cell 101, 25-33 (2000). Tuschl, T. et al. Genes Dev. 13, 3191-3197 (1999); Cottrell T R, and Doering T L. 2003. Trends Microbiol. 11:37-43; Bushman F. 2003. Mol. Therapy. 7:9-10; McManus M T and Sharp P A. 2002. Nat Rev Genet. 3:737-47). The process occurs when an endogenous ribonuclease cleaves the longer dsRNA into shorter, e.g., 21- or 22-nucleotide-long RNAs, termed small interfering RNAs or siRNAs. The smaller RNA segments then mediate the degradation of the target mRNA. Kits for synthesis of RNAi are commercially available from, e.g. New England Biolabs or Ambion. In one embodiment one or more of the chemistries described herein for use in antisense RNA can be employed in molecules that mediate RNAi.

The use of antisense nucleic acids to downregulate the expression of a particular protein in a cell is well known in the art (see e.g., Weintraub, H. et al., Antisense RNA as a molecular tool for genetic analysis, Reviews—Trends in Genetics, Vol. 1(1) 1986; Askari, F. K. and McDonnell, W. M. (1996) N. Eng. J. Med. 334:316-318; Bennett, M. R. and Schwartz, S. M. (1995) Circulation 22:1981-1993; Mercola, D. and Cohen, J. S. (1995) Cancer Gene Ther. 2:47-59; Rossi, J. J. (1995) Br. Med. Bull. 51:217-225; Wagner, R. W. (1994) Nature 372:333-335). An antisense nucleic acid molecule comprises a nucleotide sequence that is complementary to the coding strand of another nucleic acid molecule (e.g., an mRNA sequence) and accordingly is capable of hydrogen bonding to the coding strand of the other nucleic acid molecule. Antisense sequences complementary to a sequence of an mRNA can be complementary to a sequence found in the coding region of the mRNA, the 5′ or 3′ untranslated region of the mRNA or a region bridging the coding region and an untranslated region (e.g., at the junction of the 5′ untranslated region and the coding region). Furthermore, an antisense nucleic acid can be complementary in sequence to a regulatory region of the gene encoding the mRNA, for instance a transcription initiation sequence or regulatory element. Preferably, an antisense nucleic acid is designed so as to be complementary to a region preceding or spanning the initiation codon on the coding strand or in the 3′ untranslated region of an mRNA.

Given the coding strand sequences of TRAF3, antisense nucleic acids of the invention can be designed according to the rules of Watson and Crick base pairing. The antisense nucleic acid molecule can be complementary to the entire coding region of the mRNA, but more preferably is an oligonucleotide which is antisense to only a portion of the coding or noncoding region of the mRNA. For example, the antisense oligonucleotide can be complementary to the region surrounding the translation start site of the mRNA. An antisense oligonucleotide can be, for example, about 5, 10, 15, 20, 25, 30, 35, 40, 45 or 50 nucleotides in length. An antisense nucleic acid of the invention can be constructed using chemical synthesis and enzymatic ligation reactions using procedures known in the art. For example, an antisense nucleic acid (e.g., an antisense oligonucleotide) can be chemically synthesized using naturally occurring nucleotides or variously modified nucleotides designed to increase the biological stability of the molecules or to increase the physical stability of the duplex formed between the antisense and sense nucleic acids, e.g., phosphorothioate derivatives and acridine substituted nucleotides can be used. Examples of modified nucleotides which can be used to generate the antisense nucleic acid include 5-fluorouracil, 5-bromouracil, 5-chlorouracil, 5-iodouracil, hypoxanthine, xantine, 4-acetylcytosine, 5-(carboxyhydroxylmethyl)uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl)uracil, (acp3)w, and 2,6-diaminopurine. To inhibit expression in cells, one or more antisense oligonucleotides can be used. Alternatively, the antisense nucleic acid can be produced biologically using an expression vector into which a nucleic acid has been subcloned in an antisense orientation (i.e., RNA transcribed from the inserted nucleic acid will be of an antisense orientation to a target nucleic acid of interest, described further in the following subsection).

In yet another embodiment, the antisense nucleic acid molecule of the invention is an α-anomeric nucleic acid molecule. An α-anomeric nucleic acid molecule forms specific double-stranded hybrids with complementary RNA in which, contrary to the usual β-units, the strands run parallel to each other (Gaultier et al. (1987) Nucleic Acids. Res. 15:6625-6641). The antisense nucleic acid molecule can also comprise a 2′-o-methylribonucleotide (Inoue et al. (1987) Nucleic Acids Res. 15:6131-6148) or a chimeric RNA-DNA analogue (Inoue et al. (1987) FEBS Lett. 215:327-330).

In another embodiment, an antisense nucleic acid of the invention is a compound that mediates RNAi. RNA interfering agents include, but are not limited to, nucleic acid molecules including RNA molecules which are homologous to the target gene or genomic sequence, “short interfering RNA” (siRNA), “short hairpin” or “small hairpin RNA” (shRNA), and small molecules which interfere with or inhibit expression of a target gene by RNA interference (RNAi). RNA interference is a post-transcriptional, targeted gene-silencing technique that uses double-stranded RNA (dsRNA) to degrade messenger RNA (mRNA) containing the same sequence as the dsRNA (Sharp, P. A. and Zamore, P. D. 287, 2431-2432 (2000); Zamore, P. D., et al. Cell 101, 25-33 (2000). Tuschl, T. et al Genes Dev. 13, 3191-3197 (1999)). The process occurs when an endogenous ribonuclease cleaves the longer dsRNA into shorter, 21- or 22-nucleotide-long RNAs, termed small interfering RNAs or siRNAs. The smaller RNA segments then mediate the degradation of the target mRNA. Kits for synthesis of RNAi are commercially available from, e.g. New England Biolabs and Ambion. In one embodiment one or more of the chemistries described above for use in antisense RNA can be employed.

In still another embodiment, an antisense nucleic acid of the invention is a ribozyme. Ribozymes are catalytic RNA molecules with ribonuclease activity which are capable of cleaving a single-stranded nucleic acid, such as an mRNA, to which they have a complementary region. Thus, ribozymes (e.g., hammerhead ribozymes (described in Haselhoff and Gerlach, 1988, Nature 334:585-591) can be used to catalytically cleave KRC mRNA transcripts to thereby inhibit translation of KRC mRNA. A ribozyme having specificity for a TRAF3-encoding nucleic acid can be designed based upon the nucleotide sequence of TRAF3. For example, a derivative of a Tetrahymena L-19 IVS RNA can be constructed in which the nucleotide sequence of the active site is complementary to the nucleotide sequence to be cleaved in a TRAF3-encoding mRNA. See, e.g., Cech et al. U.S. Pat. No. 4,987,071; and Cech et al., U.S. Pat. No. 5,116,742. Alternatively, TRAF3 mRNA can be used to select a catalytic RNA having a specific ribonuclease activity from a pool of RNA molecules. See, e.g., Bartel, D. and Szostak, J. W., 1993, Science 261:1411-1418.

Alternatively, gene expression can be inhibited by targeting nucleotide sequences complementary to the regulatory region of TRAF3 (e.g., the KRC promoter and/or enhancers) to form triple helical structures that prevent transcription of the TRAF3 gene in target cells. See generally, Helene, C., 1991, Anticancer Drug Des. 6(6):569-84; Helene, C. et al., 1992, Ann. N.Y. Acad. Sci. 660:27-36; and Maher, L. J., 1992, Bioassays 14(12):807-15.

In yet another embodiment, the TRAF3 nucleic acid molecules of the present invention can be modified at the base moiety, sugar moiety or phosphate backbone to improve, e.g., the stability, hybridization, or solubility of the molecule. For example, the deoxyribose phosphate backbone of the nucleic acid molecules can be modified to generate peptide nucleic acids (see Hyrup B. et al., 1996, Bioorganic & Medicinal Chemistry 4 (I): 5-23). As used herein, the terms “peptide nucleic acids” or “PNAs” refer to nucleic acid mimics, e.g., DNA mimics, in which the deoxyribose phosphate backbone is replaced by a pseudopeptide backbone and only the four natural nucleobases are retained. The neutral backbone of PNAs has been shown to allow for specific hybridization to DNA and RNA under conditions of low ionic strength. The synthesis of PNA oligomers can be performed using standard solid phase peptide synthesis protocols as described in Hyrup B. et al., 1996, supra; Perry-O'Keefe et al., 1996, Proc. Natl. Acad. Sci. USA 93: 14670-675.

PNAs of TRAF3 nucleic acid molecules can be used in therapeutic and diagnostic applications. For example, PNAs can be used as antisense or antigene agents for sequence-specific modulation of gene expression by, for example, inducing transcription or translation arrest or inhibiting replication. PNAs of TRAF3 nucleic acid molecules can also be used in the analysis of single base pair mutations in a gene, (e.g., by PNA-directed PCR clamping); as ‘artificial restriction enzymes’ when used in combination with other enzymes, (e.g., S1 nucleases (Hyrup B., 1996, supra)); or as probes or primers for DNA sequencing or hybridization (Hyrup B. et al., 1996, supra; Perry-O'Keefe supra).

In another embodiment, PNAs of TRAF3 can be modified, (e.g., to enhance their stability or cellular uptake), by attaching lipophilic or other helper groups to PNA, by the formation of PNA-DNA chimeras, or by the use of liposomes or other techniques of drug delivery known in the art. For example, PNA-DNA chimeras of KRC nucleic acid molecules can be generated which may combine the advantageous properties of PNA and DNA. Such chimeras allow DNA recognition enzymes, (e.g., RNAse H and DNA polymerases), to interact with the DNA portion while the PNA portion would provide high binding affinity and specificity. PNA-DNA chimeras can be linked using linkers of appropriate lengths selected in terms of base stacking, number of bonds between the nucleobases, and orientation (Hyrup B., 1996, supra). The synthesis of PNA-DNA chimeras can be performed as described in Hyrup B., 1996, supra and Finn P. J. et al., 1996, Nucleic Acids Res. 24 (17): 3357-63. For example, a DNA chain can be synthesized on a solid support using standard phosphoramidite coupling chemistry and modified nucleoside analogs, e.g., 5′-(4-methoxytrityl)amino-5′-deoxy-thymidine phosphoramidite, can be used as a between the PNA and the 5′ end of DNA (Mag, M. et al., 1989, Nucleic Acid Res. 17: 5973-88). PNA monomers are then coupled in a stepwise manner to produce a chimeric molecule with a 5′ PNA segment and a 3′ DNA segment (Finn P. J. et al., 1996, supra). Alternatively, chimeric molecules can be synthesized with a 5′ DNA segment and a 3′ PNA segment (Peterser, K H. et al., 1975, Bioorganic Med. Chem. Lett. 5: 1119-11124).

In other embodiments, the oligonucleotide may include other appended groups such as peptides (e.g., for targeting host cell receptors in vivo), or agents facilitating transport across the cell membrane (see, e.g., Letsinger et al., 1989, Proc. Natl. Acari Sci. US. 86:6553-6556; Lemaitre et al., 1987, Proc. Natl. Acad. Sci. USA 84:648-652; PCT Publication No. WO88/09810) or the blood-brain barrier (see, e.g., PCT Publication No. WO89/10134). In addition, oligonucleotides can be modified with hybridization-triggered cleavage agents (See, e.g., Krol et al., 1988, Bio-Techniques 6:958-976) or intercalating agents. (See, e.g., Zon, 1988, Pharm. Res. 5:539-549). To this end, the oligonucleotide may be conjugated to another molecule, (e.g., a peptide, hybridization triggered cross-linking agent, transport agent, or hybridization-triggered cleavage agent).

Antisense polynucleotides may be produced from a heterologous expression cassette in a transfectant cell or transgenic cell. Alternatively, the antisense polynucleotides may comprise soluble oligonucleotides that are administered to the external milieu, either in the culture medium in vitro or in the circulatory system or in interstitial fluid in vivo. Soluble antisense polynucleotides present in the external milieu have been shown to gain access to the cytoplasm and inhibit translation of specific mRNA species.

In another embodiment, an inhibitory compound of the invention is a peptidic compound derived from the TRAF3 amino acid sequence. In particular, the inhibitory compound comprises a portion of TRAF3 (or a mimetic thereof) that mediates interaction of TRAF3 with a target molecule such that contact of TRAF3 with this peptidic compound competitively inhibits the interaction of TRAF3 with the target molecule.

The peptidic compounds of the invention can be made intracellularly in immune cells by introducing into the immune cells an expression vector encoding the peptide. Such expression vectors can be made by standard techniques, using, for example, oligonucleotides that encode the amino acid sequences of TRAF3. The peptide can be expressed in intracellularly as a fusion with another protein or peptide (e.g., a GST fusion). Alternative to recombinant synthesis of the peptides in the cells, the peptides can be made by chemical synthesis using standard peptide synthesis techniques. Synthesized peptides can then be introduced into cells by a variety of means known in the art for introducing peptides into cells (e.g., liposome and the like).

In another embodiment, an inhibitory agent is an antagonistic binding molecule, i.e., a binding molecule that inhibits the activity of TRAF3. The production of such binding molecules is described above.

Other inhibitory agents that can be used to specifically inhibit the activity of a TRAF3 protein are chemical compounds that directly inhibit TRAF3 activity or inhibit the interaction between TRAF3 and target molecules. Such compounds can be identified using screening assays that select for such compounds, as described above.

### 6. Kits of the Invention

The present invention provides kits for use of the methods of the present invention. In one embodiment of the invention, the kit, comprises a detectable agent that specifically recognizes TRAF3, TRAF2, and/or p53, instructions for use. The kit may optionally contain reagents for isolating a sample from a tumor cell.

### EXAMPLES

The present invention is further illustrated by the following examples which should not be construed as limiting in any way.

### Example 1

### TRAF3 Controls LTβR Signaling in Tumor Cells

### Introduction

The object of this study was to determine how these TRAFs, particularly TRAF3, interacts with different receptors in the TNFR superfamily, particularly LTβR, to selectively activate different NFκB pathways. The study also examined if and how TRAFs induce the switch from initial NFκB1- to late NFκB2-dependent gene transcription through a single receptor-ligand pair.

The results show that LTβR recruits both TRAF2 and TRAF3 into receptor complexes upon activation. The results determined that an excess of cytoplasmic TRAF3 inhibits NFκB1 signals from LTβR, in part, by displacing TRAF2 and IKKα from receptor-complexes. It was also determined that TRAF3 inhibits the basal repression of NFκB2 by suppression of NIK mediated p100 processing, and showed that the loss of TRAF3 relieved this suppression in a receptor-independent manner Finally, the results provide evidence that NFκB2 activation is sustained through a positive-autoregulatory loop that involves NFκB2-dependent transcription and resynthesis of RelB and p100.

### Materials and Methods

The following materials and methods were used to perform the examples described herein:

### Cell Lines and Antibodies

DLD-1 and WiDr adenocarcinoma cell lines were obtained from ATCC (Manassas, Va.), and cultured in MEM Earle's medium supplemented with 10% FBS. Antibodies against LTβR (N-15), TRAF3 (H-20 and H-122), TRAF2 (H-249), and NFκB2 (C-5) were obtained from Santa Cruz Biotechnology (Santa Cruz, Calif.). Antibodies against phosphorylated IκBα (5A5), phosphorylated RelA, and NIK were purchased from Cell Signaling Technologies (Beverly, Mass.).

### Cellular Activation

LTβR was activated using 100 ng/ml of a humanized bi-specific, activating antibody (BS-1) developed at Biogen Idec, Inc. TNFα was used at a concentration of 20 ng/ml. During stimulation, cells were kept in a 37° C. incubator for indicated times.

### Immunoprecipitation and Western Blots

Cells were treated for 10 minutes at 37° C. with media or activating antibody (BS-1) and washed twice in ice-cold PBS with protease and phosphatase inhibitors, followed by lysis by scraping in ice-cold lysis buffer [50 mM PIPES pH 6.8, 100 mM KCl, 2 mM MgCl₂, 1 mM EGTA, 0.2% NP-40, and 10% Glycerol, supplemented with Complete EDTA-free protease inhibitor cocktail (Roche, Indianapolis, Ind.), 10 mM Sodium Fluoride, and 100 μM Sodium Orthovanadate]. Lysates were centrifuged at 14000 RPM for 30 minutes at 4° C., and supernatants were pre-cleared with normal goat IgG-agarose beads (Sigma, St. Louis, Mo.) by incubating on a rotator at 4° C. After centrifugation at 14000 RPM for an additional 15 minutes at 4° C., the supernatant was incubated with goat anti-human IgG-agarose beads (Sigma) for 1 hour at 4° C. on a rotator. The beads were added to Handee mini spin-columns (Pierce, Rockford, Ill.), washed five times in lysis buffer and eluted in Criterion XT loading buffer supplemented with XT-Reducing agent (Bio-Rad, Hercules, Calif.) and protease and phosphatase inhibitors. Samples were run in Criterion XT precast gels (Bio-Rad) and blotted onto nitrocellulose membranes. HRP-conjugated secondary antibodies (anti-rabbit and anti-mouse) were from GE Healthcare/Amersham (Piscataway, N.J.), and HRP-conjugated anti-goat TrueBlot antibodies were from eBioscience (San Diego, Calif.). Blots were developed using SuperSignal chemiluminescent detection substrates (Pierce), and detected on Biomax X-ray films (Kodak, Rochester, N.Y.) and/or directly on a Kodak ImageStation 2000R luminescence detector.

### RNA Interference

siGENOME SMARTPool siRNA pools (Dharmacon, Lafayette, Colo.) against selected targets were transfected at concentrations of 100-200 nM using Lipofectamine 2000 (Invitrogen, Carlsbad, Calif.). 48-72 hours after transfection, cells were treated and lysed in Criterion XT buffer (Bio-Rad) for western blots or QIAzol (Qiagen, Valencia, Calif.) for RNA isolation.

Experiments were performed using pools consisting of four individual siRNAs for each target, except for the non-silencing control (NS), for which a single siRNA was used. Sequences are written in (sense|antisense) format, except for NS, for which only the sense strand is written.

NIK (M-003580-03):
1. UCUCAAAGCUCGCGGGACAUU | UGUCCCGCGAGCUUUGAGAUU
2. GGGAAAGCGUCGCAGCAAAUU | UUUGCUGCGACGCUUUCCCUU
3. CGCCAAAUCAAGCCAAUUAUU | UAAUUGGCUUGAUUUGGCGUU
4. UCACCAAGCCUCUGAAGGAUU | UCCUUCAGAGGCUUGGUGAUU
LTβR (M-008023-01):
1. CCACAUGUGCCGAGAAUUCUU | GAAUUCUCGGCACAUGUGGUU
2. CCAAGGAACCAAUUUAUCAUU | UGAUAAAUUGGUUCCUUGGUU
3. CAACAUCUACAUCUACAAUUU | AUUGUAGAUGUAGAUGUUGUU
4. GAACCAAUUUAUCACCCAUUU | AUGGGUGAUAAAUUGGUUCUU
TRAF3 (M-005252-00):
1. GAUAAGGUGUUUAAGGAUAUU | UAUCCUUAAACACCUUAUCUU
2. GCAAGUGGCUCGGAACACAUU | UGUGUUCCGAGCCACUUGCUU
3. GACAUCUGCUGGUGCAUUUUU | AAAUGCACCAGCAGAUGUCUU
4. GGCCCAAACUGUUCUAGAAUU | UUCUAGAACAGUUUGGGCCUU
IKK1 (M-003473-01):
1. CAAAGAAGCUGACAAUACUUU | AGUAUUGUCAGCUUCUUUGUU
2. CCAGAUACUUUCUUUACUAUU | UAGUAAAGAAAGUAUCUGGUU
3. GAAGUUCGGUUUAGUAGCCUU | GGCUACUAAACCGAACUUCUU
4. AAAUAUGGCAUCUCCUUAAUU | UUAAGGAGAUGCCAUAUUUUU
TRAF2 (M-005198-00):
1. GGAGCAUUGGCCUCAAGGAUU | UCCUUGAGGCCAAUGCUCCUU
2. GCAGGUACGGCUACAAGAUUU | AUCUUGUAGCCGUACCUGCUU
3. CGGUAGAGGGUGAGAAACAUU | UGUUUCUCACCCUCUACCGUU
4. GAAGAAGGCAUUUCUAUUUUU | AAAUAGAAAUGCCUUCUUCUU
NS, non-silencing control (D-001210-01):
1. UAGCGACCUAAACACAUCAA

Real-Time qPCR and Transcript Profiling

RNA was isolated by extraction in QIAzol, followed by RNeasy columns (Qiagen). For real-time RT-PCR, primer and fluorescent probe sets for selected genes and a GAPDH control were obtained from Applied Biosystems (Foster City, Calif.) and used in RT-PCR reaction using Quanti-tect Probe RT-PCR kit (Qiagen) and a MJ Research thermal cycler with an optical detector (Bio-Rad). Transcript-profiling was then performed.

p53Staining

Formalin fixed paraffin embedded sections of tumor from the orthotopic CRC study were prepared for immunohistochemistry using standard procedures. Slides were deparaffinized, rehydrated and blocked in 5% horse serum. The primary staining antibody for p53 detection was DO7 (Dako Cytomation, #M 7001) used at a final concentration of 1:25. Secondary antibody detection was performed using the Vectastain Elite ABC kit for Mouse IgG (Vector laboratories cat# PK-6102) in which a biotinylated anti mouse secondary antibody is applied, followed by ABC enhancement and peroxidase substrate for detection. Slides were evaluated by light microscopy.

### Colorectal Cancer Arrays

Human colorectal cancer tissues were purchased in a microarray slide from Imgenex (IMH-306). These slides were prepared for p53 immunohistochemistry by the method described above. Tissues in the array were read by light microscopy and scored by the investigator for negative, heterogeneous or high p53 staining

### Example 1.1

### Identification of Adenocarcinoma Cells Incapable of Responding to LTβR Stimulation

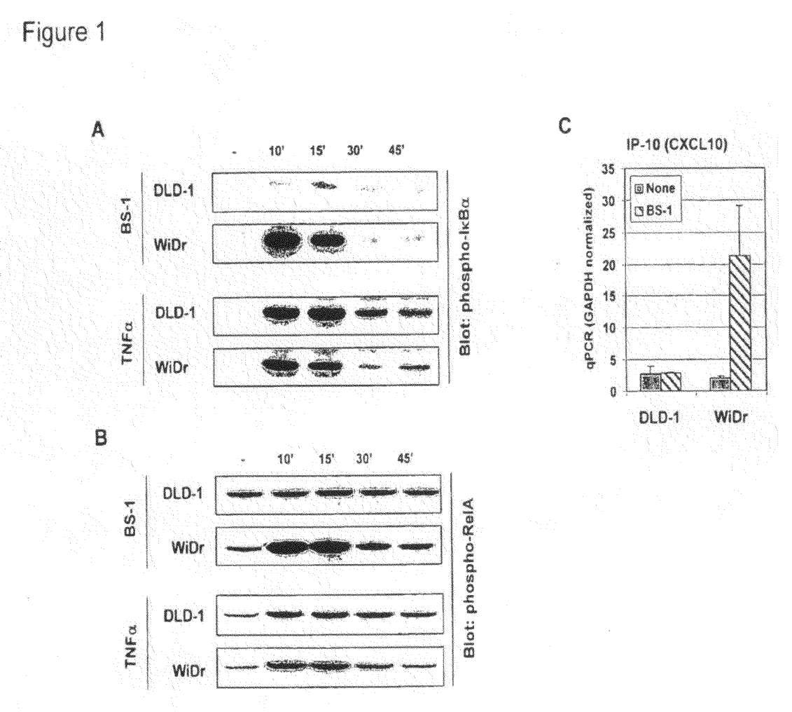

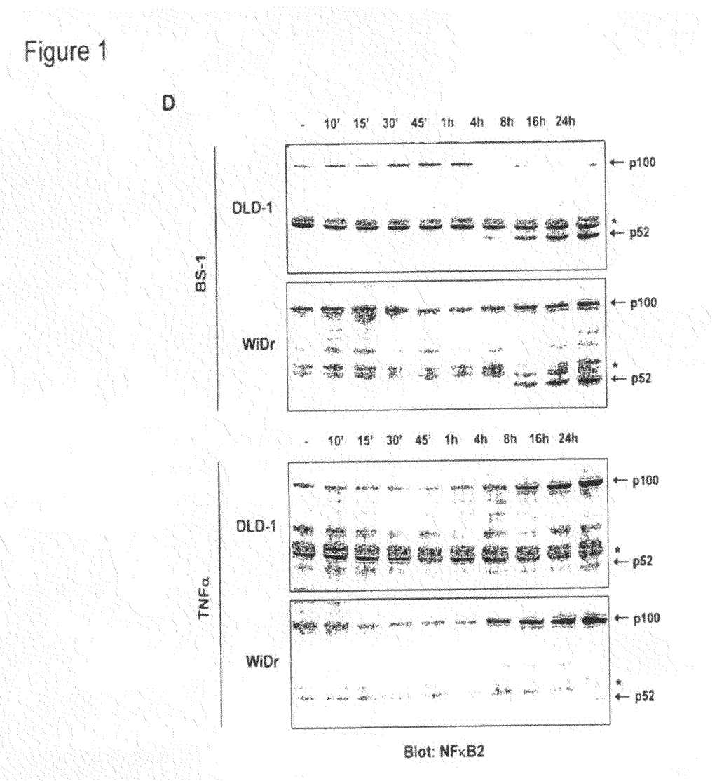

The following describes the identification of DLD-1 adenocarcinoma cells as being incapable of activating early NFκB1 in response to LTβR activation. Previous studies have shown that LTβR ligation by agonist antibodies results in the death of some adenocarcinoma cell-lines (Browning, Miatkowski et al. 1996). In previous screens, it was observed that while the WiDr cell-line was sensitive to this death, i.e., LTβR agonist antibody death, the DLD-1 cell-line was not. In looking for possible differences in LTβR-mediated signaling among these cell-lines that might account for this variation in response to the LTβR agonist antibodies, it was determined that while WiDr cells activated the canonical NFκB1 pathway, as evidenced by phosphorylation of IκBα and RelA, these phosphorylation events did not occur in DLD-1 cells (see FIGS. 1A and 1B, top half). Additionally, while WiDr cells activated TP-10 gene transcription in response to the agonist antibodies, DLD-1 cells were unable to activate this gene, which is a target of NFκB1-dependent transcription (FIG. 1C). However, LTβR activation-induced processing of p100 NFκB2 to p52, an event indicative of NFκB2 activation, was apparent and similar in both cell-lines (FIG. 1D), indicating that DLD-1 cells were specific in their inability to activate NFκB1. Moreover, the activation of NFκB1 by a different stimulus (TNFα) was identical in both cell-lines (FIGS. 1A and 1B, bottom half), which indicated that the inability to activate NFκB1 was LTβR-specific, and ruled out deficiencies in the general NFκB1 signaling machinery. Since changes in LTβR expression between the cell lines could account for the differences in signaling ability, we looked for, but did not find, any mutations in the coding region of LTβR (data not shown). Although DLD-1 cells expressed slightly lower levels of the receptor on their cell surface than WiDr cells, the difference was minimal, and therefore could not, by itself, explain the inability of these cells to signal through NFκB1. These results showed that certain cancer cell lines, such as DLD-1 cells, had a LTβR-specific defect in NFκB1 activation.

### Example 1.2

### NFκB1 Activation by LTβR Correlates with Decreased Levels of TRAF3

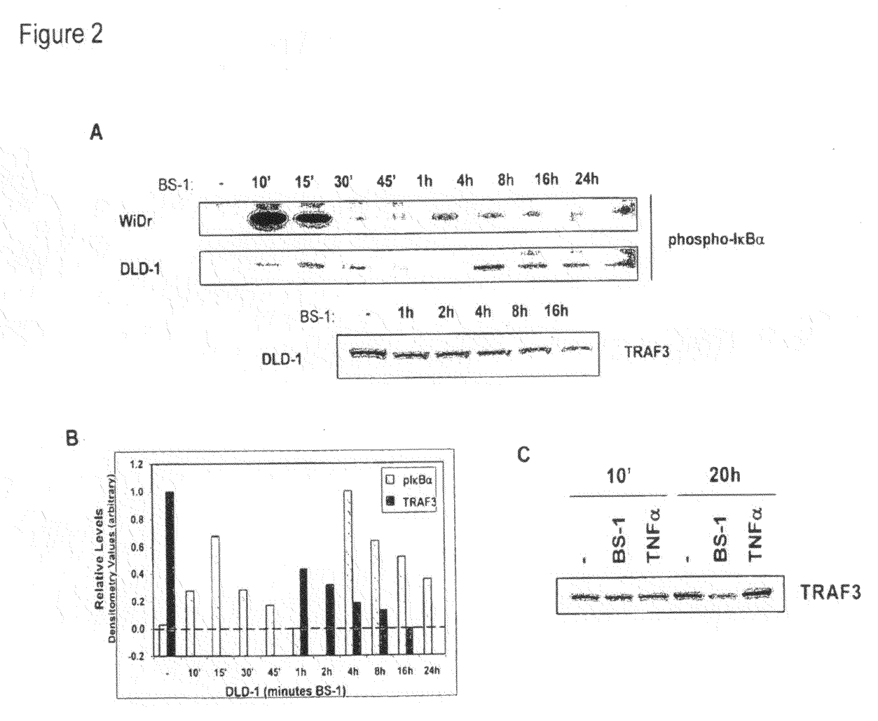

Consistent with reports that NFκB1 activation is oscillatory with temporally decreasing amplitudes (Hoffmann, Levchenko et al. 2002; Nelson, Ihekwaba at al. 2004); it was shown that activation of LTβR resulted in oscillatory IκBα phosphorylation in WiDr cells. A rapid and transient IκBα phosphorylation was detected at around 10 min; and another wave of phosphorylation, albeit of a much lower magnitude, was detectable at around 4 h (FIG. 2A). It was determined that while DLD-1 cells did not activate the first wave of IκBα phosphorylation at 10 min, the second wave of phosphorylation at 4 h was detectable and was similar in magnitude to that observed in WiDr cells (FIG. 2A). These results suggested that DLD-1 cells were able to eventually regain their NFκB1 signaling potential, and suggested that other LTβR activation-induced changes might account for this restoration. In testing for LTβR and known receptor-associated molecules involved in signaling, it was determined that activation of the second wave of IκBα phosphorylation correlated with a reduction in the levels of TRAF3 in DLD-1 cells (FIGS. 2A and 2B). Since TRAF3 has been implicated to be a negative regulator of CD40 signaling (Hostager and Bishop 1999; He, Grammer et al. 2004), and consistent with results from LTβR (FIG. 2C), as well as with reports that CD40 and BAFF also downregulate TRAF3 during signaling (Moore and Bishop 2005; Morrison, Reiley et al. 2005), it was suggestive that TRAF3 levels might be different in these cells, and thus the putative inhibitor of early NFκB1 signaling in DLD-1 cells.

### Example 1.3

### DLD-1 and WiDr Cells have Different Levels of Cytoplasmic TRAF3, and this Correlates with the Formation of different LTβR-Associated Signaling Complexes

The following study shows that two different cancer cell lines, i.e., DLD-1 and WiDr cells, have different levels of cytoplasmic TRAF3, which correlates with the formation of different LTβA-associated signaling complexes.

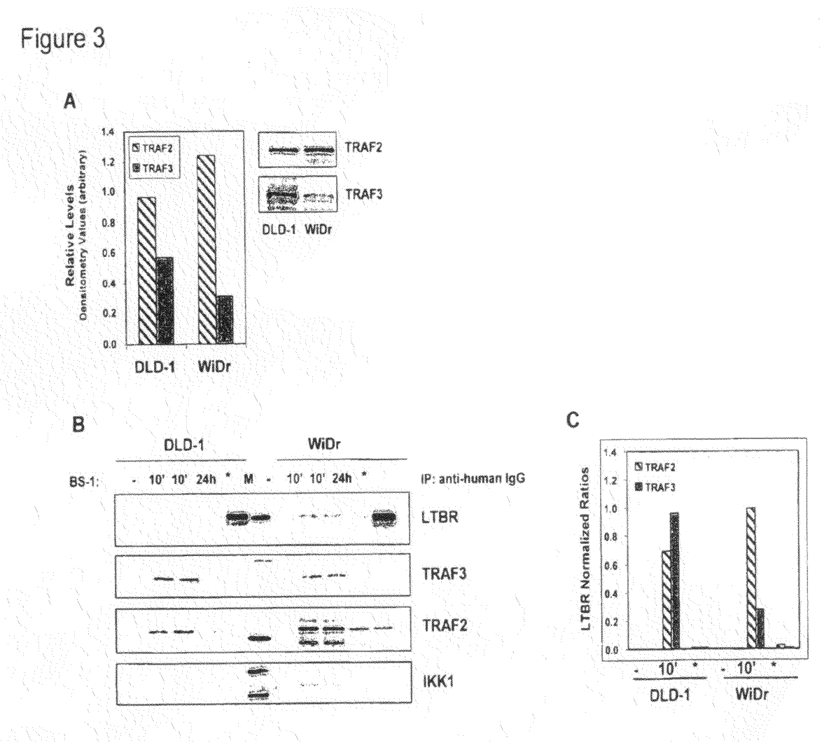

Following suggesting that different TRAF3 levels were different in the various cancer cells, it was determined that higher cytoplasmic levels of TRAF3 in DLD-1 cells compared to WIDr cells (FIG. 3A).

Subsequently, studies were performed to determine whether this comparatively high level of TRAF3 observed in DLD-1 cells resulted in differences in the formation of receptor-associated signaling complexes. The cells were incubated with agonist LTβR antibodies to allow formation of the complexes, washed and lysed the cells, and immunoprecipitated the agonist antibodies bound to LTβR. Western blot of the immunoprecipitates from DLD-1 cells revealed a higher ratio of TRAF3 to LTβR compared to the immunoprecipitates from WiDr cells (FIGS. 3B and 3C). To rule out non-specific binding and activation, LTβR was immunoprecipitated from unstimulated cells. This immunoprecipitation was unable to find significant association of TRAFs associated with the receptor (FIGS. 3B and 3C, * lanes). In addition to the higher levels of TRAF3, slightly lower levels of TRAF2 were also found, and almost undetectable levels of IKKα in DLD-1 immunoprecipitates (FIG. 3B) at 10 min. These results suggested strongly that increased expression of TRAF3 might alter the formation of LTβR activation-induced receptor-complexes, thereby providing a mechanism for TRAF3-induced suppression of NFκB1 in these cells.

### Example 1.4

### Restoration of LTβR Activation in DLD-1 Cells

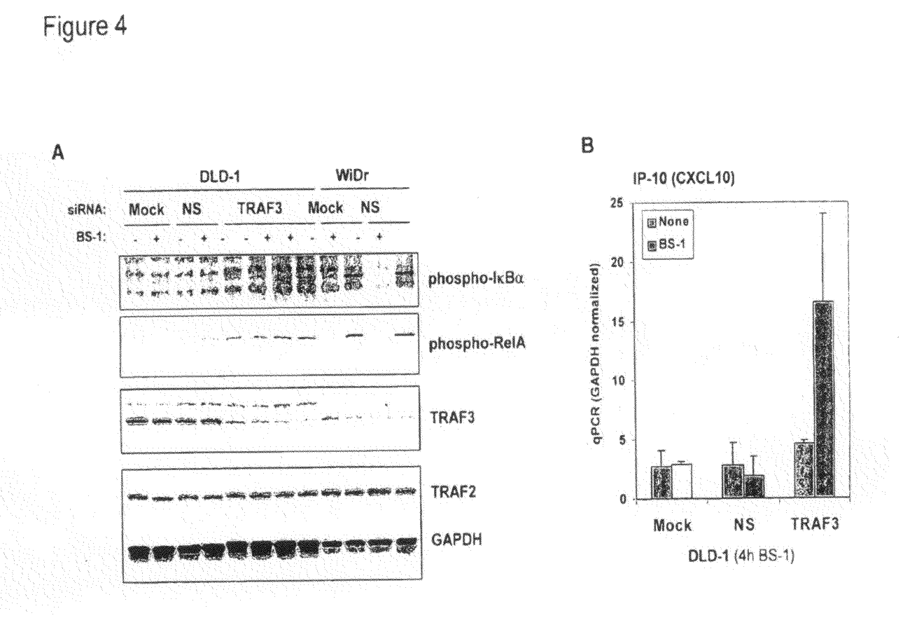

The following study demonstrates that inhibition of TRAF3, using RNAi mediated downregulation of TRAF3, restores LTβR activation-induced NFκB1 signaling in DLD-1 cells. The following experiments were performed to test whether the higher levels of TRAF3 in the cytoplasm and LTβR-complexes of DLD-1 cells accounted for the inability of these cells to signal through NFκB1 in response to LTβR stimulation. DLD-1 cells were transfected with siRNA targeted against TRAF3. After culturing the cells for 48 h to allow silencing of endogenous TRAF3, the cells were stimulated with agonist LTβR antibodies for 10 min. It was determined that RNAi-mediated knockdown in DLD-1 cells was able to reduce TRAF3 levels comparable to levels in WiDr cells (FIG. 4A, ‘TRAF3’ lanes). As a control, cells were transfected with a non-silencing control siRNA, and this transfection did not reduce TRAF3 levels (FIG. 4A, ‘NS’ lanes). More importantly, it was determined that TRAF3 siRNA, but not non-silencing siRNA, restored the ability of DLD-1 cells to phosphorylate IκBα and RelA upon LTbR activation (FIG. 4A). The NFκB1-dependent transcriptional activation of IP-10 was also restored in TRAF3 siRNA transfected DLD-1 cells, and served as a marker for the functional consequence of NFκB1 activation (FIG. 4B).

### Example 1.5

### High Level of TRAF3 Alters the Composition of LTβR-Associated Signaling Complexes, Reducing the Levels of TRAF2 and IKKα Bound to the Receptor

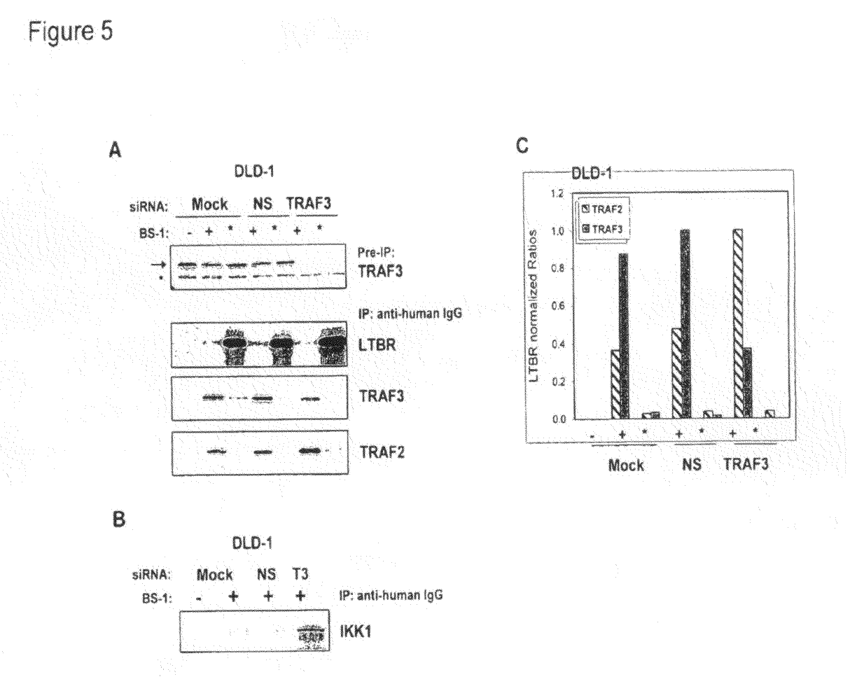

Since altered composition of LTβR-associated signaling complexes between the two cell lines (see FIG. 3C) was observed, the following was performed to determine whether cytoplasmic TRAF3 level influences the composition of LTβR-associated signaling complexes. Using siRNA mediated knockdown of TRAF3 in DLD-1 cells, agonist-antibody-LTβR complexes were immunoprecipitated from stimulated cells. The experiment determined that the immunoprecipitates in TRAF3-knockdown cells had a reduced ratio of TRAF3 to receptor (FIG. 5A). In addition, increases in levels of TRAF2 and IKKα bound to the receptor in these cells were observed (FIGS. 5A and 5B). TRAF3-knockdown thus changed the composition of LTβ-associated signaling complexes in DLD-1 cells compared to control-knockdown cells (FIGS. 5B and 5C), making these complexes more similar to those in WiDr cells (compare with FIG. 3). These results provide evidence that TRAF3 inhibits LTβR-induced NFκB1 signals, in part, by limiting TRAF2 and IKKαrecruited to the receptor.

### Example 1.6

### TRAF3 Inhibits LTβR-Independent Processing of NFκB1 p100

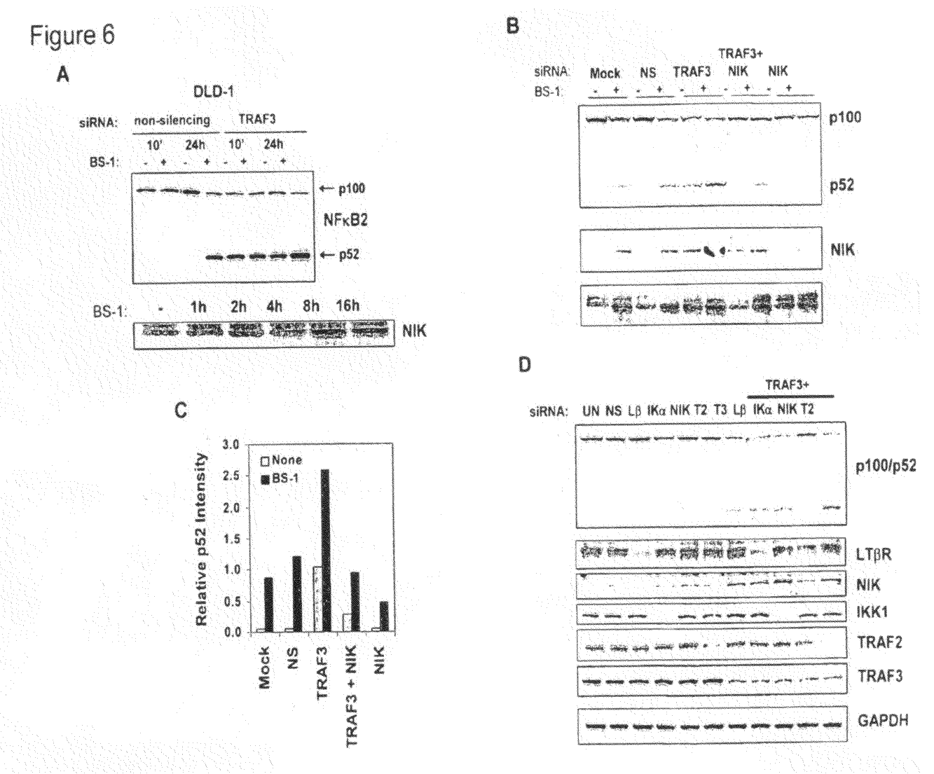

It has been reported that NIK mediated processing of NFκB2 p100 into p52 can be suppressed by the overexpression of TRAF3, in a manner that involves TRAF3-directed proteasomal degradation of NIK (Liao, Zhang et al. 2004). Since siRNA targeted against TRAF3 in DLD-1 cells had been used (as described above), studies were performed to determine whether the converse of this was also true: that is, whether the knockdown of endogenous levels of TRAF3 resulted in stabilization of NIK. It was determined that TRAF3 siRNA-transfected DLD-1 cells had reduced TRAF3 protein levels and a coordinate increase in NIK protein levels (FIG. 6B). Surprisingly, however, it was determined that these cells also processed NFκB2 p100 into p52 in a stimulus-independent fashion (FIG. 6A, right half, ‘−’ lanes). Confirming the previous data from overexpression studies (Liao, Zhang et al. 2004), this processing was dependent on NIK, as combined-knockdown of TRAF3 and NIK reduced the levels of processed p100 (FIG. 6B). However, this processing was not dependent on LTβR (FIG. 6D, ‘Lβ’ lanes), as siRNA-mediated silencing of both LTβR and TRAF3 did not change the levels of p52 processed than when TRAF3 was silenced alone. Surprisingly, this processing was also independent of IKKα (FIG. 6D, ‘IKα’ lanes). These results show that removing the TRAF3-mediated repression of NIK can activate the alternative NFκB pathway, and that such activation is independent of LTβR or IKKα.

### Example 1.7

### The Alternative NFβB Pathway is Regulated by a Positive-Autoregulatory Loop

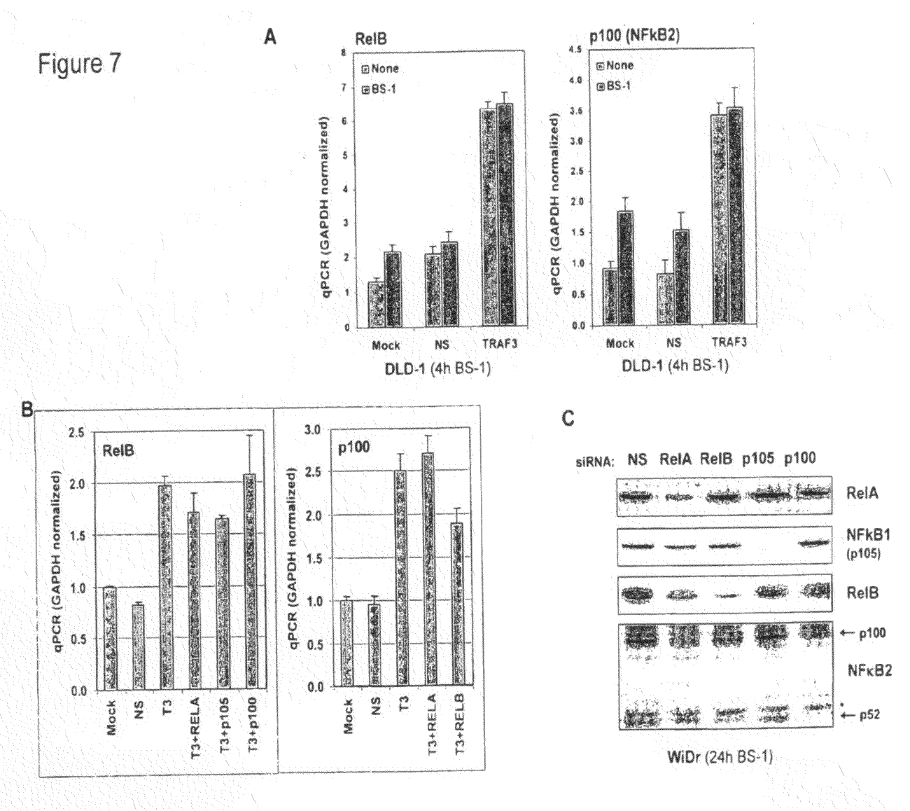

The above experiments with TRAF3 knockdown provided another interesting, but unexpected, insight into the mechanism of NFκB2 regulation. As described above, for the restoration of NFκB1 activation in DLD-1 cells, real-time qPCR was performed on RNA extracted from cells transfected with TRAF3 siRNA for RelB, and p100, both of which have been previously described as NFκB1 target genes. Surprisingly, it was determined that DLD-1 cells transfected with TRAF3-siRNA, but not control siRNA, had elevated RelB and p100 transcripts in the unstimulated state (FIG. 7A, ‘TRAF3’samples, solid bars). Since NFκB1 activation was stimulus-dependent in these cells (see FIG. 4, ‘TRAF3’ samples), but NFκB2 activation was not (see FIG. 6A), additional experiments were performed to ascertain whether the increase in RelB and p100 that was observed was instead NFκB2 dependent, and thus transfected the cells with siRNA against both TRAF3 and individual transcriptional components of NFκB1 and NFκB2 pathways. It was determined that while TRAF3-knockdown mediated increase in basal p100 transcripts was not suppressed, or suppressed at a very low level, by knockdown of RelA or p105, they were suppressed quite significantly by knockdown of RelB (FIG. 7B). However, the basal increase in RelB transcripts in TRAF3-knockdown cells was not suppressed by knockdown of p100, RelA or p105 (FIG. 7B). To confirm these results, siRNAs were singly transfected against components of the NFκB1 and NFκB2 pathways in WiDr cells and upon stimulation to activate LTβR, found, using western blots, that RelB and p100 knockdown cross-suppressed each other (FIG. 7C), although the suppression of RelB from p100-knockdown was very slight compared to the suppression of p100 from RelB-knockdown. These results suggest that NFκB2 is positively autoregulated.

### Discussion

The studies described above in Examples 1.1 to 1.7 that TRAF3 is a critical switch in LTβR activation and negatively regulates both NFκB pathways: by a stimulus- and receptor-dependent inhibition of NFκB1, and by stimulus-independent inhibition of NFκB2. Furthermore, these examples also show that TRAF3 is an inhibitor of the positive-autoregulatory NFκB2 loop, and the loss of TRAF3 releases this inhibition, allowing the sustained synthesis and activation of NFκB2 components.

LTβR-stimulation results in the rapid activation of IκBot and RelA phosphorylation, usually within the first ten minutes of stimulation, followed by the processing of NFκB2 p100 to p52, detectable starting at around 4 hours of stimulation (our results, and (Dejardin, Droin et al. 2002; Kim, Nedospasov et al. 2005)). Similar NFκB1 and NFκB2 activation events are observed in cases of CD40, BAFF-R, and TWEAK signaling (Kayagaki, Yan et al. 2002; Saitoh, Nakayama et al. 2003; Grech, Amesbury et al. 2004; Ramakrishnan, Wang et al. 2004; Zarnegar, He et al. 2004), all of which are in the TNFR-family subset that can activate both NFκB pathways. However, the culmination of signaling studies from these receptors indicate that NFκB1 and NFκB2 are activated sequentially, and there are no reported instances of exclusive NFκB2 activation without first the activation of NFκB1. The results described above from LTβR stimulation in DLD-1 cells provide the first suggestion that the activation of NFκB1 is not a prerequisite to the activation of NFκB2, and allow the dissection of the LTβR-specific contribution to discrete events in signaling via the two arms.

Furthermore, the above examples show that TRAF3 is a molecular switch that inhibits the LTβR-dependent activation of NFκB1, and subsequent apoptosis of cancer cells. This observation is consistent with the reported role of TRAF3 as an inhibitor in CD40 and BAFF-R signaling (Hostager and Bishop 1999; Xu and Shu 2002). The mechanism of TRAF3-mediated inhibition of NFκB1 signaling is not known, but a reasonable possibility, which is in keeping with the above results, and of others (He, Grammer et al. 2004; Xie, Hostager et al. 2004), is that excess TRAF3 prevents recruitment of components in receptor complexes that are necessary for NFκB1 activation, such as TRAF2 and IKKα. The role for TRAF2 in this activation is more apparent, as TRAF2 is a required component of CD40 and LTβR-dependent NFκB1 signaling (Hostager and Bishop 1999; Grech, Amesbury et al. 2004; Kim, Nedospasov et al. 2005), and regulates the degradation of itself and TRAF3 (Brown, Hostager et al. 2002; Moore and Bishop 2005). The role for IKKα in NFκB1 activation, however, is a little less clear. Although IKKα is a part of the IKK-complex that is involved in IκBα and RelA phosphorylation, IKKα involvement has been reported to be more important for NFκB2 activation (Hayden and Ghosh 2004), and IKKβ and NEMO/IKKγ instead have been shown to be more crucial mediators of NFκB1 activation, since IKKαknockout mice retain the ability to phosphorylate IκBα (Hu, Baud et al. 1999; Li, Estepa et al. 2000). Despite these results, other studies of NFκB1 signaling using IKKα- and IKKβ-knockout and knockdown cells reveal a more active role for IKKα in RelA phosphorylation (Sizemore, Lerner et al. 2002; Sakurai, Suzuki et al. 2003). Moreover, LTβR activated nuclear lysates from IKKαknockout cells fail to form any RelA/p50 DNA-binding activity, while IKKβ knockout cells display diminished, but clearly detectable, DNA-binding activity (Derudder, Dejardin et al. 2003). The above-mentioned results suggest an involvement of IKKα in LTβR signaling complexes for the activation of the classical arm, since IKKα is a part of classical NFκB signal-competent receptor-complexes (FIGS. 3B and 5B), but not of non-signaling complexes. Moreover, these results also suggest that the IKKα is not necessary for basal activation of the alternative arm induced by the loss of TRAF3 (FIG. 6D). This is a surprising finding given the conventional role of IKKα in p100 processing, however, it is consistent with previous results that while the elimination of IKKαallows residual p100 to p52 processing during LTβR stimulation, the elimination of both IKKα and IKKβ eliminates this processing (Dejardin, Droin et al. 2002). Further studies will need to be done to further define the role of IKKα in p100 processing.

Unlike TRAF3's role in inhibiting stimulus-dependent NFκB1 activation, its mode of NFκB2 inhibition is stimulus-independent. TRAF3 has been shown to be a negative regulator of NFκB2 activation (Hauer, Puschner et al. 2005). This occurs most likely by TRAF3's ability to destabilize NIK, a kinase that activates IKKα-dependent NFκB2 p100 processing into p52 (Liao, Zhang et al. 2004). Co-overexpression of NIK with TRAF3 in 293 cells results in NM degradation (Liao, Zhang et al. 2004), and this is consistent with another report that infers, albeit in an overexpression system, the inhibitory role of TRAF3 in NFκB2 activation (Hauer, Puschner et al. 2005). Also consistent with TRAF3's role as an inhibitor of NIK is the observation (ours and (Liao, Zhang et al. 2004; Moore and Bishop 2005)) that signaling through receptors that activate NFκB2, such as LTβR, but not by TNFα (which actually increases the levels of TRAF3, see FIG. 2C), results in the degradation of TRAF3. This explains the ability of these receptors to selectively activate NFκB2, and suggests that the activation of NFκB2 is minimally dependent on reduction of TRAF3 levels to relieve its destabilization of NIK. This is consistent with the above-mentioned results of TRAF3-knockdown in DLD-1 cells, and together demonstrates that NFκB2 p100 processing is not dependent on a stimulus to activate LTβR. While NFκB2 activating receptors still must depend on a stimulus to initiate TRAF3 degradation, we show here, for the first time, that NFκB2 p100 processing is directly dependent on this degradation, and not on the stimulus per se. TRAF2 has also been shown to be a negative regulator of NFκB2 p100 processing (Grech, Amesbury et al. 2004), and unstimulated lymph node B cells from TRAF2-deleted mice have constitutively high levels of p52. Nevertheless, the levels of NFκB2 p100 processing into p52 in wild-type B cells in response to αCD40 and BAFF in the same report still correlate inversely to the levels of TRAF3 present (Grech, Amesbury et al. 2004). One possibility is that TRAF2 and TRAF3 together, in a non-exclusive process, control MK destabilization, and that the absence of either one of the components is sufficient to derepress NIK. However, in another report, Kim et. al (Kim, Nedospasov et al. 2005) fail to detect this basal NFκB2 activation in TRAF2 null cells, and our results are more similar to their finding.

Prior to the association with ligands, TNF receptors have been postulated to assemble into preformed complex through the pre-ligand assembly domain (PLAD) in the receptor (Chan, Chun et al. 2000). It was thus possible that LTβR also pre-associated into a complex, and that TRAF3 might play an important inhibitory role in signal transduction from this pre-associated complex. This would explain the ligand-independent activation of NFκB2 in the absence of TRAF3. This possibility was tested above by knocking down LTβR together with TRAF3, but found that NFκB2 activation was not affected by the loss of the receptor (FIG. 6D). These results, together with the observation that immunoprecipitates of pre-signaling LTβR-complexes did not contain appreciable amounts of the adapter proteins TRAF2 and TRAF3 (FIG. 3B ‘*’ lanes), strongly suggest that LTβR pre-assembly is not important with respect to signaling via the alternative arm.

The above-mentioned results from TRAF3 knockdown in DLD-1 cells also led us to some surprising and novel finding regarding the autoregulation of NFκB2. TRAF3 knockdown derepressed MK and allowed the stimulus-independent activation of NFκB2, and in addition, we detected significant increases in NFκB2 p100 and RelB transcripts. NFκB2 is a known transcriptional target of signaling via classical NFκB1 (Dejardin, Droin et al. 2002), however, we observed this increase in the absence of any detectable NFκB1 activation. Thus far, there is limited and conflicting evidence of NFκB2 p100 autoregulation, with a report that favors positive feedback (Liptay, Schmid et al. 1994) and another that favors negative feedback (Lombardi, Clana et al. 1995) of this transcription. However, both of these reports are based on measurements of transfected reporter activities, in contrast to the detection of endogenous transcripts in our study. In addition, our results show the mutual, co-dependent effect on RelB and p100 components of NFκB2, strengthening the proposed mechanism of positive autoregulation of this pathway. This positive autoregulation, when combined with a decrease in TRAF3 levels, provides a tempting explanation for the sustained activation of NFκB2 that is observed during signaling via LTβR and related receptors. Accordingly, how this sustained NFκB2 signal, facilitated by an autoregulatory positive-feedback loop, is eventually resolved, and whether this resolution requires yet another signal, an event that might resemble TNα-like resynthesis of TRAF3, are questions that still need to be answered.

### Summary of Examples 1.1-1.7

Examples 1.1-1.7 report that LTβR can selectively activate NFκB2 without activating NFκB1 in DLD-1 adenocarcinoma cells. An abundance of TRAF3 in LTβR-associated signaling complexes inhibits NFκB1 activation without affecting TNFα-dependent activation. Excess cytoplasmic TRAF3 results in its increased recruitment to LTβR-complexes in competition with TRAF2, preventing IκBα and RelA phosphorylation. siRNA mediated knockdown of TRAF3 enhances TRAF2 and 1IKKα recruitment to the LTβR-complexes upon receptor engagement, and restores NFκB1 signaling and NFκB1-dependent gene activation. TRAF3 knockdown also results in signal-independent processing of NFκB2 p100 into p52, by increasing the stabilization of NIK Furthermore, the loss of TRAF3 leads to an increase in p100 and RelB, revealing an auto-activation loop for the synthesis of alternative NFκB-arm components. These findings show that TRAF3 is directly linked to the activation of both classical and alternative NFκB pathways: as a molecular switch that selectively uncouples LTβR from signal-dependent NFκB1 activation, and as an inhibitor of signal-independent NFκB2 processing. Modulation of TRAF3 levels during receptor activation thus allows LTβR-specific control of diverse NFκB1- and NFκB2-dependent gene activation programs.

### Example 2

### Identification of Predictive Markers for Tumor Cells Susceptible to LTBR Treatment

p53 (mutant) expression was examined on tumor cells to determine whether there was a correlation between responsiveness to treatment with an LTβR activating agent and expression of p53.

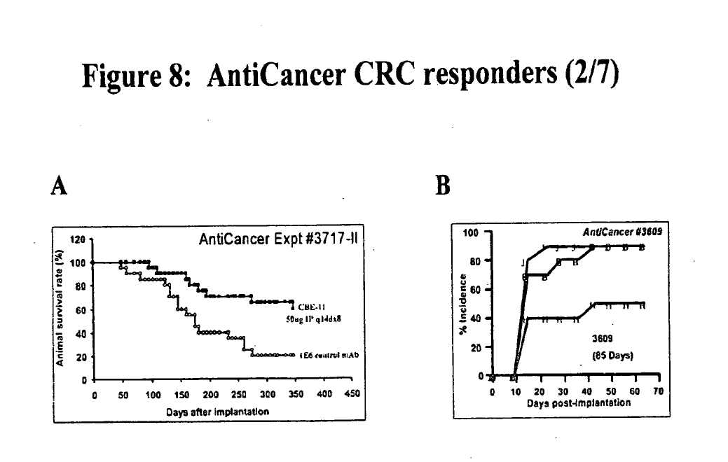

Human colorectal cancer cells (CRCs) were xenografted onto mice and used to determine whether CBE11, an LTβR activating agent, could inhibit tumor growth and increase survival in the experimental animals. p53 staining of AntiCancer orthotopic xenografts (n=7) showed a perfect correlation with CBE11 sensitivity. As shown in FIG. 8, CBE11 treated mice had improved survival over control mice. Staining of xenograft sections from orthotopic CRC models run by AntiCancer showed that CBE11 responders (2/7) were highly positive for p53.

In addition, a survey of human colorectal cancer tissues (60) showed that 30% are highly positive. A colorectal tumor array was surveyed, and revealed that 30% of human tumors are highly p53 positive. In addition, 30% of colon tumors showed heterogeneous p53 staining, while 40% of colon tumors were p53 negative.

In conclusion, tumor cell lines sensitive to CBE11 showed high expression of p53.

### EQUIVALENTS

The present invention provides among other things combination therapeutics involving LT-β-R antibodies. While specific embodiments of the subject invention have been discussed, the above specification is illustrative and not restrictive. Many variations of the invention will become apparent to those skilled in the art upon review of this specification. The full scope of the invention should be determined by reference to the claims, along with their full scope of equivalents, and the specification, along with such variations.

All publications and patents mentioned herein, including those items listed below, are hereby incorporated by reference in their entirety as if each individual publication or patent was specifically and individually indicated to be incorporated by reference. In case of conflict, the present application, including any definitions herein, will control.

1-30. (canceled)

31. A method for predicting the sensitivity or resistance of a tumor to treatment with an agent that specifically binds to and activates LT-β-R, comprising comparing an amount of TRAF3 present in the tumor prior to administration of the treatment with a control amount of TRAF3 and evaluating the amount of TRAF3 present in the tumor relative to the known standard amount, to thereby predict the sensitivity or resistance of a tumor to treatment with the agent, wherein the tumor is a carcinoma.

32. The method of claim 31, wherein the control amount is an amount of TRAF3 present in a tumor with known sensitivity to treatment with an LT-β-R activating agent, or a known standard amount of TRAF3 present in a tumor with known resistance to treatment with an LT-β-R activating agent.

33. The method of claim 32, wherein the tumor is predicted to be sensitive to the LT-β-R activating agent if the amount of TRAF3 present in the tumor is equal to or less than the known standard amount of TRAF3 present in a tumor with known sensitivity to treatment with the LT-β-R activating agent.

34. The method of claim 32, wherein the tumor is predicted to be resistant to the LT-β-R activating agent if the amount of TRAF3 present in the tumor is equal to or greater than the known standard amount of TRAF3 present in a tumor with known resistance to treatment with the LT-β-R activating agent.

35. The method of claim 31, wherein the method further comprises measuring the amount of TRAF2 present in a tumor.

36. The method of claim 35, wherein the amount of TRAF3 and the amount of TRAF2 are evaluated in a ratio.

37. The method of claim 36, wherein the ratio of TRAF3/TRAF2 is evaluated.

38. The method of claim 36, wherein the ratio of TRAF2/TRAF3 is evaluated.

39. The method of claim 37, wherein the tumor is predicted to be sensitive to the LT-β-R activating agent if the TRAF3/TRAF2 ratio present in the tumor is equal or less than the known standard TRAF3/TRAF2 ratio present in a tumor with known sensitivity to treatment with the LT-β-R activating agent.

40. The method of claim 37, wherein the tumor is predicted to be resistant to the LT-β-R activating agent if the TRAF3/TRAF2 ratio present in the tumor is equal to or greater than the known standard TRAF3/TRAF2 ratio present a tumor with known resistance to treatment with the LT-β-R activating agent.

41. The method of claim 31, wherein the amount of TRAF3 is measured by determining the level of expression of TRAF3 at the nucleic acid or protein level.

42. The method of claim 41, wherein the level of expression of TRAF3 is determined using an experimental technique selected from the group comprising Western blot, Northern blot, Southern blot, immunohistochemistry, ELISA, immunoprecipitation, immunofluorescence, electrophoresis, immunoelectrophoresis, high performance liquid chromatography, thin layer chromatography, radioimmunoassay, flow cytometry, immunocytochemistry, mass spectrometrometric analysis, polymerase chain reaction, probe arrays, nucleic acid hybridization techniques, nucleic acid reverse transcription methods and nucleic acid amplification methods.

43. The method of claim 41 or 42, wherein the expression level of TRAF3 is determined by using a primer or probe specific for TRAF3.

44. The method of claim 35, wherein TRAF2 is measured by determining the level of expression of TRAF2 at the nucleic acid or protein level.

45. The method of claim 44, wherein the level of expression of TRAF2 is determined using an experimental technique selected from the group comprising Western blot, Northern blot, Southern blot, immunohistochemistry, ELISA, immunoprecipitation, immunofluorescence, electrophoresis, immunoelectrophoresis, high performance liquid chromatography, thin layer chromatography, radioimmunoassay, flow cytometry, immunocytochemistry, mass spectrometrometric analysis, polymerase chain reaction, probe arrays, nucleic acid hybridization techniques, nucleic acid reverse transcription methods and nucleic acid amplification methods.

46. The method of claim 44 or 45, wherein the expression level of TRAF2 is determined by using a primer or probe specific for TRAF2,

47. The use of the LT-β-R activating agent of claim 31 or 35 and an agent that inhibits TRAF3 activity, in the preparation of a medicament for treating a subject having a tumor, wherein the tumor is a carcinoma, and wherein agent that inhibits TRAF3 is selected from the group consisting of an antibody, an siRNA molecule, and an antisense nucleic acid molecule.

48. The use of claim 47, wherein the siRNA molecules that inhibit TRAF3 activity are selected from the group consisting of:
(a) Sense strand siRNA: AGAAGUGAUGCCACUUGGUUU/
Antisense strand siRNA: ACCAAGUGGCAUCACUUCUUU;
(b) Sense strand siRNA: GUACAAGUGUGAGAAGUGCUU/
Antisense strand siRNA: GCACUUCUCACACUUGUACUU;
(c) Sense strand siRNA: GGUCUUGAGGAAAGACCUGUU/
Antisense strand siRNA: CAGGUCUUUCCUCAAGACCUU;
(d) Sense strand siRNA: UGGAGUGCUCAUCUGGAAGUU/
Antisense strand siRNA: CUUCCAGAUGAGCACUCCAUU;
and
(e) Sense strand siRNA: CUCCUCUGGGGGAUUUGAAUU/
Antisense strand siRNA: UUCAAAUCCCCCAGAGGAGUU

49. Use of the medicament of claim 47, wherein the medicament further comprises a chemotherapeutic agent, and wherein said chemotherapeutic agent is preferably selected from the group consisting of gemcitabine, adriamycin, Camptosar, carboplatin, cisplatin, and Taxol.

50. The method of claim 31, wherein the agent that binds to and activates LT-βR is selected from the group consisting of:
(a) an anti-LT-β-R binding molecule;
(b) an anti-LT-β-R antibody, or an antigen-binding fragment thereof;
(c) an anti-LT-β-R antibody that is a humanized antibody, or an antigen-binding fragment thereof;
(d) an anti-LT-β-R antibody that is a humanized antibody, or an antigen-binding fragment thereof, wherein said antibody or fragment comprises a variable region comprising complementary determining regions (CDRs) corresponding to CDRs from the mouse CBE11 antibody;
(e) an anti-LT-β-R antibody that is humanized CBE11;
(f) a multivalent anti-LT-β-R antibody;
(g) a multivalent anti-LT-β-R antibody that comprises at least one CDR derived from the CBE11 antibody; and
(h) anti-LT-β-R antibody or an antigen-binding fragment according to any one of (b) to (g) which is conjugated to a chemotherapeutic agent or an immunotoxin, wherein said chemotherapeutic agent is preferably selected from the group consisting of gemcitabine, adriamycin, Camptosar, carboplatin, cisplatin, and Taxol.

51. The method or agent of claim 31 or 35, wherein the tumor is a carcinoma.

52. The method or agent of claim 31 or 35, wherein the tumor is a colon tumor or a cervical tumor.

53. A kit for performing the method of claim 31 or 35, the kit comprising
(a) a detectable agent that specifically recognizes TRAF3, wherein the agent is used for determining the amount of TRAF3 present in a tumor;
(b) a detectable agent that specifically recognizes TRAF2, wherein the agent is used for determining the amount of TRAF2 present in the tumor of (a);
(c) instructions, which provide for evaluating the amount of TRAF3 in a tumor and the amount of TRAF2 in the tumor in a ratio; and
(d) instructions, which provide for comparing the TRAF3/TRAF2 ratio present in the tumor with the known standard TRAF3/TRAF2 ratio present in a tumor with known sensitivity to treatment with the LT-β-R activating agent; and/or
(e) instructions, which provide for comparing the TRAF3/TRAF2 ratio present in the tumor with the known standard TRAF3/TRAF2 ratio present in a tumor with known resistance to treatment with the LT-β-R activating agent.

54. A kit for performing the method of claim 31 or 35, the kit comprising
(a) a detectable agent that specifically recognizes TRAF3, wherein the agent is used for determining the amount of TRAF3 present a tumor; and
(b) instructions, which provide for comparing the amount of TRAF3 present in the tumor with the known standard amount of TRAF3 present in a tumor with known sensitivity to treatment with the LT-β-R activating agent; and/or
(c) instructions, which provide for comparing the amount of TRAF3 present in the tumor with the known standard amount of TRAF3 present in a tumor with known resistance to treatment with the LT-β-R activating agent.
